SEQUENCE LISTING (117 sequences)

SEQ ID NO: 1 - protein, 15 aa, Artificial Sequence
  PNNL yeast scFv library linker sequence
GGGGSGGGGSGGGGS

SEQ ID NO: 2 - DNA, 45 bp, Artificial Sequence
  Linker DNA Sequence
ggtggcggtggcagcggcggtggtggttccggaggcggcggttct

SEQ ID NO: 3 - protein, 5 aa, Artificial Sequence
  Furin Short Recognition Sequence
RKKRS

SEQ ID NO: 4 - DNA, 15 bp, Artificial Sequence
  Furin Short Recognition Sequence DNA
agaaagaagagatct

SEQ ID NO: 5 - protein, 10 aa, Artificial Sequence
  Furin Long Recognition Sequence
NSRKKRSTSA

SEQ ID NO: 6 - DNA, 30 bp, Artificial Sequence
  Furin Long Recognition Sequence DNA
aactcgagaaagaagagatctacctccgct

SEQ ID NO: 7 - protein, 7 aa, Artificial Sequence
  TEV Protease Recognition Sequence
ENLYFQG

SEQ ID NO: 8 - DNA, 21 bp, Artificial Sequence
  TEV Protease Recognition Sequence DNA
gaaaacctatacttccaaggt

SEQ ID NO: 9 - protein, 8 aa, Artificial Sequence
  HRV-3C Protease Recognition Sequence
LEVLFQGP

SEQ ID NO: 10 - DNA, 24 bp, Artificial Sequence
  HRV-3C Protease Recognition Sequence DNA
ttggaagttttgttccaaggtcca

SEQ ID NO: 11 - protein, 4 aa, Artificial Sequence
  Caspase 1 Protease Recognition Sequence
YVAD

SEQ ID NO: 12 - DNA, 12 bp, Artificial Sequence
  Caspase 1 Protease Recognition Sequence DNA
tacgttgctgac

SEQ ID NO: 13 - protein, 4 aa, Artificial Sequence
  Caspase 3 Protease Recognition Sequence
DEVD

SEQ ID NO: 14 - DNA, 12 bp, Artificial Sequence
  Caspase 3 Protease Recognition Sequence DNA
gacgaagttgac

SEQ ID NO: 15 - protein, 6 aa, Artificial Sequence
  MMP25 Recognition Sequence
VMRLVV

SEQ ID NO: 16 - DNA, 18 bp, Artificial Sequence
  MMP25 Recognition Sequence DNA
gtcatgagattggtcgtc

SEQ ID NO: 17 - protein, 9 aa, Artificial Sequence
  PKA Kemptide Recognition Sequence
LLRRASLGP

SEQ ID NO: 18 - DNA, 27 bp, Artificial Sequence
  PKA Kemptide DNA
ttgttgagaagagctagtttgggtcca

SEQ ID NO: 19 - protein, 11 aa, Artificial Sequence
  H3-K56 histone acetyltransferase recognition sequence
IRRFQKSTDLL

SEQ ID NO: 20 - DNA, 33 bp, Artificial Sequence
  H3-K56 HAT Recognition Sequence DNA
atcagaagattccaaaagtccaccgacttgttg

SEQ ID NO: 21 - protein, 252 aa, Artificial Sequence
  scFv sequence
QVQLVESEAEVKKPGSSVKVSCKASGGTFSSYAISWVRQAPGQGLEWMGGIIPIFGTANYAQKFQGRVTITADESTSTAYMELSSLRSEDTAVYYCVLLDTTMVTGYYFDYWGQGTLVTVSSGILGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 22 - DNA, 1221 bp, Artificial Sequence
  scFv sequence
cattttcaattaagatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctggtggaatctgaggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgtcttgttggatacaactatggttacgggatactactttgactactggggccagggaaccctggtcaccgtctcctcaggaattctaggatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 23 - protein, 264 aa, Artificial Sequence
  scFv sequence
QVQLVESEGGLVQPGGSLRLSCAASGFTFSSYEMNWVRQAPGKGLEWVSRIDGDGSSTNYADSVKGRFTISRDNAKSTLYLQMNSLRAEDTAVYYCTRARYFGSVSPYGMDVWGQGTTVTVSSGILGSGGGGSGGGGSGGGGSDIRVTQSPSSVSASVGDRVTISCRASQGIATWLGWYQQKPGKPPQLLIYSASTLQTGVPSRFSGSGSGTDFTLTISSLQPEDVATYYCQEGSTFPLTFGGGTKVDIKSGILEQKLISEEDL

SEQ ID NO: 24 - DNA, 881 bp, Artificial Sequence
  scFv sequence
ctctgcaggctagtggtggtggtggttctggtggtggtggttctgctagccaggtgcagctggtggagtctgagggaggcttggtacagcctggagggtccctgagactctcctgtgcagcctctggattcaccttcagtagttatgaaatgaactgggtccgccaggctccaggtaaggggctggagtgggtctcacgtattgatggtgatgggagcagcacaaactacgcggactccgtgaagggccgattcaccatctccagagacaacgccaagagcacgctgtatctgcaaatgaatagtctgagagccgaggacacggctgtgtattactgtacaagggccagatactttggttcggtgagcccctacggtatggacgtctggggccaagggaccacggtcaccgtctcctcaggaattctaggatccggtggcggtggcagcggcggtggtggttccggaggcggcggttctgacatccgggtgacccagtctccttcttccgtgtctgcatctgtgggtgacagagtcaccatcagttgtcgggcgagtcaggggattgccacctggttaggctggtatcagcagaagccagggaaaccccctcagctccttatctattctgcatccactttgcaaactggggtcccatcaaggttcagcggcagtggatctgggacagatttcactcttaccatcagcagcctgcagccggaggatgttgcaacttactattgtcaagagggtagcactttccctctcactttcggcggagggaccaaagtggatatcaaatccggaattctagaacaaaagcttatttctgaagaagacttgtaatagctcggcggccgcatcgagatctgataacaacag

SEQ ID NO: 25 - protein, 268 aa, Artificial Sequence
  scFv sequence
QVQLQQWDAGLVKPSQTLSLTCAISGDSVSSNSAAWNWIRQSPSRGLEWPGRTYYRSKWQNNYALSVQGRITINPDTSNNQFSLQLDSMTPEDTGVYYCTRGGGSLDYWGQGTLVTVSSGSASAPTGILGSGGGGSGGGGSGGGGSSYELTQPPSVSVSPGQTATITCSGDEMGDKYAYWYQQKPGQAPVLVIYKDSERPSGIPERFSGSSSGTTVTLTISGVQAEDEADYYCQSADSSGTSVVFGGGTKVTVLSGILEQKLISEEDL

SEQ ID NO: 26 - DNA, 908 bp, Artificial Sequence
  scFv sequence
ctctgcaggctagtggtggtggtggttctggtggtggtggttctggtggtggtggttctgctagccaggtgcagctacagcagtgggacgcaggactggtgaagccctcgcagaccctctcactcacctgtgccatctccggggacagtgtctctagcaacagtgctgcttggaactggatcaggcagtccccatcgagaggtcttgagtggccgggaaggacatactacaggtccaagtggcaaaacaattatgcactctctgtgcaaggtcgaataaccatcaacccagacacatccaacaaccaattctccctgcagctggactctatgactcccgaggacacgggtgtatattactgtacaaggggcggcgggtccttagactactggggccagggaaccctggtcaccgtctcctcagggagtgcatccgccccaaccggaattctaggatccggtggcggtggcagcggcggtggtggttccggaggcggcggttcttcctatgagctgacacagccaccctcagtgtccgtgtccccaggacagacagccaccatcacctgctctggagatgaaatgggggataaatatgcttattggtaccagcagaagccaggccaggcccctgtgctggtgatatataaagacagtgagaggccctcagggatccctgagcgattctctggctccagctcagggacaacagtcaccttgaccatcagtggagtccaggcagaagacgaggctgactattactgtcaatcagcagacagcagtggtacttctgtggtattcggcggagggaccaaggtcaccgtcctatccggaattctagaacaaaagcttatttctgaagaagacttgtaatagctcggcggccgcatcgagatctgataacaacag

SEQ ID NO: 27 - protein, 279 aa, Artificial Sequence
  scFv sequence
QVQLQQSGPGRVKPSQTLSLTCDISGDSVSSNSVAWNWIRQSPSRGLEWLGRTYYRSKWINEYGPFVRSRITINPDTSKNQFSLQLNSVTPEDTAVYYCATMANSGYDRSSGHNYGMDVWGQGTTVTVSSGSASAPTGILGSGGGGSGGGGSGGGGSSYELTQPPSVSVSPGQTARITCSGDALPKQYTYWYQQKAGQAPVLVIYKDTERPSGIPERFSGTSSGTTVTLTISGVQAEDEADYYCQSADSSGSYVFFGGGTKVTVLSGILEQKLISEEDL

SEQ ID NO: 28 - DNA, 935 bp, Artificial Sequence
  scFv sequence
cgctctgcaggctagtggtggtggtggttctggtggtggtggttctggtggtggtggttctgctagccaggtacagctgcagcagtcaggtccaggacgggtgaagccctcgcagaccctctcactcacctgtgacatctccggggacagtgtctctagcaacagtgttgcttggaactggatcaggcagtccccatcgagaggccttgagtggctgggaaggacatactacaggtccaagtggattaatgaatatggaccatttgtaagaagtcgaataaccatcaacccagacacatccaagaatcagttctccctgcagttgaactctgtgactcccgaggacacggctgtctattactgtgcaacaatggcgaatagtggctacgatcggtcctctggtcacaactacggaatggacgtctggggccaagggaccacggtcaccgtctcctcagggagtgcatccgccccaaccggaattctaggatccggtggcggtggcagcggcggtggtggttccggaggcggcggttcttcctatgagttgactcagccaccctcggtgtcagtgtccccaggacagacggccaggatcacctgctctggagatgcattgccaaagcaatatacttattggtaccagcagaaggcaggccaggcccctgtcttggtgatatataaagacactgagaggccctcagggatccctgagcgattctctggtaccagttcagggacaacagtcacattgaccatcagtggagtccaggcagaagacgaggctgactattactgtcaatcagcagacagcagtggttcctatgttttcttcggcggagggaccaaggtgaccgtcctatccggaattctagaacaaaagcttatttctgaagaagacttgtaatagctcggcggccgcatcgagatctgat

SEQ ID NO: 29 - protein, 123 aa, Artificial Sequence
  scFv sequence
QAVVTQEPSVTVSPGGTVILTCGSSTGAVTSGHYANWFQQKPGQAPRALIFETDKKYSWTPGRFSGSLLGAKAALTISDAQPEDEAEYYCLLSDVDGYLFGGGTQLTVLSGILEQKLISEEDL

SEQ ID NO: 30 - DNA, 651 bp, Artificial Sequence
  scFv sequence
ggctagtggtggtggtggttctggtggtggtggttctgctagcactggcagctttgactcctggggccagggaaccctggtcaccgtctcctcaggaattctaggatccggtggcggtggcagcggcggtggtggttccggaggcggcggttctcaggctgtggtgactcaggagccgtcagtgactgtgtccccaggagggacagtcattctcacttgtggctccagcactggagctgtcaccagtggtcattatgccaactggttccagcagaagcctggccaagcccccagggcacttatatttgaaaccgacaagaaatattcctggacccctggccgattctcaggctccctccttggggccaaggctgccctgaccatctcggatgcgcagcctgaagatgaggctgagtattactgtttgctctccgacgttgacggttatctgttcggaggaggcacccagctgaccgtcctctccggaattctagaacaaaagcttatttctgaagaagacttgtaatagctcggcggccgcatcgagatctgataacaacagtgtagatgtaacaaaatcgactttgttcccactgtacttttagctcgtacaaaatacaatatacttttcatttctccgtaaacaacatg

SEQ ID NO: 31 - protein, 124 aa, Artificial Sequence
  scFv sequence
QVQLQQSGPGLVRPSQTLSLTCAISGDSVPKNGASWNWIRLSPSRGLEWLGRTHYSSRWYHDYAFFVKSRITINVDTSETQVSLQLDSVTPDDTGVYYCARESQRRGWFDLWGQGTLVTVSQEF

SEQ ID NO: 32 - DNA, 853 bp, Artificial Sequence
  scFv sequence
ggtggtggtggttctggtggtggtggttctgctagccaggtacagctgcagcagtcaggtccaggactggtgaggccctcgcagaccctctcactcacctgtgccatctccggggacagtgtccctaagaacggtgcatcttggaactggatcaggctgtcaccatcgcgaggccttgagtggctgggaaggactcactacagttccaggtggtatcatgattatgcattctttgtgaagagtcgaataaccatcaacgtagatacatccgagacccaagtcagtctgcagctggactctgtgactcccgacgacacgggtgtttattactgtgcaagagaatctcaacgtaggggatggttcgacctctggggccagggaaccctggtcaccgtctcccaggaattctaggatccggtggcggtggcagcggcggtggtggttccggaggcggcggttctaattttatgctgactcagccccactctgtgtcggagtctccggggaggacggttaccttctcctgcacccgcagcagtggcggcattgccagcaactatgtgcagtggtaccaacagcgcccgggcagtacccccaccactgtgatctatggggatagccaaagaccctctggagtccctgatcggttctctggctccatcgacagctcctccaattctgcctccctcaccatctcagggctgaaggctgaggacgaggctgactactactgtcagtcctctgatggtagctcttgggtgttcggcggaggcacccagctgaccgtcctctccggaattctagaacaaaagcttatttctgaagaagacttgtaatagctcggcggcggcat

SEQ ID NO: 33 - protein, 154 aa, Artificial Sequence
  scFv sequence
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSGGGGSGGGGSGGGGLQEF

SEQ ID NO: 34 - DNA, 781 bp, Artificial Sequence
  scFv sequence
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatccggtggcggtggcagcggcggtggtggttccggaggcggcgggctgcaggaattctagaacaaaagcttatttctgaagaagacttgtaatagctcggcggccgcatcgagatctgataacaacagtgtagatgtaacaaaatcgactttgttcccactgtacttttagctcgtacaaaatacaatatacttttcatttctccgtaaacaacatg

SEQ ID NO: 35 - protein, 252 aa, Artificial Sequence
  scFv sequence
QVQLVESEAEVKKPGSSVKVSCKASGGTFSSYAISWVRQAPGQGLEWMGGTIPIFGTADYAQEFQGRVTITTDESTSTAYMELSGLRSEDTAVYYCVLLGTTMVTGHYFDYWGQGTLVTVSSGILGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDGLSGYVFGTGTKLTVL

SEQ ID NO: 36 - DNA, 845 bp, Artificial Sequence
  scFv sequence
ggttctggtggtggtggttctgctagccaggtgcagctggtggaatctgaggctgaggtgaagaagcctgggtcctcggtgaaggtctcctgcaaggcctctggaggcaccttcagcagctatgctatcagctgggtgcggcaggcccctggacaagggcttgagtggatgggagggaccatccctatctttggtacagcagactacgcacaggagttccagggcagagtcacgattaccacggacgaatccacgagcacagcctacatggagctgagcggcctgagatctgaggacacggccgtgtattactgtgttttgttgggtacaactatggttacgggacactactttgactactggggccagggaaccctggtcaccgtctcctcaggaattctaggatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacggtctgagtggttatgtcttcggaactgggaccaagcttaccgtcctgtccggatccgaacaaaagcttatttctgaagaggacttgtaatagctcggcggccgcatcga

SEQ ID NO: 37 - protein, 252 aa, Artificial Sequence
  scFv sequence
QVQLVESEAEVKKPGSSVKVSCKASGGTFSSYAISWVRQAPGQGLEWMGGTIPIFGTANYAQKFQGRVTITADESTSTAYMELSSLRSEDTAVYYCVLLGTTMVTGYYFDYWGQGTLVTVSSGILGSGGGGSGGGGSGGGGSNFTLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 38 - DNA, 864 bp, Artificial Sequence
  scFv sequence
gctctgcaggctagtggtggtggtggttctggtggtggtggttctggtggtggtggttctgctagccaggtgcagctggtggaatctgaggctgaggtgaagaagcctgggtcctcggtgaaggtctcctgcaaggcttctggaggcaccttcagcagctatgctatcagctgggtgcgacaggcccctggacaagggctcgagtggatgggagggaccatccctatctttggtacagcaaactacgcacagaagttccagggcagagtcacgattaccgcggacgagtccacgagcacagcctacatggagctgagcagcctgagatctgaggacacggccgtgtattactgtgtcttgttgggtacaactatggttacgggatactactttgactactggggccagggaaccctggtcaccgtctcctcaggaattctaggatccggtggcggtggcagcggcggtggtggttccggaggcggcggttctaattttacgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttg

SEQ ID NO: 39 - protein, 250 aa, Artificial Sequence
  scFv sequence
QVQLQQGGAGLLKPSETLSLTCGVYGGSFSGYYWSWIRQSPGKGLEWIGEINHSGSANYNPSVKSRVTISVDTSKNQFSLQLSSVTAADTAVYYCARDRAVLTGEGWYFDLWGRGTLVTVSSGILGSGGGGSGGGGSGGGGSSYELTQPPSVSVSPGQTASITCSGDKLGDKYTCWYQQKPGQSPVLVLYEDTKRPSGIPERFSGSNSGNTATLTISRVEAGDEADYYCQLWDSSSDHYVFGSGTKLTVL

SEQ ID NO: 40 - DNA, 835 bp, Artificial Sequence
  scFv sequence
ctggtggtggtggttctgctagccaggtgcagctacagcaggggggcgcaggactgttgaagccttcggagaccctgtccctcacgtgcggtgtctatggtgggtctttcagtggttactattggagctggattcgccagtccccaggaaaggggctggaatggattggggaaatcaatcatagtggaagcgccaactacaacccgtccgtcaagagtcgtgtcaccatatcagtagacacgtccaagaatcagttctccctgcagttgagctctgtgaccgctgcggacacggccgtgtactactgtgcgagagatagggcggtgttaacgggggagggctggtacttcgatctctggggccgtggtaccctggtcaccgtctcctcaggaattctaggatccggtggcggtggcagcggcggtggtggttccggaggcggcggttcttcctatgagctgacacagccaccctcagtgtccgtgtccccaggacagacagccagcatcacctgctctggagataaattgggggataaatatacttgttggtatcagcagaagccaggccagtcccctgtactggtcctctatgaagataccaagcggccctcagggatccctgagcgattctctggctccaactctgggaacacggccaccctgaccatcagcagggtcgaagccggggatgaggccgactattactgtcagctgtgggatagtagtagtgatcattatgtcttcggaagtgggaccaagctgaccgtcctatccggaattctagaacaaaagcttatttcagaagaagacttgtaatagctcggcggccgcat

SEQ ID NO: 41 - protein, 268 aa, Artificial Sequence
  H6-MG (VH) / HL1-TO1 (VL) - furin (short) biosensor (protein)
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSRKKRSGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 42 - DNA, 1033 bp, Artificial Sequence
  H6-MG (VH) / HL1-TO1 (VL) - furin (short) biosensor (DNA)
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatcgagaaagaagagatctggatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 43 - protein, 273 aa, Artificial Sequence
  H6-MG (VH) / HL1-TO1 (VL) - furin (long) biosensor (protein)
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSNSRKKRSTSAGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 44 - DNA, 1048 bp, Artificial Sequence
  H6-MG (VH) / HL1-TO1 (VL) - furin (long) biosensor (DNA)
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatcgaactcgagaaagaagagatctacctccgctggatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 45 - protein, 270 aa, Artificial Sequence
  H6-MG (VH) / HL1-TO1 (VL) - TEV protease biosensor (protein)
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSENLYFEGRSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 46 - DNA, 1039 bp, Artificial Sequence
  H6-MG (VH) / HL1-TO1 (VL) - TEV protease biosensor (DNA)
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatctgaaaacctatacttccaaggtcgatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 47 - protein, 271 aa, Artificial Sequence
  H6-MG (VH) / HL1-TO1 (VL) - HRV-3C protease biosensor (protein)
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSLEVLFQGPGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 48 - DNA, 1042 bp, Artificial Sequence
  H6-MG (VH) / HL1-TO1 (VL) - HRV-3C protease biosensor (DNA)
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatctttggaagttttgttccaaggtccaggatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 49 - protein, 267 aa, Artificial Sequence
  H6-MG (VH) / HL1-TO1 (VL) - Caspase 1 biosensor (protein)
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSYVADGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 50 - DNA, 1030 bp, Artificial Sequence
  H6-MG (VH) / HL1-TO1 (VL) - Caspase 1 biosensor (DNA)
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatcttacgttgctgacggatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 51 - protein, 267 aa, Artificial Sequence
  H6-MG (VH) / HL1-TO1 (VL) - Caspase 3 biosensor (protein)
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSDEVDRSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 52 - DNA, 1030 bp, Artificial Sequence
  H6-MG (VH) / HL1-TO1 (VL) - Caspase 3 biosensor (DNA)
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatctgacgaagttgaccgatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 53 - protein, 269 aa, Artificial Sequence
  H6-MG (VH) / HL1-TO1 (VL) - MMP25 biosensor (protein)
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSVMRLVVGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 54 - DNA, 1036 bp, Artificial Sequence
  H6-MG (VH) / HL1-TO1 (VL) - MMP25 biosensor (DNA)
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatcggtcatgagattggtcgtcggatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 55 - protein, 272 aa, Artificial Sequence
  H6-MG (VH) / HL1-TO1 (VL) - PKA biosensor (protein)
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSLLRRASLGPGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 56 - DNA, 1045 bp, Artificial Sequence
  H6-MG (VH) / HL1-TO1 (VL) - PKA biosensor (DNA)
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatcgttgttgagaagagctagtttgggtccaggatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 57 - protein, 274 aa, Artificial Sequence
  H6-MG (VH) / HL1-TO1 (VL) - H3-K56 HAT biosensor (protein)
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSIRRFQKSTDLLGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 58 - DNA, 1051 bp, Artificial Sequence
  H6-MG (VH) / HL1-TO1 (VL) - H3-K56 HAT biosensor (DNA)
actagcaaaggcagccccataaacacacagtatgtttttaaggacaatagctcgacgattgaaggtagatacccatacgacgttccagactacgctctgcaggctagtggtggtggtggttctggtggtggtggttctggtggtggtggttctgctagccaggtgcagctgcaggagtcgggcccaggactggtgaagccttcggagaccctgtccctcacctgcactgtctctggtgcctccatcagcagtagtcattactactggggctggatccgccagcccccagggaaggggcctgagtggattgggagtatgtattatagtgggagaacgtactacaacccggccctcaagagtcgagtcaccatatcaccagacaagtcgaagaaccagttcttcttgaagttgacctctgtaaccgccgcggacacggccgtgtattactgtgcgagggagggacccacacattactatgataatagtggtccaataccttcggatgagtatttccagcactggggccagggtaccctggtcactgtctcctcaggaattctaggatcgatcagaagattccaaaagtccaccgacttgttgggatccggtggcggtggcagcggcggtggtggttccggaggcggcggttctaattttatgctgactcagcccccctcagcgtctgggacccccgggcagagcgtcaccatctcttgttctggaagcggctcgaacatcggaaacaataaagtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctcaccgtcctatccggaattctagaacaaaagcttatttctgaagaagacttgtaatagctcggcggccgcatcgagatct

SEQ ID NO: 59 - DNA, 90 bp, Artificial Sequence
  Oligonucleotide primer
gatccggtggcggtggcagcggcggtggtggttccggcccaagcggccgcctggaagttctgttccaggggcccggcccgtcaggccgcg

SEQ ID NO: 60 - DNA, 90 bp, Artificial Sequence
  Oligonucleotide primer
gatccgcggcctgacgggccgggcccctggaacagaacttccaggcggccgcttgggccggaaccaccaccgccgctgccaccgccaccg

SEQ ID NO: 61 - DNA, 13 bp, Artificial Sequence
  SfiI recognition sequence
ggcccaagcggcc

SEQ ID NO: 62 - DNA, 13 bp, Artificial Sequence
  SfiI recognition sequence
ggcccgtcaggcc

SEQ ID NO: 63 - DNA, 13 bp, Artificial Sequence
  SfiI recognition sequence
ggcccaagcggcc

SEQ ID NO: 64 - DNA, 39 bp, Artificial Sequence
  Oligonucleotide primer
cggccgtgttcagctcgcgtattttaccgcgggcccgtc

SEQ ID NO: 65 - DNA, 39 bp, Artificial Sequence
  Oligonucleotide primer
gggcccgcggtaaaatacgcgagctgaacacggccgctt

SEQ ID NO: 66 - DNA, 66 bp, Artificial Sequence
  Oligonucleotide primer
cggccgtaccagaattgatgaggccaaccaacgtgcaacaaagatgctgggaagtggtggcccgtc

SEQ ID NO: 67 - DNA, 66 bp, Artificial Sequence
  Oligonucleotide primer
gggccaccacttcccagcatctttgttgcacgttggttggcctcatcaattctggtacggccgctt

SEQ ID NO: 68 - protein, 18 aa, Artificial Sequence
  Synaptosome-associated protein of 25 kDa (SNAP-25) target sequence
TRIDEANQRATKMLGSGG

SEQ ID NO: 69 - DNA, 45 bp, Artificial Sequence
  Oligonucleotide primer
tatataggcccaagcggccgtatggatgaaaacctagagcaggtg

SEQ ID NO: 70 - DNA, 40 bp, Artificial Sequence
  Oligonucleotide primer
tatataggcctgacgggcccccacttcccagcatctttgt

SEQ ID NO: 71 - protein, 61 aa, Artificial Sequence
  Synaptosome-associated protein of 25 kDa (SNAP-25) target protein
MDENLEQVSGIIGNLRHMALDMGNEIDTQNRQIDRIMEKADSNKTRIDEANQRATKMLGSG

SEQ ID NO: 72 - protein, 206 aa, Artificial Sequence
  Synaptosome-associated protein of 25 kDa (SNAP-25) target sequence
MAEDADMRNELEEMQRRADQLADESLESTRRMLQLVEESKDAGIRTLVMLDEQGEQLDRVEEGMNHINQDMKEAEKNLKDLGKCCGLFICPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 73 - DNA, 39 bp, Artificial Sequence
  Oligonucleotide primer
tatataggcccaagcggccgtatggccgaagacgcagac

SEQ ID NO: 74 - DNA, 13 bp, Artificial Sequence
  SfiI restriction enzyme recognition sequence
ggccnnnnnggcc

SEQ ID NO: 75 - DNA, 138 bp, Artificial Sequence
  Linker Extension Sequence
tccggtggcggtggcagcggcggtggtggttccggcccaagcggccgcctggaagttctgttccaggggcccggcccgtcaggccgcggatccggtggcggtggcagcggcggtggtggttccggaggcggcggttct

SEQ ID NO: 76 - protein, 47 aa, Artificial Sequence
  Linker Extension Sequence
GSGGGGSGGGGSGPSGRLEVLFQGPGPSGRGSGGGGSGGGGSGGGGS

SEQ ID NO: 77 - protein, 4 aa, Artificial Sequence
  Caspase 3 Protease Recognition Sequence
DEVD

SEQ ID NO: 78 - protein, 4 aa, Artificial Sequence
  Caspase 1 Protease Recognition Sequence
YAVD

SEQ ID NO: 79 - protein, 6 aa, Artificial Sequence
  MMP14 Protease Recognition Sequence
ARGIKL

SEQ ID NO: 80 - protein, 8 aa, Artificial Sequence
  MMP2 Protease Recognition Sequence
LQLALYTA

SEQ ID NO: 81 - protein, 8 aa, Artificial Sequence
  MMP2 Protease Recognition Sequence
VQLAYFTA

SEQ ID NO: 82 - protein, 6 aa, Artificial Sequence
  MMP2 Protease Recognition Sequence
ATLAAL

SEQ ID NO: 83 - protein, 6 aa, Artificial Sequence
  MMP2 Protease Recognition Sequence
FYFSNL

SEQ ID NO: 84 - protein, 6 aa, Artificial Sequence
  MMP2 Protease Recognition Sequence
GHPSPF

SEQ ID NO: 85 - protein, 6 aa, Artificial Sequence
  MMP2 Protease Recognition Sequence
GYFANL

SEQ ID NO: 86 - protein, 6 aa, Artificial Sequence
  MMP2 Protease Recognition Sequence
PKLAAI

SEQ ID NO: 87 - protein, 6 aa, Artificial Sequence
  MMP 2 & 9 Protease Recognition Sequence
RPSPFW

SEQ ID NO: 88 - protein, 22 aa, Artificial Sequence
  Thrombin Protease Recognition Sequence
TNATLDPRSFLLRNPNDKYEPF

SEQ ID NO: 89 - protein, 11 aa, Artificial Sequence
  Thrombin Protease Recognition Sequence
LDPRSFLLRNP

SEQ ID NO: 90 - protein, 7 aa, Artificial Sequence
  TEV Protease Recognition Sequence
ENLYFQG

SEQ ID NO: 91 - protein, 10 aa, Artificial Sequence
  HIV1 protease recognition sequence
VSQNYPIVQN

SEQ ID NO: 92 - protein, 10 aa, Artificial Sequence
  HIV1 Protease Recognition Sequence
KARVLAEAMS

SEQ ID NO: 93 - protein, 10 aa, Artificial Sequence
  HIV1 Protease Recognition Sequence
SATIMMQRGN

SEQ ID NO: 94 - protein, 10 aa, Artificial Sequence
  HIV1 Protease Recognition Sequence
ERQANFLGKI

SEQ ID NO: 95 - protein, 10 aa, Artificial Sequence
  HIV1 Protease Recognition Sequence
RPGNFLQSRP

SEQ ID NO: 96 - protein, 10 aa, Artificial Sequence
  HIV1 Protease Recognition Sequence
ERQANFLRED

SEQ ID NO: 97 - protein, 10 aa, Artificial Sequence
  HIV1 Protease Recognition Sequence
EDLAFLQGKA

SEQ ID NO: 98 - protein, 10 aa, Artificial Sequence
  HIV1 Protease Recognition Sequence
VSFNFPQVTL

SEQ ID NO: 99 - protein, 10 aa, Artificial Sequence
  HIV1 Protease Recognition Sequence
CTLNFPISPI

SEQ ID NO: 100 - protein, 10 aa, Artificial Sequence
  HIV1 Protease Recognition Sequence
GAETFYVDGA

SEQ ID NO: 101 - protein, 10 aa, Artificial Sequence
  HIV1 Protease Recognition Sequence
IRKVLFLDGI

SEQ ID NO: 102 - protein, 10 aa, Artificial Sequence
  HIV1 Protease Recognition Sequence
AACAWLEAQE

SEQ ID NO: 103 - protein, 10 aa, Artificial Sequence
  HIV1 Protease Recognition Sequence
ERQVNFLGKI

SEQ ID NO: 104 - protein, 10 aa, Artificial Sequence
  HIV1 Protease Recognition Sequence
KARVIAEAMS

SEQ ID NO: 105 - protein, 10 aa, Artificial Sequence
  HIV1 Protease Recognition Sequence
KARVFAEAMS

SEQ ID NO: 106 - protein, 10 aa, Artificial Sequence
  HIV1 Protease Recognition Sequence
KARVYAEAMS

SEQ ID NO: 107 - protein, 8 aa, Artificial Sequence
  HIV1 Protease Recognition Sequence
GDEMVSLK

SEQ ID NO: 108 - protein, 8 aa, Artificial Sequence
  HIV1 Protease Recognition Sequence
KHIYYITG

SEQ ID NO: 109 - protein, 20 aa, Artificial Sequence
  HIV1 Protease Recognition Sequence
GHSNQVSQNYPIVQNIQGQM

SEQ ID NO: 110 - protein, 30 aa, Artificial Sequence
  HIV1 Protease Recognition Sequence
AAADTGHSNQVSQNYPIVQNIQGQMVHQAI

SEQ ID NO: 111 - protein, 25 aa, Artificial Sequence
  HIV1 Protease Recognition Sequence
GVGGPGHKARVLAEAMSQVTNSATI

SEQ ID NO: 112 - protein, 10 aa, Artificial Sequence
  HCV-NS4A/4B Protease Recognition Sequence
DEMEECASHL

SEQ ID NO: 113 - protein, 22 aa, Artificial Sequence
  Trombin Protease Recognition Sequence
TNATLDPRSFLLRNPNDKYEPF

SEQ ID NO: 114 - protein, 9 aa, Artificial Sequence
  ADAM10 Protease Recognition Sequence
PRYEAYKMG

SEQ ID NO: 115 - protein, 9 aa, Artificial Sequence
  ADAM10 Protease Recognition Sequence
PRAEALKGG

SEQ ID NO: 116 - protein, 9 aa, Artificial Sequence
  TACE Protease Recognition Sequence
PRAAAVKSP

SEQ ID NO: 117 - protein, 9 aa, Artificial Sequence
  TNF Protease Recognition Sequence
PLAQAVRSS

(19) United States
(12) Patent Application Publication
(10) Pub. No.: US 2016/0033490 A1
(43) Pub. Date: Feb. 4, 2016

(54) Linked Peptide Fluorogenic Biosensors

(71) Applicant: CARNEGIE MELLON UNIVERSITY, Pittsburgh, PA (US)

(72) Inventor: Peter B. Berget, Philadelphia, PA (US)

(21) Appl. No.: 14/774,362

(22) PCT Filed: Mar. 15, 2013
(86) PCT No.: PCT/US13/31988

§ 371 Date: Sep. 10, 2015

(51) Int. Cl. G01N 33/542; C07K 16/00; G01N 33/573; C12N 15/90

(52) U.S. Cl. CPC G01N 33/542; C12N 15/907; C07K 16/00; G01N 33/573; C07K 2317/622; C07K 2317/56

(57) ABSTRACT

Biosensors, compositions comprising biosensors, methods of producing biosensors, and methods of using biosensors are disclosed. The biosensors comprise a fluorogen-activating peptide and a blocking peptide. The blocking peptide associates with the fluorogen-activating peptide thereby blocking an active domain of the fluorogen-activating peptide. The fluorogen-activating peptide and blocking peptide are covalently linked in certain embodiments through a peptide linker. The peptide linker may contain an amino acid sequence that is specifically recognized as a modification substrate by a cognate enzyme. The fluorogen-activating peptide and the blocking peptide at least partially disassociate when the linker is modified by a cognate enzyme, thereby allowing the fluorogen-activating peptide to bind a cognate fluorogen and modulate a fluorescence signal.

### PRIORITY

For purposes of the U.S. national stage of this application, this application is a continuation-in-part of co-pending U.S. patent application Ser. No. 12/745,882, which is a United States national stage application of and claims the benefit of International Application Number PCT/US2008/085415, filed Dec. 3, 2008, which claims the benefit of U.S. Provisional Patent Application Ser. No. 61/005,122, filed Dec. 3, 2007; the contents of each of which are incorporated by reference herein in their entirety.

### GOVERNMENT SUPPORT

The invention claimed herein was made in part with support from the United States Government under National Institutes of Health (NIH) Grant Number 1U54-RR022241. The United States Government has certain rights in this invention.

### TECHNICAL FIELD

The present disclosure is directed to biosensors, compositions comprising biosensors, methods of producing biosensors, and methods of using biosensors.

### SEQUENCE LISTING

This application includes a Sequence Listing submitted via EFS-Web in computer readable form contained in a 73,283 byte file entitled 080750CIPPCT_ST25.txt created on Mar. 12, 2013, which is hereby incorporated herein by reference in its entirety.

### BACKGROUND

The detection of target molecules and molecular components of larger structures is important in biological and biochemical sciences. The identification, analysis and monitoring of target biochemical or biological analytes, for example, is important for biomedical applications. Current diagnostics and assays employ a variety of methods to detect and analyze target molecules or molecular components (“analytes”) in various environments, both in vitro and in vivo. Certain detection and analysis methods employ fluorescence phenomena. For example, immunoassays often employ antibodies labeled with fluorescent dye molecules (e.g., fluorescein derivatives) to target and detect certain analytes that specifically interact with the antibody. In these methods, a fluorescence signal produced by the fluorescent dye molecule attached to the antibody correlates with antibody-analyte interaction.

In other methods, a fluorescence signal may be altered by interaction between an analyte and a biosensor. Biosensor methods are capable of detecting the activity of analytes such as enzymes. For example, biosensors based on fluorogenic protease substrates comprising casein conjugates of two boron-dipyrromethene (BODIPY) dyes have been shown to be capable of detecting protease activity. This type of biosensor is disclosed in Jones et al., Analytical Biochem., 251, 144-152 (1997). In another example, biosensors based on fluorescence resonance energy transfer (“FRET”) have been developed to detect kinase activity. A biosensor of this type includes a chimeric protein comprising a cyan fluorescent protein and a yellow fluorescent protein, which undergoes a conformational shift in response to phosphorylation. The conformational shift in the protein alters the orientation between the two fluorescent proteins and generates a FRET change. This type of biosensor is disclosed in Zhang et al., Proc. Natl. Acad. Sci. USA, 98, 14997-15002, 2001.

### SUMMARY

The present disclosure is directed in part to novel peptide constructs that find utility as biosensors in various applications.

Various embodiments disclosed herein are directed to linked peptide fluorogenic biosensors. The disclosed biosensors comprise a peptide construct comprising a fluorogen-activating peptide and a blocking peptide. In various embodiments, the fluorogen-activating peptide is linked to the blocking peptide through a peptide linker comprising an amino acid sequence that is specifically recognized as a modification substrate by a cognate enzyme. The blocking peptide associates with the fluorogen-activating peptide thereby blocking the active domain of the fluorogen-activating peptide when the peptide linker is in an unmodified state. The fluorogen-activating peptide and the blocking peptide at least partially disassociate when the linker is modified by a cognate enzyme, thereby allowing the fluorogen-activating peptide to bind a cognate fluorogen and modulate the fluorescence signal produced by the fluorogen.

In various embodiments, the invention provides a recombinant DNA molecule encoding a biosensor. A unique recombinant DNA molecule comprises a first DNA sequence encoding a fluorogen-activating peptide and a second DNA sequence encoding a blocking peptide, wherein one of the fluorogen-activating peptide and the blocking peptide comprises a variable heavy chain domain of an antibody and the other peptide comprises a variable light chain domain of a different antibody. In various preferred embodiments of the invention, the unique recombinant DNA molecule also comprises a convertible linker DNA sequence positioned between the first DNA sequence and the second DNA sequence, wherein the convertible linker DNA sequence comprises a first restriction enzyme cleavage site, a second restriction enzyme cleavage site, and a first target DNA sequence positioned between the first restriction enzyme cleavage site and the second restriction enzyme cleavage site such that the first target DNA sequence is excised upon digestion, in use, with at least one restriction enzyme that cleaves the first cleavage site and the second cleavage site. Preferably, the first cleavages site and the second cleavage site are cleavable by a first restriction enzyme. The unique recombinant DNA molecule has, upon digestion in use with the first restriction enzyme, two different, non-complementary overhang sequences. In those preferred embodiments where the DNA molecule is circular, the recombinant DNA molecule is linearized upon excision of the target sequence by digestion in use with a restriction enzyme that cleaves the first cleavage site and the second cleavage site. Such linearized recombinant DNA molecule will not recircularize absent ligation to a second target DNA sequence comprising non-complementary overhang sequences that are complimentary to the non-complementary overhang sequences of the recombinant DNA molecule. In various embodiments, the second target DNA sequence comprises a first overhang sequence and a second overhang sequence, wherein the first overhang sequence of the second target DNA sequence is complementary to one of the overhang sequences of the linearized recombinant DNA molecule and the second overhang sequence of the second target DNA sequence is complementary to the other overhang sequence of the linearized recombinant DNA molecule. For example, the second target DNA sequence has 5′ and 3′ ends, each complementary to one of the non-complementary overhang sequences of the recombinant DNA molecule in only one orientation. The structure of the unique recombinant DNA molecule, and in particular, the structure of the convertible linker allows for the quick removal and insertion of a variety of different target sequences to alter the DNA molecule and encode for a wide variety of biosensors having linkers that are cleavable by a variety of enzymes, such as those in the family of protease enzymes. The recombinant DNA molecule described herein serves as a platform for the development and construction of numerous specific biosensors. The platform includes the convertible linker segment that may be genetically manipulated to efficiently insert a variety of DNA or nucleotide sequences between the fluorogen activating peptide and the blocking peptide to create customized target DNA or nucleotide sequences coding for a variety of desired target amino acid sequences. As a result, the platform provides an efficient and cost effective means to enhance the family of the disclosed biosensors.

Various embodiments disclosed herein are also directed to methods for analyzing enzyme activity. The disclosed methods comprise contacting a medium comprising an analyte enzyme with a composition comprising a fluorogen and a biosensor construct as disclosed herein, and detecting a fluorescence signal produced by an interaction between the fluorogen-activating peptide of the biosensor construct and the fluorogen.

For example, methods are disclosed for analyzing enzyme activity by contacting a reaction medium suspected of containing at least one enzyme of interest with at least one composition comprising a biosensor and a cognate fluorogen, the biosensor being encoded by a unique recombinant DNA molecule, and detecting for a fluorescence signal produced by an interaction between at least one fluorogen-activating peptide and the cognate fluorogen thereof to determine the presence of at least one said enzyme. The enzyme in various embodiments is a protease. The fluorogen may be selected from the group consisting of thiazole orange, malachite green, dimethyl indole red, and derivatives thereof.

Various embodiments of the invention comprise a vector comprising a nucleic acid sequence of the unique recombinant DNA molecule and a host cell that carries such a vector.

Various embodiments of the invention comprise an isolated, purified biosensor encoded by the unique recombinant DNA molecule, and a host cell expressing such biosensor.

It should be understood that this disclosure is not limited to the embodiments disclosed in this Summary, and it is intended to cover modifications that are within the spirit and scope of the invention, as defined by the claims.

### BRIEF DESCRIPTION OF THE DRAWINGS

The characteristics and advantages of the present disclosure may be better understood by reference to the accompanying figures.

FIGS. 1A and 1B are diagrams illustrating the functionality of a biosensor construct according to various embodiments disclosed herein.

FIGS. 2A and 2B are diagrams illustrating the functionality of a biosensor construct according to various embodiments disclosed herein.

FIGS. 3A and 3B are diagrams illustrating the functionality of a biosensor construct according to various embodiments disclosed herein.

FIG. 4 is a diagram illustrating the structure of peptide constructs according to various embodiments disclosed herein.

FIG. 5 is a diagram illustrating a single-domain antibody comprising a variable heavy chain domain fragment, a blocked hybrid scFv comprising the variable heavy chain domain fragment, and a protease biosensor construct comprising the variable heavy chain domain fragment according to various embodiments disclosed herein.

FIG. 6 presents diagrams depicting the genetic structure of single chain antibodies in a pPNL6 plasmid as described herein; diagram (a) depicts a plasmid segment comprising DNA coding for a variable heavy chain antibody fragment and a variable light chain antibody fragment; diagram (b) depicts a plasmid segment comprising DNA coding for a variable heavy chain antibody fragment where the DNA coding for a variable light chain antibody fragment has been excised; diagram (c) depicts a plasmid segment comprising DNA coding for a variable light chain antibody fragment where the DNA coding for a variable heavy chain antibody fragment has been excised.

FIG. 7 presents qualitative plots of cytometric data for single chain antibodies and fragments thereof displayed on the surface of yeast (“FL” refers to full length single chain antibodies, “HO” refers to variable heavy single-domain antibody fragments, and “LO” refers to variable light single-domain antibody fragments).

FIG. 8 is a molecular schematic of a malachite green fluorogen derivative;

FIG. 9 is a graph presenting the results of a fluorogen titration analysis of a variable heavy single-domain antibody fragment (square-shaped data points) and a hybrid blocked single chain antibody (triangle-shaped data points).

FIG. 10 presents semi-quantitative plots of cytometric data for single chain antibodies displayed on the surface of yeast; the plots on the left side of the Figure correspond to c-myc surface expression; the plots on the right side of the Figure correspond to fluorogen activity; the plots on the top half of the Figure correspond to a variable heavy single-domain antibody fragment (as illustrated in the accompanying diagram); and the plots on the bottom half of the Figure correspond to a hybrid blocked single chain antibody comprising the variable heavy single-domain antibody fragment (as illustrated in the accompanying diagram).

FIG. 11A presents a diagram depicting the nucleotide and amino acid sequences in a peptide linker region in a single chain antibody construct. The portion of the amino acid sequence depicted in FIG. 11A beginning with the third “Gly” and ending with the sixth “Ser” represents SEQ ID NO:1. The portion of the nucleotide sequence beginning with the first “GGT” and ending with “TCT” represents SEQ ID NO:2. FIG. 11B presents a diagram detailing the cleavage of the DNA depicted in FIG. 11A by a restriction enzyme, and the ligation of an enzyme recognition sequence for HRV-3C protease into the peptide linker region; and FIG. 11C depicts the nucleotide and amino acid sequences in a peptide linker region having an enzyme recognition sequence spliced therein. The portion of the amino acid sequence in the peptide linker region, as depicted in FIG. 11C, beginning with the second “Leu” and ending with “Pro” represents SEQ ID NO:9. The portion of the nucleotide sequence beginning with the first “TTG” and ending with “CCA” represents SEQ ID NO:10.

FIG. 12 presents semi-quantitative plots of cytometric data for a hybrid blocked single chain antibody having a protease recognition sequence spliced therein as illustrated in the accompanying diagram.

FIG. 13 presents semi-quantitative plots of cytometric data for a hybrid blocked single chain antibody having a protease recognition sequence spliced therein and treated with cognate protease as illustrated in the accompanying diagram.

FIG. 14 is a graph presenting the results of a kinetic protease assay for an HRV-3C protease biosensor according to an embodiment disclosed herein.

FIG. 15 presents a diagram depicting the nucleotide sequence for a peptide linker region in a single chain antibody construct, and detailing the cleavage of the DNA by a restriction enzyme and the ligation of an enzyme recognition sequence for Caspase 3 protease into the peptide linker region. The portion of the forward oligonucleotide sequence beginning with the first “GAC” and ending with the second “GAC” represents the Caspase 3 protease recognition sequence, SEQ ID NO:14. The portion of the amino acid sequence beginning with the first “Asp” and ending with the second “Asp” represents the Caspase 3 protease recognition sequence, SEQ ID NO:13.

FIG. 16 is a graph presenting the results of a kinetic protease assay for a caspase 3 protease biosensor according to an embodiment disclosed herein.

FIG. 17 is photograph of an SDS gel of a single-domain antibody comprising a variable heavy chain domain fragment (H6-MG), a blocked hybrid scFv comprising the variable heavy chain domain fragment (BC1), and a caspase 3 protease biosensor construct comprising the variable heavy chain domain fragment according to an embodiment disclosed herein.

FIG. 18 presents microscopy images of HeLa cells injected with Cascade Blue dextran tracking solution containing isolated and purified biosensors according to an embodiment disclosed herein.

FIG. 19 is a diagram depicting a transmembrane fused HRV-3C protease biosensor according to an embodiment disclosed herein.

FIG. 20 presents microscopy images of NIH 3T3 cells transduced with a retroviral vector expressing the HRV-3C biosensor as illustrated in FIG. 18.

FIG. 21 presents a diagram depicting the nucleotide sequence for a peptide linker region in a single chain antibody construct, and detailing the cleavage of the DNA by a restriction enzyme and the ligation of an enzyme recognition sequence for Caspase 1 protease into the peptide linker region. The portion of the forward oligonucleotide sequence beginning with the nucleotides “TAC” and ending with “GAC” represents the Caspase 1 protease recognition sequence, SEQ ID NO:12. The portion of the amino acid sequence beginning with the amino acid “Tyr” and ending with “Asp” represents the Caspase 1 protease recognition sequence, SEQ ID NO: 11.

FIG. 22 presents a diagram depicting the nucleotide sequence for a peptide linker region in a single chain antibody construct, and detailing the cleavage of the DNA by a restriction enzyme and the ligation of an enzyme recognition sequence for TEV protease into the peptide linker region. The portion of the forward oligonucleotide sequence beginning with the nucleotides “GAA” and ending with “GGT” represents the TEV protease recognition sequence, SEQ ID NO:8. The portion of the amino acid sequence beginning with the amino acid “Glu” and ending with the second “Gly” represents the TEV protease recognition sequence, SEQ ID NO:7.

FIG. 23 presents a diagram depicting the nucleotide sequence for a peptide linker region in a single chain antibody construct, and detailing the cleavage of the DNA by a restriction enzyme and the ligation of an enzyme short recognition sequence for furin protease into the peptide linker region. The portion of the forward oligonucleotide sequence beginning with the first “AGA” and ending with “TCT” represents the Furin protease short recognition sequence, SEQ ID NO:4. The portion of the amino acid sequence beginning with the first “Arg” and ending with the second “Ser” represents the Furin protease short recognition sequence, SEQ ID NO:3.

FIG. 24 presents a diagram depicting the nucleotide sequence for a peptide linker region in a single chain antibody construct, and detailing the cleavage of the DNA by a restriction enzyme and the ligation of an enzyme long recognition sequence for furin protease into the peptide linker region. The portion of the forward oligonucleotide sequence beginning with “AAC” and ending with “GCT” represents the Furin protease long recognition sequence, SEQ ID NO:6. The portion of the amino acid sequence beginning with “Asn” and ending with “Ala” represents the Furin protease long recognition sequence, SEQ ID NO:5.

FIG. 25 presents SDS gels for purified and soluble furin protease biosensors treated with furin in the indicated molar ratios.

FIG. 26 presents graphs presenting the results of kinetic protease assays for purified and soluble furin protease biosensors according to an embodiment disclosed herein.

FIG. 27 presents a diagram depicting the nucleotide sequence for a peptide linker region in a single chain antibody construct, and detailing the cleavage of the DNA by a restriction enzyme and the ligation of an enzyme recognition sequence for MMP25 protease into the peptide linker region. The portion of the forward oligonucleotide sequence beginning with the first “GTC” and ending with the third “GTC” represents the MMP25 protease recognition sequence, SEQ ID NO:16. The portion of the amino acid sequence beginning with the first “Val” and ending with the third “Val” represents the MMP25 protease recognition sequence, SEQ ID NO:15.

FIG. 28 presents a diagram depicting the nucleotide sequence for a peptide linker region in a single chain antibody construct, and detailing the cleavage of the DNA by a restriction enzyme and the ligation of an enzyme recognition sequence for protein kinase A (PKA Kemptide phosphorylation) into the peptide linker region. The portion of the forward oligonucleotide sequence beginning with the first “TTG” and ending with “CCA” represents the PKA Kemptide phosphorylation recognition sequence, SEQ ID NO:18. The portion of the amino acid sequence beginning with the first “Leu” and ending with “Pro” represents the PKA Kemptide phosphorylation recognition sequence, SEQ ID NO:17.

FIG. 29 is a diagram illustrating a protein kinase A biosensor according to an embodiment disclosed herein.

FIG. 30 presents a diagram depicting the nucleotide sequence for a peptide linker region in a single chain antibody construct, and detailing the cleavage of the DNA by a restriction enzyme and the ligation of an enzyme recognition sequence for an acetyltransferase that acetylates certain lysine residues in histone H3 into the peptide linker region. The portion of the forward oligonucleotide sequence beginning with “ATC” and ending with the second “TTG” represents the H3-K56 acetylation recognition sequence, SEQ ID NO:20. The portion of the amino acid sequence beginning with “Ile” and ending with the second “Leu” represents the H3-K56 acetylation recognition sequence, SEQ ID NO:19.

FIG. 31 is diagram illustrating an H3 K56 acetyltransferase biosensor according to an embodiment disclosed herein.

FIG. 32A illustrates the nucleotide sequences annealed to generate a double stranded DNA molecule with BamH1 compatible overhang sequences.

FIG. 32B illustrates the translation of the new convertible DNA sequence into the full amino acid sequence of a new convertible linker. Two different SfiI restriction sites are underlined and the DNA sequence encoding for the amino acid sequence recognized by HRV-3C protease are boxed.

FIG. 33 illustrates an example of the translation of the new convertible linker sequence into a peptide sequence to efficiently convert the biosensor to a desired target. The sfiI cleavage sites are underlined in the top (A) and shown cleaved in the bottom (B) of the figure. The cleaved nucleotide fragment represents the HRV-3C target site that is removed to generate non-complementary overhang sequences (sticky ends) on the remaining plasmid backbone.

FIG. 34 illustrates an embodiment of a double-stranded oligonucleotide formed from two nucleotides annealed together. The MMP2 protease cleaves the recognition sequence between Tyr-Phe shown by the forward slash symbol, “/”.

FIG. 35 is a graph showing the results (fold increase RFUs over time) of an assay of the activity of recombinant C. botulinum BoNT A Light Chain by three different biosensor proteins expressed from three different genes, SNAP17, SNAP61, and SNAP206 of SNAP25 target sequences for BoNT-A protease.

FIG. 36 shows comparative images of human tissue sections. Panel A shows a biosensor treated tissue section. The arrows point to bright or dull fluorescence from an activated MMP25 biosensor. Panel B shows a control (not MMP25 biosensor) treated tissue section. There is no coloration in panel B. The tissue is fresh human Abdominal Aortic Aneurysm tissue, which has many different proteases in it. The left hand panel, A, shows the results of tissue stained with the MMP25 embodiment of the biosensor described herein. The right hand panel, B, shows a control sensor. The color in Panel A (not visible in the grey scale figures) is the signal from the activated biosensor. The signal is diffuse throughout the collagen bed with small (cell-sized) brighter spots. There is extensive autofluorescence of the tissue. Additional brightness (bright orange/yellow, not visible in grey scale figures) is attributable to paracystalline plaque, which is ubiquitous. The images were collected using an Olympus FV1000 confocal microscope with a 20× oil objective. Importantly, imaging conditions were kept constant (same laser power, photomultiplier tube (pmt) voltage etc.) The samples are semiserial sections.

### DETAILED DESCRIPTION

In the present application, including the claims, other than where otherwise indicated, all numbers expressing quantities, values or characteristics are to be understood as being modified in all instances by the term “about.” Thus, numbers may be read as if preceded by the word “about” even though the term “about” may not expressly appear with the number. Accordingly, unless indicated to the contrary, any numerical parameters set forth in the following description may vary depending on the desired properties one seeks to obtain in the compositions and methods according to the present disclosure. At the very least, and not as an attempt to limit the application of the doctrine of equivalents to the scope of the claims, each numerical parameter described in the present description should at least be construed in light of the number of reported significant digits and by applying ordinary rounding techniques.

Any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The articles “a” and “an” are used herein to refer to one or to more than one (i.e., to at least one) of the grammatical object of the article. By way of example, “an element” means one element or more than one element.

As used herein, the term “amino acid” is intended to embrace all molecules, whether natural or synthetic, which include both an amino functionality and a carboxylate functionality and capable of being included in a poly(amino acid) polymer. Exemplary amino acids include, for example, naturally-occurring amino acids; analogs, derivatives and congeners thereof; amino acid analogs having variant side chains; and all stereoisomers of any of the foregoing.

As used herein, the terms “peptide,” “polypeptide”, and “protein” are synonymous and used interchangeably to refer to a polymer or oligomer of amino acids. In addition, as used herein, the terms “peptide,” “polypeptide”, and “protein” may refer to a discrete sub-unit of a larger peptide construct. As used herein, the term “peptide construct” refers to a peptide comprising discrete peptide domains covalently linked to form the larger peptide construct. The constituent peptides of a peptide construct may be covalently linked through peptide bonds. Any one or more constituent peptides of a peptide construct may also respectively possess an active domain that possesses various activity or functionality, including, but not limited to, receptor-ligand functionality, ligand-target functionality, enzyme-substrate functionality, and antibody-antigen functionality.

As used herein, the term “ligand” refers to a binding moiety for a specific target molecule. The molecule may comprise a cognate receptor, a protein, a small molecule, a hapten, an epitope, or any other relevant molecule. The molecule may comprise an analyte of interest. As used herein, the term “epitope” refers to a structure on a molecule that interacts with another molecule, such as, for example, an antibody or antibody fragment. In various embodiments, epitope refers to a desired region on a target molecule that specifically interacts with another molecule comprising a cognate ligand.

As used herein, “interact” and “interaction” are meant to include detectable interactions between molecules, such as may be detected using, for example, a hybridization assay. The terms “interact” and “interaction” also includes molecular associations including, but not limited to binding and complexation interactions between molecules. Interactions may be, for example, protein-protein, protein-nucleic acid, protein-small molecule or small molecule-nucleic acid, and include for example, antibody-antigen binding, enzyme-substrate binding, receptor-ligand binding, hybridization, and other forms of binding. In various embodiments, an interaction between a ligand and a specific target will lead to the formation of a complex, wherein the ligand and the target are unlikely to dissociate. Such affinity for a ligand and its target can be defined by the dissociation constant (K_{d}) as known in the art. A complex may include a ligand for a specific dye and is referred to herein as a “ligand-dye” complex.

As used herein, the term “antibody” refers to an immunoglobulin, derivatives thereof which maintain specific binding ability, and proteins having a binding domain which is homologous or largely homologous to an immunoglobulin binding domain. As such, an antibody operates as a ligand for its cognate antigen, which can be virtually any molecule. Natural IgG antibodies comprise two heavy chains and two light chains and are bi-valent. The interaction between the variable regions of heavy and light chain forms a binding site capable of specifically binding an antigen. The term “V_{H}” refers herein to a heavy chain variable region of an antibody. The term “V_{L}” refers herein to a light chain variable region of an antibody. Antibodies may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. In various embodiments, antibodies and antibody fragments used with the methods and compositions described herein are derivatives of the IgG class.

As used herein, the term “antibody fragment” refers to any derivative of an antibody which is less than full-length. In various embodiments, the antibody fragment retains at least a significant portion of the full-length antibody's specific binding ability. Examples of antibody fragments include, but are not limited to, Fab, Fab′, F(ab′)₂, Fv, dsFv, scFv, and Fd fragments. The antibody fragment may be produced by any means. For instance, the antibody fragment may be enzymatically or chemically produced by fragmentation of an intact antibody, or it may be recombinantly or partially synthetically produced using genetic engineering methods. An antibody fragment may comprise a single chain antibody fragment. Alternatively, an antibody fragment may comprise multiple chains which are linked together, for instance, by disulfide linkages. An antibody fragment may also comprise a multimolecular complex.

The term “Fab” refers herein to an antibody fragment that is essentially equivalent to that obtained by digestion of immunoglobulin (typically IgG) with the enzyme papain. The heavy chain segment of the Fab fragment is the Fd piece. Such fragments may be enzymatically or chemically produced by fragmentation of an intact antibody, recombinantly produced from a gene encoding the partial antibody sequence, or it may be wholly or partially synthetically produced. Methods for preparing Fab fragments are known in the art. See, for example, Tijssen, Practice and Theory of Enzyme Immunoassays (Elsevier, Amsterdam, 1985).

The term “F(ab′)₂” refers herein to an antibody fragment that is essentially equivalent to a fragment obtained by digestion of an immunoglobulin (typically IgG) with the enzyme pepsin at pH 4.0-4.5. Such fragments may be enzymatically or chemically produced by fragmentation of an intact antibody, recombinantly produced from a gene encoding the partial antibody sequence, or it may be wholly or partially synthetically produced.

The term “Fab′” refers herein to an antibody fragment that is essentially equivalent to that obtained by reduction of the disulfide bridge or bridges joining the two heavy chain pieces in an F(ab′)₂ fragment. Such fragments may be enzymatically or chemically produced by fragmentation of an intact antibody, recombinantly produced from a gene encoding the partial antibody sequence, or it may be wholly or partially synthetically produced.

The term “Fv” refers herein to an antibody fragment that consists of one V_{H} and one V_{L} domain held together by non-covalent interactions. The term “dsFv” is used herein to refer to an Fv with an engineered intermolecular disulfide bond to stabilize the V_{H}-V_{L} pair. Methods for preparing Fv fragments are known in the art. See, for example, U.S. Pat. No. 4,462,334; Hochman et al., Biochemistry, 12, 1130, (1973); Sharon et al., Biochemistry, 15, 1591, (1976); and U.S. Pat. No. 4,355,023.

The terms “single chain antibody,” “single-chain Fv,” and “scFv” refer herein to an antibody fragment comprising the variable light chain (V_{L}) and variable heavy chain (V_{H}) antibody domains covalently connected to one another by a peptide linker moiety. Either the V_{L} or the V_{H} may be the amino-terminal domain. The peptide linker may be of variable length and composition. In various embodiments, peptide linkers may comprise segments of glycine and serine residues, optionally with some glutamic acid or lysine residues interspersed in the peptide linker sequence. Methods for preparing scFvs are known in the art. See, for example, International Application No. PCT/US/87/02208 and U.S. Pat. Nos. 4,704,692; 4,946,778, each of which is incorporated by reference herein in its entirety.

The term “single domain antibody” or “Fd” refers herein to an antibody fragment comprising a V_{H} domain or a V_{L} domain that interacts with a given antigen. A given Fd does not comprise both a V_{H} domain and a V_{L} domain. Methods for preparing single domain antibodies are known in the art. See, for example, Ward et al., Nature, 341:644-646 (1989) and EP0368684.

As used herein, the term “fluorogen” refers to a chemical moiety that exhibits fluorogenic properties. Fluorogens include, but are not limited to, fluorogenic dyes, such as, for example, thiazole orange, malachite green, dimethyl indol red, and derivatives thereof. Not wishing to be bound by theory, the fluorogenic properties of dyes such as, for example, thiazole orange, malachite green, dimethyl indol red, and derivatives thereof are believed to be due to an environmentally sensitive conformational relaxation pathway (Magde et al., Chem. Phys. Letters, 24, 144-148, (1974); Duxbury, Chem. Rev., 93, 381-433, (1993); Furstenberg et al., JACS, 128, 7661-7669, (2006); Silvia et al., JACS, 129, 5710-5718, (2007).

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “DNA fragment” or “nucleotide fragment” refers to any derivative of a nucleic acid which is less than full-length.

The term “restriction enzyme” as used herein means restriction endonucleases that recognize specific base sequences in DNA and cleave the DNA strands at consistent specific locations. Restriction enzymes, in nature, serve a protective function by cleaving foreign DNA. In biotechnology applications, restriction enzymes are used to cleave DNA molecules into specific fragments for ease of analysis and manipulation.

The term “overhang sequences” as used herein means single-stranded nucleic acid sequences left at the ends of double stranded nucleic acid molecules by restriction enzymes, sometimes informally referred to as “sticky ends”.

The term “complementary” as used herein with respect to nucleotide sequences means nucleotide polymers capable of forming double stranded nucleic acid by Watson-Crick base pairing (A base pairs with T; G base pairs with C).

In solution, excitation of fluorogenic dyes with visible light may cause them to undergo rotation and/or torsion around one or more constituent intramolecular bonds. This may result in non-radiative decay of the exited state molecules back to the ground state. Therefore, fluorogenic dyes tend to exhibit very low fluorescence levels in solution. However, when a fluorogenic dye molecule is conformationally constrained, such as, for example, in the active domain of a cognate protein or peptide, the rotational and/or torsional molecular motion induced by visible excitation may be inhibited. As a result, the excitation energy may be given off radiatively when a fluorogenic dye relaxes to the ground state energy level while interacting with a cognate moiety. Examples of fluorogens that find utility in the embodiments disclosed herein are described in International Application Nos. PCT/US2003/029289 and PCT/US2008/051962; and U.S. Application Nos. 60/418,834; 11/077,999; 60/897,120; and 61/013,098, each of which is incorporated by reference herein in its entirety.

As used herein, in reference to fluorescence, the terms “modulate” and “modulation” refer to a change in fluorescence signal intensity, fluorescence lifetime, fluorescence wavelength, or any other measurable property of a fluorescing moiety.

The term “sequence homology” refers to the proportion of base matches between two nucleic acid sequences or the proportion of amino acid matches between two amino acid sequences. When sequence homology is expressed as a percentage, e.g., 50%, the percentage denotes the proportion of matches over the length of sequence from a desired sequence (e.g., SEQ. ID NO: 1) that is compared to some other sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occur in both sequences to yield the number of matched positions, dividing the number of matched positions by the total number of positions in the comparison window, and multiplying the result by 100 to yield the percentage of sequence identity. Methods to calculate sequence identity are known to those of skill in the art.

A selectivity component may be any molecule which is capable of selectively interacting with a desired target molecule, including an antibody or antibody fragment. For example, selectivity components may be monoclonal antibodies, or derivatives or analogs thereof, including without limitation: Fv fragments and single chain Fv (scFv) fragments. In certain embodiments, a biosensor may comprise a selectivity component having at least about 85% sequence identity with a sequence within SEQ ID NO:21 through 40. In certain other embodiments, an isolated, purified biosensor may comprise a selectivity component having at least about 85% sequence identity with a sequence within SEQ ID NO:21 through 40. In certain embodiments, a vector may comprise a nucleic acid sequence having at least about 85% sequence identity to a polynucleotide encoding a protein with the appropriate sequence corresponding to SEQ ID NO:21 through 40. In certain other embodiments, a vector may comprise a nucleic acid sequence having at least about 95% sequence identity to a polynucleotide encoding a protein with the appropriate sequence corresponding to SEQ ID NO:21 through 40.

Various embodiments disclosed herein are directed to linked peptide fluorogenic biosensors. The disclosed biosensors comprise a peptide construct. The peptide construct may comprise a fluorogen-activating peptide and a blocking peptide. The fluorogen-activating peptide comprises an active domain that specifically interacts with a fluorogen to modulate the fluorescence signal produced by the fluorogen. The fluorogen-activating peptide is linked to the blocking peptide through a peptide linker.

The peptide linker may comprise an amino acid sequence that is specifically recognized as a modification substrate by a cognate enzyme. The blocking peptide may specifically associate with the fluorogen-activating peptide, thereby blocking the active domain of the fluorogen-activating peptide, when the peptide linker is in an unmodified state. The fluorogen-activating peptide and the blocking peptide may at least partially disassociate when the peptide linker is modified by a cognate enzyme, thereby allowing the fluorogen-activating peptide to interact with a cognate fluorogen and modulate a fluorescence signal.

In various embodiments, the present disclosure is directed to biosensors comprising a fluorogen-activating peptide comprising a variable domain of an antibody, and a blocking peptide comprising a variable domain of an antibody. One of the fluorogen-activating peptide and the blocking peptide may comprise a variable heavy chain domain of an antibody and the other peptide may comprise a variable light chain domain of a different antibody. The fluorogen-activating peptide may comprise a single domain antibody. The blocking peptide may be linked to the fluorogen-activating peptide through a peptide linker comprising an amino acid sequence that is specifically recognized as a cleavage substrate by a cognate protease. The blocking peptide may associate with the fluorogen-activating peptide thereby blocking the active domain of the fluorogen-activating peptide when the linker is intact. The fluorogen-activating peptide and the blocking peptide may at least partially disassociate when the linker is cleaved by a cognate protease, thereby allowing the fluorogen-activating peptide to interact with a cognate fluorogen and modulate a fluorescence signal.

In various embodiments, the present disclosure is directed to biosensors comprising a fluorogen-activating peptide comprising a variable domain of an antibody, a blocking peptide comprising a variable domain of an antibody, and a phospho(amino acid) binding peptide linked to the fluorogen-activating peptide or the blocking peptide. One of the fluorogen-activating peptide and the blocking peptide may comprise a variable heavy chain domain of an antibody and the other peptide may comprise a variable light chain domain of a different antibody. The fluorogen-activating peptide may comprise a single domain antibody. The blocking peptide may be linked to the fluorogen-activating peptide through a peptide linker comprising an amino acid sequence that may be specifically recognized as a phosphorylation substrate by a cognate protein kinase. The blocking peptide may associate with the fluorogen-activating peptide thereby blocking the active domain of the fluorogen-activating peptide when the peptide linker is not phosphorylated. The fluorogen-activating peptide and the blocking peptide may at least partially disassociate when the peptide linker is phosphorylated by a cognate protein kinase, thereby allowing the fluorogen-activating peptide to interact with a cognate fluorogen and produce a fluorescence signal.

In various embodiments, the present disclosure is directed to biosensors comprising a fluorogen-activating peptide comprising a variable domain of an antibody, a blocking peptide comprising a variable domain of an antibody, and a bromo-domain peptide that is linked to the fluorogen-activating peptide or the blocking peptide. One of the fluorogen-activating peptide and the blocking peptide may comprise a variable heavy chain domain of an antibody and the other peptide may comprise a variable light chain domain of a different antibody. The fluorogen-activating peptide may comprise a single domain antibody. The blocking peptide may be linked to the fluorogen-activating peptide through a peptide linker comprising an amino acid sequence that may be specifically recognized as an acetylation substrate by a cognate acetyltransferase. The blocking peptide may associate with the fluorogen-activating peptide thereby blocking the active domain of the fluorogen-activating peptide when the peptide linker is not acetylated. The fluorogen-activating peptide and the blocking peptide may at least partially disassociate when the peptide linker is acetylated by a cognate acetyltransferase, thereby allowing the fluorogen-activating peptide to interact with a cognate fluorogen and produce a fluorescence signal.

In various embodiments, the present disclosure is directed to a composition comprising a fluorogen and a biosensor as disclosed herein. Fluorogens finding utility in the compositions disclosed herein include, but are not limited to, thiazole orange, malachite green, dimethyl indole red, and derivatives thereof. In various embodiments, the present disclosure is directed to methods for analyzing enzyme activity. The disclosed methods may comprise contacting a medium comprising an analyte enzyme with a composition comprising a fluorogen and a biosensor as disclosed herein, and detecting a fluorescence signal produced by an interaction between a fluorogen-activating peptide of the biosensor construct and the fluorogen.

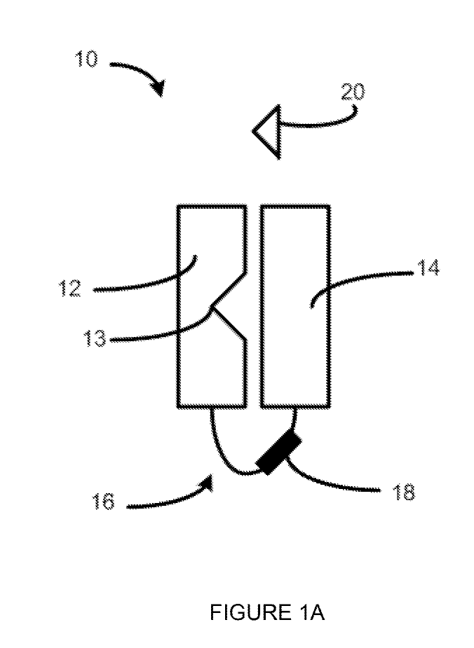

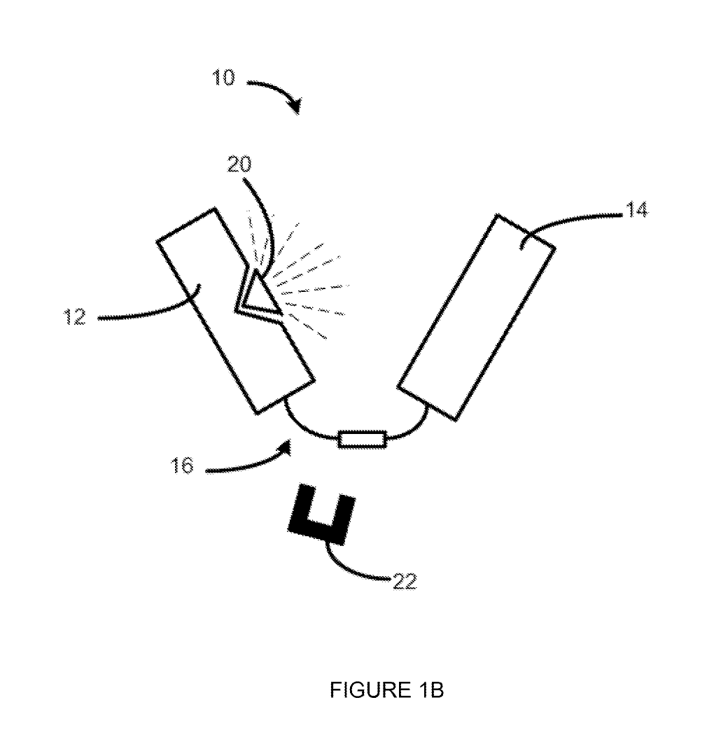

FIGS. 1A and 1B illustrate a biosensor according to various embodiments disclosed herein. Biosensor 10 comprises a fluorogen-activating peptide 12 having an active domain 13 that is capable of specifically interacting with a cognate fluorogen 20 to modulate a fluorescence signal produced by the fluorogen. The fluorogen-activating peptide 12 is linked to a blocking peptide 14 through a peptide linker 16. The peptide linker 16 comprises an amino acid sequence 18 that is specifically recognized as a modification substrate by a cognate enzyme 22. As illustrated in FIG. 1A, the blocking peptide 14 associates with the fluorogen-activating peptide 12 thereby blocking the active domain 13 of the fluorogen-activating peptide 12 when the peptide linker 16 is in an unmodified state. As illustrated in FIG. 1B, the fluorogen-activating peptide 12 and the blocking peptide 14 at least partially disassociate when the linker 16 is modified by a cognate enzyme 22, thereby allowing the fluorogen-activating peptide 12 to interact with the cognate fluorogen 20 and modulate a fluorescence signal.

In various embodiments, the peptide linker may be a synthetic flexible chain of from 15 amino acids in length to 30 amino acids in length. The peptide linker may comprise relatively small amino acid residues, including, but not limited to, glycine. Small amino acid residues may reduce the steric bulk and increase the flexibility of the peptide linker. The peptide linker may also comprise polar amino acids, including, but not limited to, serine. Polar amino acid residues may increase the aqueous solubility of the peptide linker. In various embodiments, the peptide linker may comprise an amino acid sequence comprising (Gly₄Ser)₃ (SEQ ID NO:1), and an amino acid sequence that is specifically recognized as a modification substrate by a cognate enzyme. In various embodiments, the peptide linker may comprise a site-specific modification amino acid sequence located on the N-terminal end of a (Gly₄Ser)₃ sequence; and in various embodiments the peptide linker may comprise a site-specific modification amino acid sequence located on the C-terminal end of a (Gly₄Ser)₃ sequence.

In various embodiments, the disclosed biosensors may comprise a peptide linker that comprises a site-specific protease recognition amino acid sequence (i.e., an amino acid sequence that is specifically recognized as a proteolysis substrate by a cognate protease). As used herein, the term “protease” refers to an enzyme involved in proteolysis, that is, catabolic hydrolysis of peptide bonds that link amino acids together in peptide chains. A peptide linker comprising a site-specific protease recognition amino acid sequence as a modification substrate may be cleaved by a cognate protease.

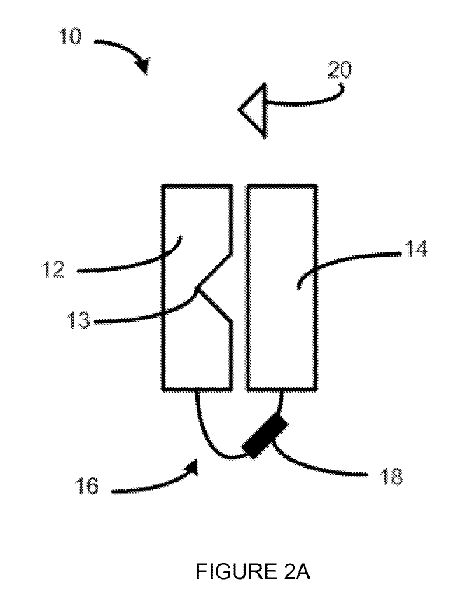

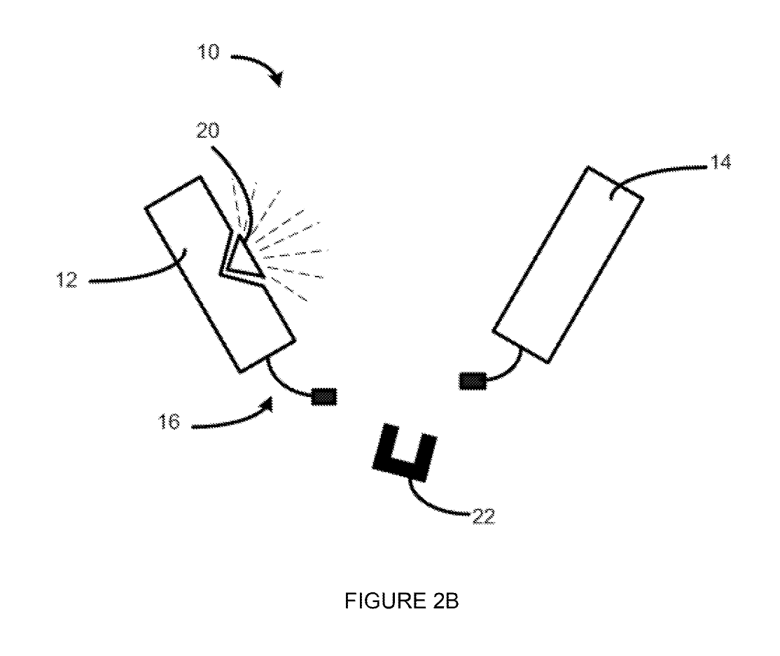

In various embodiments disclosed herein, when a protease recognizes a site-specific amino acid sequence contained in a peptide linker, the peptide linker may be cleaved, thereby breaking the covalent linkage between the fluorogen-activating peptide and the blocking peptide. The fluorogen-activating peptide and the blocking peptide may then at least partially dissociate or completely dissociate and diffuse away from each other. Not wishing to be bound by theory, the at least partial dissociation may be driven at least in part by an increase in translational entropy. When the fluorogen-activating peptide is at least partially disassociated from the blocking peptide, the active domain of the fluorogen-activating peptide may become un-blocked, and therefore, may become free to interact with a cognate fluorogen and modulate a fluorescence signal produced by the fluorogen. FIGS. 2A and 2B illustrate a biosensor according to this embodiment.

In various embodiments, the peptide linker may comprise a site-specific protease recognition amino acid sequence specifically recognized as a cleavage site by a protease selected from the group consisting of a serine protease, a threonine protease, a cysteine protease, an aspartic acid protease, a matrix metalloproteinase, and a glutamic acid protease. In various embodiments, the peptide linker may comprise a site-specific protease recognition amino acid sequence specifically recognized as a cleavage substrate by a protease selected from the group consisting of furan protease, tobacco etch virus (“TEV”) protease, a 3C protease, a caspase, and a matrix metalloproteinase, for example.

In various embodiments, the disclosed peptide linker may comprise an amino acid sequence specifically recognized as a cleavage site by furin. Furin is a protease that is involved in a protein secretory pathway in eukaryotic cells. In mammalian cells, for example, furin is localized to the protein secretory pathway between the trans-Golgi network and the cell surface. A consensus recognition sequence for furin protease has been reported as Arg-Xaa-(Lys/Arg)-Arg-Ser. By way of example, furin protease may specifically recognize a short recognition sequence (e.g., Arg-Lys-Lys-Arg-Ser) or a long recognition sequence (e.g., Asn-Ser-Arg-Lys-Lys-Arg-Ser-Thr-Ser-Ala). In various embodiments, the disclosed peptide linker may comprise an amino acid sequence comprising Arg-Xaa-(Lys/Arg)-Arg-Ser. In various embodiments, the disclosed peptide linker may comprise an amino acid sequence comprising Arg-Lys-Lys-Arg-Ser (SEQ ID NO:3). In various embodiments, the disclosed peptide linker may comprise an amino acid sequence comprising Asn-Ser-Arg-Lys-Lys-Arg-Ser-Thr-Ser-Ala (SEQ ID NO:5).

In various embodiments, the disclosed peptide linker may comprise an amino acid sequence specifically recognized as a cleavage site by TEV protease. TEV protease is a site-specific cysteine protease that is found in the tobacco etch virus. TEV protease is used, for example, to remove affinity tags from purified proteins. A consensus recognition sequence for TEV protease has been reported as Glu-Asn-Leu-Tyr-Phe-Gln-Gly, with cleavage occurring between the Gln and Gly residues. In various embodiments, the disclosed peptide linker may comprise an amino acid sequence comprising Glu-Asn-Leu-Tyr-Phe-Gln-Gly (SEQ ID NO:7).

In various embodiments, the disclosed peptide linker may comprise an amino acid sequence specifically recognized as a cleavage site by a 3C protease. 3C proteases are viral enzymes that cleave viral precursor polyproteins to form functional proteins, and are thought to be involved in viral replication. A consensus recognition sequence for human rhinovirus 3C (“HRV-3C”) protease, for example, has been reported as Leu-Glu-Val-Leu-Phe-Gln-Gly-Pro, with cleavage occurring between the Gln and Gly residues. In various embodiments, the disclosed peptide linker may comprise an amino acid sequence comprising Leu-Glu-Val-Leu-Phe-Gln-Gly-Pro (SEQ ID NO:9).

In various embodiments, the disclosed peptide linker may comprise an amino acid sequence specifically recognized as a cleavage site by a caspase. Caspases (cysteine-aspartic acid proteases) are a family of cysteine proteases thought to be involved in apoptosis, necrosis and inflammation, for example. Eleven caspases have been identified in humans. A consensus recognition sequence for caspase 1, for example, has been reported as Tyr-Val-Ala-Asp. A consensus recognition sequence for caspase 3, for example, has been reported as Asp-Glu-Val-Asp. In various embodiments, the disclosed peptide linker may comprise an amino acid sequence comprising the sequence Tyr-Val-Ala-Asp (SEQ ID NO:11). In various embodiments, the disclosed peptide linker may comprise an amino acid sequence comprising the sequence Asp-Glu-Val-Asp (SEQ ID NO:13).

In various embodiments, the disclosed peptide linker may comprise an amino acid sequence specifically recognized as a cleavage site by a matrix metalloproteinase. Matrix metalloproteinases (“MMPs”) are zinc-dependent proteases capable of cleaving a number of extracellular matrix proteins and cell surface receptors, for example. MMPs are thought to be involved in the release of apoptotic ligands and chemokine activation/inactivation. MMPs are also thought to be involved in cell proliferation, migration (adhesion/dispersion), differentiation, angiogenesis, and host defense, for example. A number of MMPs have been identified. A consensus recognition sequence for MMP25, for example, has been reported as Val-Met-Arg-Leu-Val-Val. In various embodiments, the disclosed peptide linker may comprise an amino acid sequence comprising Val-Met-Arg-Leu-Val-Val (SEQ ID NO:15).

In various embodiments, the disclosed biosensors may comprise a peptide linker comprising a site-specific kinase recognition amino acid sequence (i.e., an amino acid sequence that is specifically recognized as a phosphorylation substrate by a cognate protein kinase). As used herein, the term “kinase” refers to an enzyme involved in phosphorylation, that is, enzymatic transfer of phosphate groups from donor molecules (e.g., ATP) to specific target substrates. As used herein, the terms “kinase” and “phosphotransferase” are synonymous. As used herein, the term “protein kinase” refers to a kinase that recognizes a site-specific amino acid sequence and phosphorylates a peptide comprising such a recognition sequence. A peptide linker comprising a site-specific kinase recognition amino acid sequence as a modification substrate may be phosphorylated by a cognate kinase.

In various embodiments disclosed herein, when a kinase recognizes a site-specific amino acid sequence contained in a peptide linker, the peptide linker may be phosphorylated, thereby modifying the linkage between the fluorogen-activating peptide and the blocking peptide. The modification of the linker may change the chemical and physical conditions within the microenvironment surrounding the peptide linker. As used herein, the term “microenvironment” refers to localized conditions within a larger area. For example, modification of a peptide sequence may alter the local chemical and/or physical conditions surrounding the peptide sequence, which may result in a conformational change in the intramolecular secondary or tertiary structure of a peptide construct comprising the peptide sequence. In this regard, the microenvironment surrounding the peptide sequence may be changed when the peptide sequence is modified.

A conformational change in a peptide construct according to the disclosed embodiments may result in at least partial dissociation between a fluorogen-activating peptide and a blocking peptide. Not wishing to be bound by theory, the at least partial dissociation may be driven at least in part by a change in the chemical and/or physical conditions in the microenvironment surrounding the phosphorylated peptide linker. When the fluorogen-activating peptide is at least partially disassociated from the blocking peptide, the active domain of the fluorogen-activating peptide may become un-blocked, and therefore, may become free to interact with a cognate fluorogen and modulate a fluorescence signal. Referring to FIGS. 1A and 1B, enzyme 22 may be a kinase that recognizes amino acid sequence 18 and phosphorylates peptide linker 16. As a result, the change in the microenvironment surrounding the peptide linker 16 may induce at least partial dissociation between the fluorogen-activating peptide 12 and the blocking peptide 14.

In various embodiments, the disclosed peptide linker may comprise an amino acid sequence specifically recognized as a phosphorylation substrate by protein kinase A (“PKA”). PKA is a cAMP-dependent kinase involved in numerous parallel signaling networks and pathways. A consensus recognition sequence for PKA, for example, has been reported as Leu-Arg-Arg-Ala-Ser-Leu-Gly (also known as PKA kemptide phosphorylation sequence). Various modified PKA kemptide phosphorylation sequences are also known to be specifically-recognized by PKA, for example, the amino acid sequences Leu-Arg-Arg-Ala-Ser-Leu-Pro and Leu-Leu-Arg-Arg-Ala-Ser-Leu-Gly-Pro (SEQ ID NO:17). In various embodiments, the disclosed peptide linker may comprise an amino acid sequence comprising a Leu-Arg-Arg-Ala-Ser-Leu-Gly sequence; a Leu-Arg-Arg-Ala-Ser-Leu-Pro sequence; or a Leu-Leu-Arg-Arg-Ala-Ser-Leu-Gly-Pro sequence.

In various embodiments, a biosensor comprising a peptide linker comprising a site-specific kinase recognition amino acid sequence may further comprise a phospho(amino acid) binding peptide linked to the fluorogen-activating peptide or the blocking peptide. As used herein, the term “phospho(amino acid) binding peptide” refers to a peptide comprising a domain that specifically interacts with another peptide comprising a phosphorylated amino acid. For example, a phospho(amino acid) binding peptide may preferentially complex with a peptide comprising a phosphorylated amino acid. A peptide construct comprising a fluorogen-activating peptide and a blocking peptide connected through a peptide linker comprising a site-specific kinase recognition amino acid sequence, and a phospho(amino acid) binding peptide linked to the fluorogen-activating peptide or the blocking peptide, may exhibit a conformational change when the peptide linker is phosphorylated by a kinase. Not wishing to be bound by theory, the intramolecular interaction between the phosphorylated peptide linker and the phospho(amino acid) binding peptide may substantially change the orientation of the peptide construct such that the fluorogen-activating peptide and the blocking peptide at least partially disassociate.

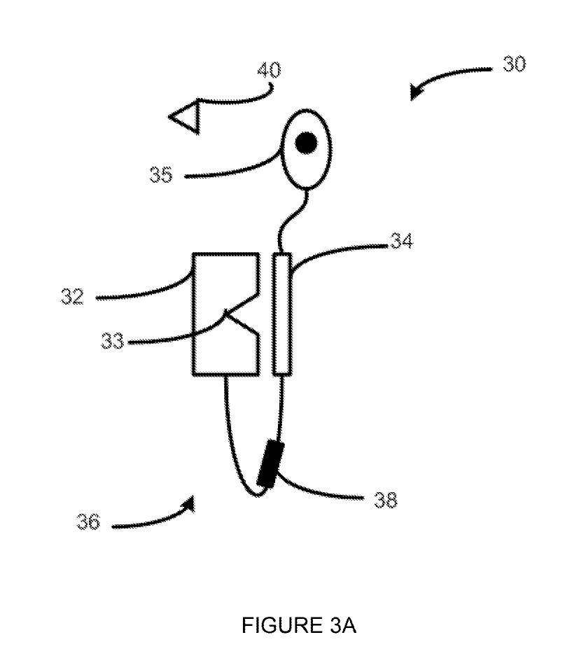

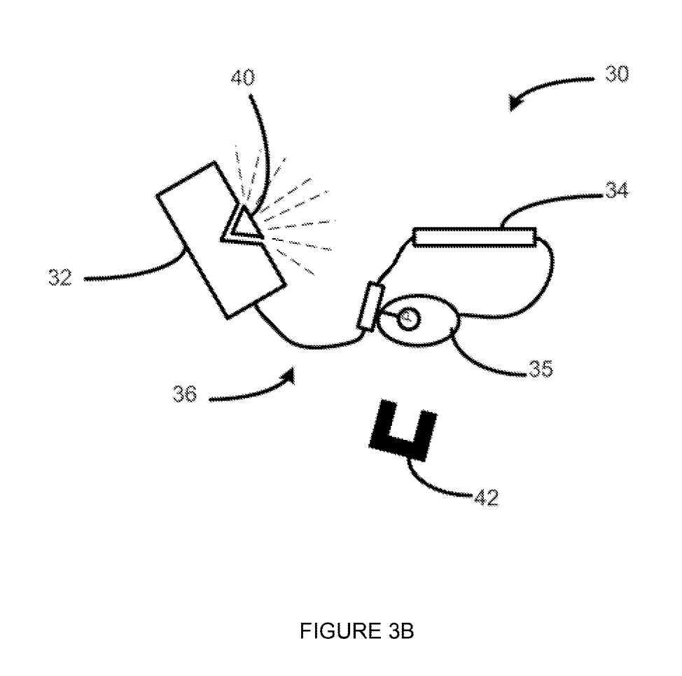

FIGS. 3A and 3B illustrate a biosensor according to the embodiments disclosed herein. Biosensor 30 comprises a fluorogen-activating peptide 32 having an active domain 33 that is capable of specifically interacting with a cognate fluorogen 40 to modulate a fluorescence signal produced by the fluorogen. The fluorogen-activating peptide 32 is linked to a blocking peptide 34 through a peptide linker 36. The peptide linker 36 comprises a site-specific kinase recognition amino acid sequence 38 that is specifically recognized by a cognate kinase 42. As illustrated in FIG. 3A, the blocking peptide 34 associates with the fluorogen-activating peptide 32 thereby blocking the active domain 33 of the fluorogen-activating peptide 32 when the peptide linker 36 is not phosphorylated.

As illustrated in FIG. 3B, when kinase 42 phosphorylates peptide linker 36, the peptide linker 36 interacts with a phospho(amino acid) binding peptide 35. This interaction induces a conformational change in the peptide construct resulting in at least partial dissociation between the fluorogen-activating peptide 32 and the blocking peptide 34, thereby allowing the fluorogen-activating peptide 32 to interact with the cognate fluorogen 40 and modulate a fluorescence signal.

In various embodiments, the disclosed biosensors may comprise a peptide construct comprising a phospho(amino acid) binding peptide, a fluorogen-activating peptide, a peptide linker comprising a site-specific kinase recognition amino acid sequence, and a blocking peptide. In various embodiments, the phospho(amino acid) binding peptide is linked to the fluorogen-activating peptide. In various embodiments, the phospho(amino acid) binding peptide is linked to the blocking peptide. In various embodiments, the phospho(amino acid) binding peptide comprises 14-3-3τ protein. 14-3-3τ protein is a phospho-serine binding protein which recognizes and interacts with phosphorylated serine amino acid residues in peptides (Zhang et al., Proc. Natl. Acad. Sci. USA, 98, 14997-15002, 2001, which is incorporated by reference herein in its entirety).

In various embodiments, the disclosed biosensors comprise a peptide linker that comprises a site-specific acetyltransferase recognition amino acid sequence (i.e., an amino acid sequence that is specifically recognized as an acetylation substrate by a cognate acetyltransferase). As used herein, the term “acetyltransferase” refers to an enzyme involved in acetylation, that is, enzymatic transfer of acetyl groups from donor molecules (e.g., acetyl CoA) to specific target substrates. As used herein, the term “acetyltransferase” also refers to an enzyme that recognizes a site-specific amino acid sequence and acetylates a peptide comprising such a recognition sequence. A peptide linker comprising a site-specific acetyltransferase recognition amino acid sequence as a modification substrate may be acetylated by a cognate acetyltransferase.

In various embodiments disclosed herein, when an acetyltransferase recognizes a site-specific amino acid sequence contained in a peptide linker, the peptide linker may be acetylated, thereby modifying the linkage between the fluorogen-activating peptide and the blocking peptide. The modification of the linker may change the chemical and physical conditions within the microenvironment surrounding the peptide linker. The acetylation of a peptide sequence may alter the local chemical and/or physical conditions surrounding the peptide sequence, which may result in a conformational change in the intramolecular secondary or tertiary structure of a peptide construct comprising a peptide sequence. In this regard, the microenvironment surrounding a peptide sequence may be changed when the peptide sequence is acetylated.

A conformational change in a peptide construct according to the disclosed embodiments may result in at least partial dissociation between the fluorogen-activating peptide and the blocking peptide. Not wishing to be bound by theory, the at least partial dissociation may be driven at least in part by a change in the chemical and/or physical conditions in the microenvironment surrounding the acetylated peptide linker. When the fluorogen-activating peptide is at least partially disassociated from the blocking peptide, the active domain of the fluorogen-activating peptide may become un-blocked, and therefore, may become free to interact with a cognate fluorogen and modulate a fluorescence signal. Referring to FIGS. 1A and 1B, enzyme 22 may be an acetyltransferase that recognizes amino acid sequence 18 and acetylates peptide linker 16. As a result, the change in the microenvironment surrounding the peptide linker 16 induces at least partial dissociation between the fluorogen-activating peptide 12 and the blocking peptide 14.

In various embodiments, the disclosed peptide linker may comprise an amino acid sequence specifically recognized as an acetylation substrate by a histone acetyltransferase (“HAT”). For example, acetylation of histone H3 lysine 56 (“H3-K56”) is reported to be mediated by HATs that recognize the amino acid sequence Ile-Arg-Arg-Phe-Gln-Lys-Ser-Thr-Asp-Leu-Leu. In various embodiments, the disclosed peptide linker may comprise an amino acid sequence comprising Ile-Arg-Arg-Phe-Gln-Lys-Ser-Thr-Asp-Leu-Leu (SEQ ID NO:19).

In various embodiments, a biosensor comprising a peptide linker comprising a site-specific acetyltransferase recognition amino acid sequence may further comprise an acetyl(amino acid) binding peptide linked to the fluorogen-activating peptide or the blocking peptide. As used herein, the term “acetyl(amino acid) binding peptide” refers to a peptide comprising a domain that specifically interacts with a peptide comprising an acetylated amino acid. For example, an acetyl(amino acid) binding peptide may preferentially complex with a peptide comprising an acetylated amino acid. A peptide construct comprising a fluorogen-activating peptide and a blocking peptide connected through a peptide linker comprising a site-specific acetyltransferase recognition amino acid sequence, and an acetyl(amino acid) binding peptide linked to either the fluorogen-activating peptide or the blocking peptide, may exhibit a conformational change when the peptide linker is acetylated by an acetyltransferase. Not wishing to be bound by theory, the intramolecular interaction between the acetylated peptide linker and the acetyl(amino acid) binding peptide may substantially change the orientation of the peptide construct such that the fluorogen-activating peptide and the blocking peptide at least partially disassociate.

A biosensor according to this embodiment may function analogously to the biosensors illustrated in FIGS. 3A and 3B comprising a peptide linker comprising a site-specific kinase recognition amino acid sequence and a phospho(amino acid) binding peptide. In this embodiment, the interaction would occur between a site-specific acetyltransferase recognition amino acid sequence and an acetyl(amino acid) binding peptide. This interaction may induce a conformational change in the peptide construct resulting in at least partial dissociation between the fluorogen-activating peptide and the blocking peptide, thereby allowing the fluorogen-activating peptide to interact with the cognate fluorogen and modulate a fluorescence signal.

In various embodiments, the disclosed biosensors may comprise a peptide construct comprising an acetyl(amino acid) binding peptide, a fluorogen-activating peptide, a peptide linker comprising a site-specific acetyltransferase recognition amino acid sequence, and a blocking peptide. In various embodiments, the acetyl(amino acid) binding peptide is linked to the fluorogen-activating peptide. In various embodiments, the acetyl(amino acid) binding peptide is linked to the blocking peptide. In various embodiments, the acetyl(amino acid) binding peptide comprises a bromo-domain protein. Bromo-domain proteins are acetyl-lysine binding proteins which recognize and interact with acetylated lysine amino acid residues in peptides (Mujtaba et al, Oncogene, 26, 5521-5527, 2007, which is incorporated by reference herein in its entirety).

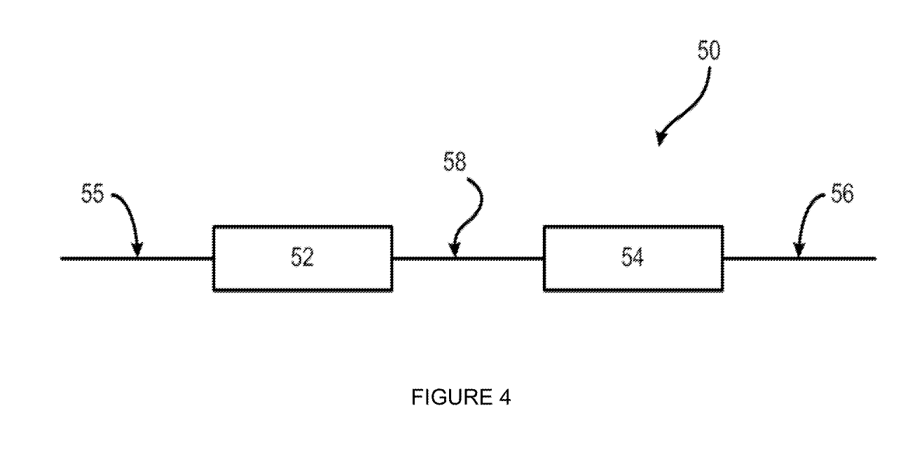

In various embodiments, the disclosed biosensors may comprise a peptide construct having a linear peptide structure as illustrated in FIG. 4. Peptide construct 50 may comprise peptide regions 52, 54, 55, 56 and 58. Peptide region 52 may comprise a fluorogen-activating peptide and peptide region 54 may comprise a blocking peptide. Peptide regions 52 and 54 are linked by a peptide region 58, which may comprise a peptide linker. In various embodiments, peptide region 58 may comprise a site-specific kinase recognition amino acid sequence or a site-specific acetyltransferase recognition amino acid sequence, and either peptide region 55 or peptide region 56 may comprise a phospho(amino acid) binding peptide or an acetyl(amino acid) binding peptide. In various embodiments, peptide region 58 may comprise a phospho(amino acid) binding peptide or an acetyl(amino acid) binding peptide, and peptide region 55 and/or peptide region 56 may comprise either a site-specific kinase recognition amino acid sequence or a site-specific acetyltransferase recognition amino acid sequence. In various embodiments, peptide region 55 may comprise a phospho(amino acid) binding peptide or an acetyl(amino acid) binding peptide, and peptide region 56 may comprise either a site-specific kinase recognition amino acid sequence or a site-specific acetyltransferase recognition amino acid sequence.

Accordingly, a site-specific kinase recognition amino acid sequence or a site-specific acetyltransferase recognition amino acid sequence may be located in peptide region 55, 56 or 58, and a phospho(amino acid) binding peptide or an acetyl(amino acid) binding peptide may be located in peptide region 55, 56 or 58, provided however, that the kinase or acetyltransferase recognition substrate is not located in the same peptide region as a binding peptide. Thus, the biosensors disclosed herein are not limited to a construction wherein a peptide linker comprises an enzyme recognition sequence, and a binding peptide is located at the end of a fluorogen-activating peptide or blocking peptide, opposite the peptide linker. In various embodiments, the biosensors disclosed herein comprise a peptide linker comprising a binding peptide, and a site-specific enzyme recognition sequence located at an end of a fluorogen-activating peptide and/or a blocking peptide, opposite the peptide linker. In various embodiments, the biosensors disclosed herein comprise a site-specific enzyme recognition sequence located at an end of a fluorogen-activating peptide or a blocking peptide, opposite the peptide linker, and a binding peptide located at the opposite end of the peptide construct.

In various embodiments, the disclosed biosensors may function in a reversible manner. For example, biosensors comprising a fluorogen-activating peptide, a blocking peptide, a peptide linker comprising a site-specific kinase recognition amino acid sequence, and, optionally, a phospho(amino acid) binding peptide may function as biosensors for kinase and phosphatase activity. As a kinase biosensor, the peptide constructs according to embodiments disclosed herein may undergo a conformational change as a result of phosphorylation of the peptide linker, which may result in an at least partial disassociation between the fluorogen-activating peptide and the blocking peptide, which may result in an increase in fluorescence produced by an interaction between a fluorogen and the fluorogen-activating peptide.

In various embodiments, a kinase-activated (i.e., phosphorylated) biosensor may comprise a complex between a fluorogen molecule and the fluorogen-activating peptide. If this complex comes into contact with a phosphatase enzyme, the complex may be de-phosphorylated. The de-phosphorylation may cause the peptide construct to revert back to its original conformation, which may disrupt the interaction between the fluorogen and the fluorogen-activating peptide. This may result in a decrease in fluorescence. Thus, a phosphorylated biosensor according to various embodiments described herein may function as a phosphatase biosensor.

In addition, as an acetyltransferase biosensor, the peptide constructs according to embodiments disclosed herein may undergo a conformational change as a result of acetylation of the peptide linker, which may result in an at least partial disassociation between the fluorogen-activating peptide and the blocking peptide, which may result in an increase in fluorescence produced by an interaction between a fluorogen and the fluorogen-activating peptide.

In various embodiments, an acetyltransferase-activated (i.e., phosphorylated) biosensor may comprise a complex between a fluorogen molecule and the fluorogen-activating peptide. If this complex comes into contact with a de-acetylase enzyme, the complex may be de-acetylated. The de-acetylation may cause the peptide construct to revert back to its original conformation, which may disrupt the interaction between the fluorogen and the fluorogen-activating peptide. This may result in a decrease in fluorescence. Thus, an acetylated biosensor according to various embodiments described herein may function as a de-acetylase biosensor.

In various embodiments, the fluorogen-activating peptide and the blocking peptide may comprise an antibody or antibody fragment. Examples of antibody fragments finding utility in the disclosed embodiments include, but are not limited to, Fab, Fab′, F(ab′)₂, Fv, dsFv, scFv, and Fd fragments. In various embodiments, the fluorogen-activating peptide and the blocking peptide may comprise a variable chain domain (V_{H} or V_{L}) of an antibody. In various embodiments, the fluorogen-activating peptide may comprise a variable heavy chain domain (V_{H}) of an antibody and the blocking peptide may comprise a variable light chain domain (V_{L}) of an antibody, and in other embodiments, the fluorogen-activating peptide may comprise a variable light chain domain (V_{L}) of an antibody and the blocking peptide may comprise a variable heavy chain domain (V_{H}) of an antibody. In various embodiments, the fluorogen-activating peptide may comprise a single-chain antibody. In various embodiments, the variable heavy chain domain (V_{H}) and the variable light chain domain (V_{L}) may be derived from different antibodies.

In various embodiments, the variable chain domains (V_{H} or V_{L}) comprising the fluorogen-activating peptide and the blocking peptide may be derived from scFvs. scFvs may be derived by genetic engineering manipulations of antibody DNA. Using genetic engineering techniques known in the art, synthetic scFv genes may be constructed from gene segments coding for the variable domains of the heavy and light chains (V_{H} and V_{L}) covalently linked by a synthetic DNA segment that codes for a peptide linker. The peptide linker may be of sufficient length (for example, 15 or more amino acid residues in length) to allow the V_{H} and V_{L} domains to associate intramolecularly into a characteristic V_{H}/V_{L} conformation found at the antigen binding ends of native antibodies. A complex human scFv library comprising approximately 10⁹ synthetically recombined heavy and light chain variable regions is available in a yeast surface display format. See, for example, Feldhaus et al., Nat. Biotechnol., 21, 163-170, (2003); and Boder et al. Nat. Biotechnol., 15, 553-557, (1997), each of which is incorporated by reference herein in its entirety.

Specific interactions between particular target molecules and particular scFvs may be determined by screening a yeast surface-display scFv library. See, for example, Wittrup et al., Methods Enzymol., 328, 430-444, (2000); Boder et al, Proc. Natl. Acad. Sci. USA, 97, 10701-10705, (2000); and Swers et al. Nucleic Acids Res., 32, e36, (2004), each of which is incorporated by reference herein in its entirety. Particular scFvs that specifically interact with particular fluorogens may be determined, for example, by screening a yeast surface-display scFv library. See, for example, Ozhalici-Unal et al., JACS, 130, 12620-12621, (2008); and Szent-Gyorgyi et al., Nat. Biotechnol., 26, 235-240, (2008), each of which is incorporated by reference herein in its entirety. Other genetic selection methods for screening scFvs for specific interaction with target molecules are known in the art, such as, for example, phage display methods. Phage display systems, their construction and operation, and associated screening methods are described in detail, for example, in U.S. Pat. Nos. 5,702,892; 5,750,373, 5,821,047; 5,948,635; and 6,127,132, each of which is incorporated by reference herein in its entirety.

Examples of scFvs that specifically interact with thiazole orange derivatives and scFvs that specifically interact with malachite green derivatives are described in Szent-Gyorgyi et al., Nat. Biotechnol., 26, 235-240, (2008), and in International Patent Application No. PCT/US2008/051962, each of which is incorporated by reference herein in its entirety. In some embodiments, scFvs require both the V_{H} and V_{L} domains for fluorogen interaction. In other embodiments, only the V_{H} domain or the V_{L} domain, alone, is necessary for a scFv to specifically interact with a fluorogen and modulate the fluorescence signal produced by the fluorogen. In these embodiments, the associated partner domain may contribute nothing to (or in fact inhibit) interaction between the scFv and a cognate fluorogen. Functional single-domain (either V_{H} or V_{L}) scFvs may interact with cognate fluorogens with a high degree of affinity and specificity without an associated partner domain (either V_{L} or V_{H}, respectively). In these embodiments, the fluorogen-activating variable domain may be described as a single domain antibody.

In some embodiments, a functional V_{H} or V_{L} domain may be paired with a non-functional partner domain comprising a V_{H} or V_{L} domain from a different antibody (or different scFv). In these embodiments, if the functional fluorogen-interacting domain is a V_{H} domain, then the non-functional partner domain may be a V_{L} domain. If the functional fluorogen-interacting domain is a V_{L} domain, then the non-functional partner domain may be a V_{H} domain. The functional domain and the non-functional partner domain may be covalently linked through a peptide linker, thereby forming a peptide construct comprising a synthetic hybrid scFv structure.

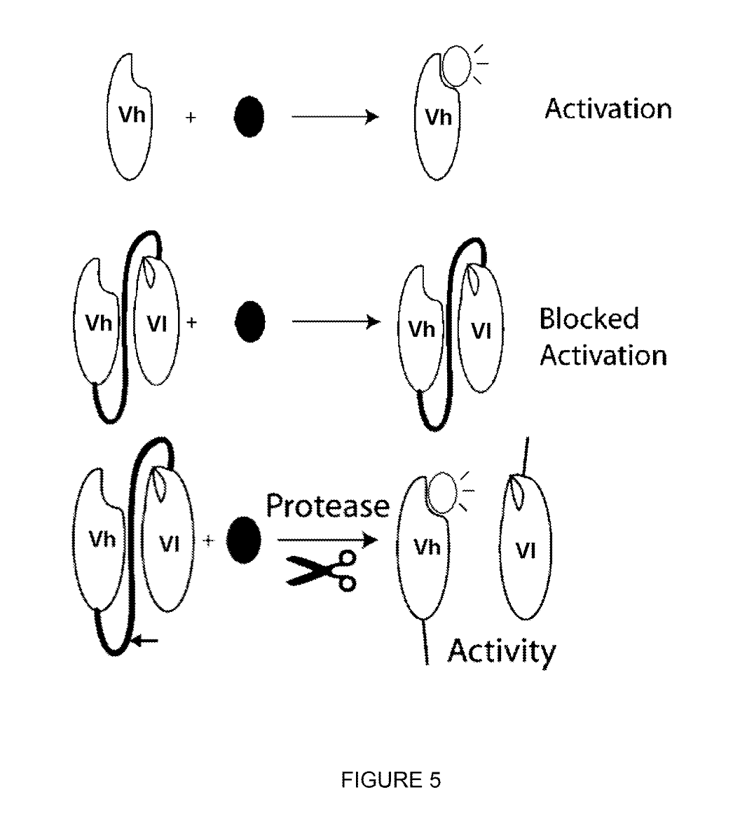

The non-functional partner domain may associate with the functional domain when covalently linked through a peptide linker. The association may partially or totally block the active portion of the functional domain, which may interfere with the fluorogen-interaction and partially or totally inhibit the activity of the functional domain. In this embodiment, the non-functional partner domain operates as a blocking domain. As illustrated in FIG. 5 for a protease cleavage embodiment, if the peptide linker is cleaved (or otherwise modified resulting in a conformational change in the peptide construct), then the non-functional blocking domain and the functional domain may at least partially dissociate. The at least partial dissociation may at least partially unblock the active portion of the functional domain, which may allow the functional domain to interact with a fluorogen and modulate its fluorescence signal.

The selection of a non-functional blocking domain to pair with a functional domain to form a synthetic hybrid scFv may be conducted using known genetic engineering techniques. Synthetic two-domain scFv genes may be constructed, for example, by digesting with appropriate restriction enzymes the full-length, two-domain plasmids coding for the scFvs selected from a yeast library as described above. The DNA coding for the variable domain of the scFv that does not contribute to the fluorogen interaction activity of the scFv may be removed from the digested plasmids. The removed DNA may be replaced with a new variable domain segment from a different scFv to form a hybrid plasmid. The fusion protein expressed from the hybrid plasmid may comprise a peptide construct comprising a synthetic hybrid scFv.

A hybrid scFv expressed as a surface protein (in a yeast surface-display system for example) from a hybrid plasmid as described above may be assayed for fluorogen interaction by flow cytometry, for example. In this manner, the fluorogen-interaction activity of a two-domain synthetic hybrid scFv may be determined, and non-functional blocking domains may be selected that inhibit (or completely block) the fluorogen-interaction activity of a functional domain.

The construction using molecular cloning methods of an artificial peptide construct comprising a pairing of unrelated V_{H} and V_{L} domains (one of which possesses fluorogen-interaction activity and one of which does not) covalently linked through a peptide linker, may serve as a platform for various embodiments described herein. Not wishing to be bound by theory, the natural association of some V_{H} and V_{L} domains and the interaction of their complementarity determining region (“CDR”) loops in the two-domain scFv architecture may somehow interfere with fluorogen interaction by the functional single domain in a hybrid scFv. By way of example, the interface between associated V_{H} and V_{L} domains may wholly or partially block the fluorogen-interacting active domain in the single functional V_{H} or V_{L} domain. Alternatively, or in addition, the association of the V_{H} and V_{L} domains may result in a rearrangement of the three-dimensional structure of the CDR loops of the active domain in the single functional V_{H} or V_{L} domain, which may inhibit interaction with a cognate fluorogen.

In various embodiments, the disclosed biosensors may comprise a peptide construct comprising a fluorogen-activating peptide comprising a functional fluorogen-interacting V_{H} or V_{L} domain, and a blocking peptide comprising a non-functional blocking domain of the opposite type. In various embodiments, the fluorogen-activating peptide comprising a V_{H} or V_{L} domain, and the blocking peptide comprising a variable domain of the opposite type may be linked through a peptide linker comprising an amino acid sequence that is specifically recognized as a cleavage substrate by a cognate protease, as a phosphorylation substrate by a cognate kinase, or as an acetylation substrate by a cognate acetyltransferase. In various embodiments, the biosensors may further comprise a phospho(amino acid) binding peptide or an acetyl(amino acid) binding peptide.

In various embodiments, when the peptide linker is modified (e.g., cleaved, phosphorylated, or acetylated), the peptide comprising a functional fluorogen-interacting V_{H} or V_{L} domain, and the peptide comprising a variable blocking domain of the opposite type, at least partially dissociate such that the peptide comprising a functional fluorogen-interacting V_{H} or V_{L} domain may interact with a fluorogen, thereby modulating the fluorescence signal produced by the fluorogen. In this manner, a peptide construct comprising a fluorogen-activating peptide linked to a blocking peptide through a peptide linker may function as a biosensor to detect and analyze enzyme (e.g., protease, kinase, acetyltransferase) activity.

Various embodiments disclosed herein will now be illustrated in the following, non-limiting examples.

### EXAMPLES

### Example 1

### scFvs that Specifically Interact with Fluorogen

scFvs that elicited fluorescence enhancement from three fluorogenic dyes (thiazole orange (“TO”), dimethyl indol red (“DIR”), and malachite green (“MG”)) were isolated. The scFvs were isolated using a yeast cell surface display library comprising approximately 10⁹ recombinant human scFvs derived from cDNA representing a naïve germline repertoire. The yeast cell surface display library was obtained from Pacific Northwest National Laboratory (PNNL). The materials, methods and protocols for using the PNNL yeast cell surface display library are described in the “Yeast Display scFv Antibody Library User's Manual,” Revision: MF031112, available from PNNL, Richland, Wash. 99352, USA (http://www.sysbio.org/dataresources/index.stm), the contents of which is incorporated by reference herein in its entirety. The methodology for the PNNL Yeast Display scFv Antibody Library was originally described in Feldhaus et al., Nat. Biotechnol., 21, 163-170, (2003).

The PNNL scFv library is specifically designed to display full-length scFvs whose expression on the yeast cell surface can be monitored with either N-terminal hemagglutinin (“HA”) or C-terminal c-myc epitope tags. These epitope tags allow monitoring by flow cytometry of scFv clones, or libraries of scFv clones, for surface expression of full-length scFv, for example. The extra cellular surface display of scFv by Saccharomyces cerevisiae also allows the detection of appropriately labeled antigen-antibody interactions by flow cytometry, for example. As a eukaryote, S. cerevisiae offers the advantage of post-translational modifications and processing of mammalian proteins, and therefore, is well suited for expression of human derived antibody fragments. In addition, the short doubling time of S. cerevisiae allows for the rapid analysis and isolation of antigen-specific scFv antibodies.

The PNNL yeast display system uses the a-agglutinin yeast adhesion receptor to display recombinant proteins on the surface of S. cerevisiae (Boder et al., Biotechnol. Prog., 14, 55, (1998); Boder et al., Nat. Biotechnol., 15, 553, (1997)). In S. cerevisiae, the a-agglutinin receptor acts as an adhesion molecule to stabilize cell-cell interactions and facilitate fusion between mating “a” and a haploid yeast cells. The receptor consists of two proteins, Aga1 and Aga2. Aga1 is secreted from the cell and becomes covalently attached to b-glucan in the extra cellular matrix of the yeast cell wall. Aga2 binds to Aga1 through two disulfide bonds, and after secretion remains attached to the cell via Aga1. The yeast display system takes advantage of the association of Aga1 and Aga2 proteins to display a recombinant scFv on the yeast cell surface.

The gene of interest is cloned into the pYD1 vector (Invitrogen), or a derivative of it, in frame with the AGA2 gene. The resulting construct is transformed into the EBY100 S. cerevisiae strain containing a chromosomal integrant of the AGA1 gene. Expression of both the Aga2 fusion protein from pYD1 and the Aga1 protein in the EBY100 host strain is regulated by the GAL1 promoter, a tightly regulated promoter that does not allow any detectable scFv expression in absence of galactose. Upon induction with galactose, the Aga1 protein and the Aga2 fusion protein associate within the secretory pathway, and the epitope-tagged scFv antibody is displayed on the cell surface. Molecular interactions with the scFv antibody can be easily assayed by incubating the cells with a ligand of interest. A combination of two rounds of selection using magnetic particles followed by two rounds of flow cytometric sorting will generally allow recovery of clones of interest.

The PNNL yeast display system may be utilized to isolate higher affinity clones from small mutagenic libraries generated from a unique antigen binding scFv clone (Boder et al., Proc. Natl. Acad. Sci. USA, 97, 10701, (2000)). Mutagenic libraries are constructed by amplifying the parental scFv gene to obtain higher affinity variants using error-prone PCR to incorporate 3 to 7 point mutations/scFv, for example. The material is cloned into the surface expression vector using the endogenous homologous recombination system present in yeast, known as “Gap-Repair”. Gap repair is an endogenous homologous recombination system in S. cerevisiae that allows gene insertion in chromosomes or plasmids at exact sites by utilizing as little as 30 base pair regions of homology between a gene of interest and its target site. This allows mutated libraries of clones to be rapidly generated and screened by selecting the brightest antigen binding fraction of the population using decreasing amounts of antigen relative to the K_{d} of the starting parental clone.

The PNNL yeast display system was utilized to clone scFvs that specifically bind the fluorogenic dyes thiazole orange (“TO”), malachite green (“MG”), dimethyl indol red (“DIR”), and derivatives thereof. EBY100 was host to the yeast display library and YVH10 was used to secrete scFvs as described in Feldhaus et al., Nat. Biotechnol., 21, 163-170, (2003). For analysis of individual scFvs, pPNL6 plasmids were transferred to JAR200 (Mat a ura3-52, trp1, leu2δ200, his3δ200, pep4:HIS3, prbd1.6R, can1, GAL, GAL promoter-AGA1::URA3:G418R). A modified PBS buffer (PBS pH 7.4, 2 mM EDTA, 0.1% w/v Pluronic F-127 (Molecular Probes, Invitrogen)) was used for magnetic bead enrichment, fluorescence-activated cell sorting (“FACS”) experiments, and all assays of yeast surface displayed or purified scFvs.

As used herein, the names of isolated and characterized scFvs consist of three components: i) the scFv chain configuration, with H designating the heavy variable (V_{H}) region and L designating the light variable (V_{L}) region; ii) a unique numerical identifier designating the parent isolate and its affinity maturation lineage in the format “parent#.1stgeneration#.2ndgenerationt” and iii) the fluorogenic dye used to isolate the scFv. Thus, for example, “HL1-TO1” indicates the parent isolate of the TO-activating, V_{H} and V_{L} clone 1, and “L5.1-MG” indicates the first affinity matured variant of the MG-activating, V_{L}-only clone 5.

The results are reported and discussed in Szent-Gyorgyi et al., Nat. Biotechnol., 26, 235-240, 2008; and in International Patent Application No. PCT/US2008/051962, each of which is incorporated by reference herein in its entirety. The DNA and amino acid sequences of the scFv fragments are disclosed in PCT/US2008/051962 and reproduced herein as SEQ ID NOS:21-40. These sequences are incorporated by reference herein in their entirety as though expressly listed herein.

### Example 2

### Genetic Dissection of Two-Domain scFvs

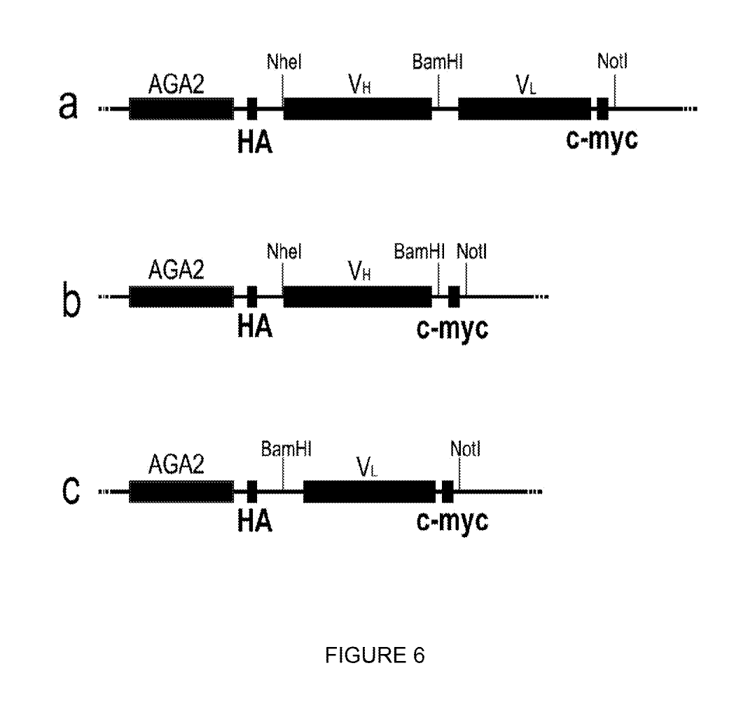

scFv genes in the Pacific Northwest National Laboratory yeast surface display library were cloned in a pPNL6 plasmid, where they were expressed as fusion proteins between an N-terminal HA-tagged AGA2p protein and a C-terminal c-myc epitope as shown in FIG. 6a. The V_{H} and V_{L} gene segments were linked by a flexible 15 amino acid peptide linker comprising 3 repeats of the sequence Gly₄Ser. Two-domain (V_{H} and V_{L}) scFv clones which were found to activate fluorescence in TO, MG and DIR fluorogens were reduced to their V_{H}-only and/or V_{L}-only plasmids by DNA manipulation.

Single variable domain reduction plasmids were constructed using restriction sites within the pPNL6 vector and the (Gly₄Ser)₃ peptide linker. V_{H} only plasmids were constructed by subcloning the V_{H} domain-coding restriction fragments into an empty pPNL6 vector. After cleavage with NheI and BamH1 restriction enzymes, V_{H} domain-coding fragments from 2-domain scFvs were separated from the rest of the plasmid DNA by agarose gel electrophoresis and purified with a QIAGEN Gel Extraction Kit (Qiagen Inc., Valencia, Calif. 91355, USA). A partial BamH1 restriction enzyme digest of an HL-A8-DIR 2-domain fluorogen-activating scFv was performed due to an internal BamHI restriction enzyme site in the A8-DIR V_{H} domain. Empty pPNL6 vector was cut using the same pair of restriction enzymes to remove the NheI/BamH1 stuffer fragment and the backbone was purified in a similar manner to prepare for ligation. V_{H} domains were then ligated into the pPNL6 vector backbone following the suggested protocol in an NEB Quick Ligation™ Kit (New England Biolabs Inc., Ipswich, Mass. 01938, USA).

A special vector pPNL6(HL1-TO1 V_{L}) was constructed in which to clone V_{L} domains. pPNL6 carrying the scFv gene HL1-TO1 was digested with BmtI (an isoschizomer of NheI) and BamH1. The DNA ends were treated with T4 DNA polymerase (according to the NEB protocol for blunting DNA ends, NEB Quick Blunting™ Kit). DNA was purified from this reaction using the QIAGEN PCR Cleanup Kit (Qiagen Inc., Valencia, Calif. 91355, USA). DNA molecules were circularized by ligation at a total DNA concentration <1 μg/ml. This series of enzymatic treatments deleted the HL1-TO1 V_{H} domain-coding DNA while retaining the V_{L} domain-coding DNA. It also restored the BamH1 site and preserved the reading frame between the Aga2 gene and the remaining V_{L} domain. V_{L} only plasmids were constructed by gel purifying the V_{L} domain-coding restriction fragments from all other two-domain plasmids after cleavage with BamH1 and NotI. The pPNL6(HL1-TO1 V_{L}) vector was cut using the same pair of enzymes to remove the HL1-TO1 V_{L} stuffer fragment and the backbone gel purified to prepare for ligation. V_{L} domains were ligated into this vector backbone following the suggested protocol in the NEB Quick Ligation™ Kit.

The modified single-domain (V_{H} or V_{L}) scFv genes were expressed from pPNL6 plasmids that generated surface displayed fusion proteins tagged with both HA and c-myc epitopes (FIGS. 6b and 6c).

### Example 3

### Single Variable Domains of scFvs that Specifically Interact with Fluorogen

Flow cytometry was used to measure both the amount of scFv (c-myc epitope) expressed, and the amount of fluorogen activating activity of the modified, surface-expressed scFvs. For each induced and un-induced sample, 10⁶ cells were washed twice in wash buffer (1× phosphate buffered saline, 2 mM EDTA, 0.1% Pluronic F-127) and re-suspended in 100 μl wash buffer containing 2 μg mouse monoclonal anti-c-myc antibody (Roche clone 9E10). Following a 1-hour incubation on ice, the cells are washed twice in wash buffer and re-suspended in 100 μl wash buffer containing 0.8 μg appropriately labeled goat anti-mouse secondary antibody. Alexa-fluor 647 conjugated secondary antibodies were used for TO dye activating scFvs. Alexa-fluor 488 conjugated secondary antibodies were used for MG and DIR activating scFvs. Alexa-fluor antibodies are available from Invitrogen.

The cells were again washed twice and re-suspended in 500 μl of wash buffer to which fluorogen was added to a concentration 10× the measured cell-surface K_{d} for the particular scFv. A parallel set of samples was treated with a 1 μM final concentration of propidium iodide, a vital dye used as a marker for cell viability. scFv activation of the fluorogen was assessed by flow-cytometry on a Becton Dickinson FACS Vantage SE cytometer. Fluorescence was excited using the 488 nm laser for TO fluorogen and the Alexa-fluor 488 conjugated antibodies and fluorescence signals were measured at 530 nm. MG and DIR fluorogen and Alexa-fluor 647 conjugated antibodies were excited using the 635 nm laser and fluorescence signals were measured at 685 nm. A ratio of fluorescence signal from the appropriate fluorogen channel and the c-myc channel was calculated to determine signal per scFv molecule in order to allow comparison of one scFv expressing cell sample to another.

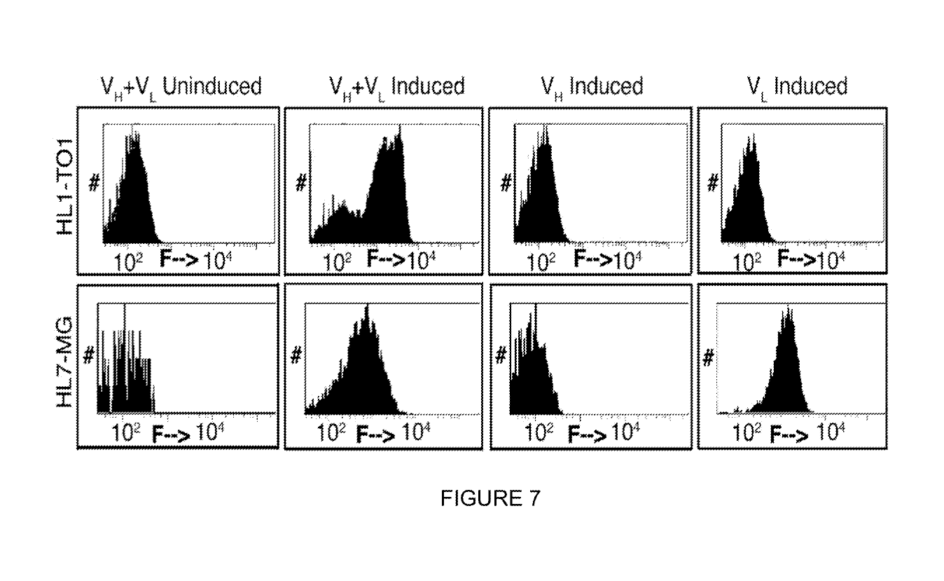

Analysis of the two-domain and single-domain peptides expressed from the engineered plasmids described in Example 2 provided results that fell into two distinct groupings, illustrated by the cytometric analyses shown in FIG. 7 (“FL” refers to full length scFvs, “HO” refers to variable heavy domain only single-domain scFv fragments, and “LO” refers to variable light domain only single-domain scFv fragments). For one group, illustrated, for example, by the scFv HL1-TO1, the full-length two-domain (FL) scFv shows typical fluorescence activation of the fluorogen compared to un-induced cells (significant numbers of cells appearing at higher fluorescence). However, neither of the individual V_{H} or V_{L} domains (V_{H}-only (HO) and V_{L}-only (LO), respectively) activated the fluorogen (no difference compared to un-induced cells). The other group, illustrated, for example, by HL7-MG, also shows typical fluorescence activation of the fluorogen by the full-length two-domain scFv compared to un-induced cells. However, the molecular dissection of the V_{H} and V_{L} domains reveals that the fluorogen-activating activity resides completely in the V_{L} domain.

### Example 4

### Quantification of Fluorogen Activation by Single-Domains of scFvs

The amount of scFv expressed on the surface of the yeast cells described in Example 3 was determined by fluorescence labeling of the c-myc epitope fused to the C-terminal end of each scFv. The population average fluorescent intensity of the c-myc signal was used to normalize the population average fluorogen activation signal to provide a quantification of the fluorogen activation by the surface-displayed scFvs. These results are presented in Table 1, where the fluorogenic activity of each dissected construct (i.e., the isolated single-domain scFvs) is expressed as a percentage of the fluorogenic activity of its parent two-domain clone (ΔH and ΔL refer to the removed domain in each dissected construct).

TABLE 1
Fluorogen activating activity of scFvs

| | Percent |
|---|---|
| | Fluorogenic |
| scFv | Activity |
| HL1-TO1 | 100.0 |
| H(ΔL)1-TO1 | 9.6 |
| (ΔH)L1-TO1 | 6.4 |
| HL4-MG | 100.0 |
| H(ΔL)4-MG | 1.7 |
| (ΔH)L4-MG | 1.7 |
| HL7-MG | 100.0 |
| H(ΔL)7-MG | 3.7 |
| (ΔH)L7-MG | 114.8 |
| HL9-MG | 100.0 |
| H(ΔL)9-MG | 7.1 |
| (ΔH)L9-MG | 111.9 |
| HL-A8-DIR | 100.0 |
| H(ΔL)-A8-DIR | 4.2 |
| (ΔH)L-A8-DIR | 3.9 |
| HL-J6-DIR | 100 |
| H(ΔL)-J6-DIR | 3.2 |
| (ΔH)L-J6-DIR | 3.1 |
| HL-K7-DIR | 100.0 |
| H(ΔL)-K7-DIR | 3.2 |
| (ΔH)L-K7-DIR | 4.2 |
| HL-K10-DIR | 100.0 |
| H(ΔL)-K10-DIR | 16.8 |
| (ΔH)L-K10-DIR | 88.6 |
| HL-M8-DIR | 100.0 |
| H(ΔL)-M8-DIR | 18.9 |
| (ΔH)L-M8-DIR | 72.0 |

The quantitative data presented in Table 1 indicate that five of the scFvs require both V_{H} and V_{L} domains for fluorogen activation activity. That is, individual V_{H} or V_{L} domains retain only a few percent of the fluorogen activation activity of the parent scFv. The data also indicate that four scFvs possessed fluorogen activation activity that can be attributed to a single variable domain. Expression of the V_{L} domain of HL7-MG, HL9-MG, HL-K10-DIR, or HL-M8-DIR is sufficient to activate the fluorescence of the cognate fluorogen for each scFv. Sequence analysis of the individual domains of HL7-MG and HL9-MG reveals 92% sequence identity at the protein level for the V_{L} domains, while the V_{H} domains share approximately 75% sequence identity. The V_{L} domains of HL-K10-DIR and HL-M8-DIR are 100% identical while the V_{H} domains share approximately 46% sequence identity.

### Example 5

### Construction of Synthetic Hybrid Two-Domain scFvs

Synthetic two-domain hybrid scFvs comprising various single active variable domains and single inactive variable domains of the opposite type were constructed and the activity of the hybrids was measured. For the single active variable domains in certain hybrids, a V_{H} domain (H6-MG) was used. For the single active variable domains in certain other hybrids, V_{L} domains from HL7-MG, HL9-MG, L5.1-MG, and HL-M8-DIR were used.

The synthetic two-domain scFv genes were constructed by DNA manipulation of the corresponding gene segments in the yeast surface-display vector pPNL6. Full-length, two-domain plasmids were digested with appropriate restriction enzyme to remove the variable domain-coding DNA of interest, which was then physically replaced with a new variable domain segment. All scFv genes were in pPNL6 vector backbones. Thus for swapping V_{H} domains NheI/BamHI restriction digests were performed to remove and replace V_{H} domains. BamHI/NotI restriction digests were performed to remove and replace V_{L} domains (FIG. 6a). Plasmid backbones and domain fragments were purified by gel electrophoresis. Purified V_{H} and V_{L} domains and plasmid backbones from different starting plasmids were combined and joined by DNA ligation to form new full-length hybrid genes.

Plasmid DNA in ligation reactions were transformed into chemically competent TOP10 or Mach1™ E. coli (Invitrogen) or electroporation competent DH5α E. coli (Bioline). Plasmid DNA was extracted from E. coli using a miniprep kit (QIAGEN). scFv genes in all plasmids were re-sequenced to confirm they contained the correct fragments and were in frame with the c-myc epitope (GeneWiz).

Selective growth media (SD+CAA) and induction media (SGR+CAA) for yeast carrying the pPNL6 surface display plasmid have been previously described (see, e.g., Feldhaus et al., Nat. Biotechnol., 21, 163-170, 2003; and Yeast Display scFv Antibody Library User's Manual,” Revision: MF031112, available from PNNL, Richland, Wash. 99352, USA). pPNL6 plasmids containing the scFv hybrid gene constructs were transformed into EBY100 yeast by EZ Yeast Transformation Kit (BIO 101, Vista, Calif. 92083, USA). The EBY100 yeast transformants were grown for 48 hours at 30° C. in SD+CAA selective growth media. When the cultures reached an optical density at 660 nm of >1.0 (>2×10⁷ cells/ml) the yeast cells were harvested by centrifugation and re-suspended in SGR+CAA selective induction medium at a concentration of 2×10⁷ cells/ml. These induction cultures were incubated with shaking for 72 hours at 20° C. For each transformant, an additional culture was maintained in selective growth media as an un-induced control.

### Example 6

### Quantification of Fluorogen Activation by Hybrid Two-Domain scFvs

Fusion proteins expressed on the surface of yeast were assayed for fluorogen activation by flow cytometry and normalized, as above, by determining the total amount of surface displayed c-myc-tagged fusion protein. The activity of the parent single active variable domain was set to 100% and the hybrid scFv activity expressed relative to the parent. The results are presented in Table 2.

TABLE 2
Fluorogen activating activity of hybrid scFvs

| | | Percent |
|---|---|---|
| | | Fluorogenic |
| V_{H} domain | V_{L} domain | Activity |
| H6-MG | — | 100 |
| H6-MG | HL1-TO1 | 0.7 |
| H6-MG | HL4-MG | 79.5 |
| — | HL7-MG | 100 |
| HL4-MG | HL7-MG | 4.3 |
| HL9-MG | HL7-MG | 45.7 |
| — | HL9-MG | 100 |
| HL4-MG | HL9-MG | 5.4 |
| HL7-MG | HL9-MG | 95.6 |
| — | L5.1-MG | 100 |
| HL4-MG | L5.1-MG | 38.3 |
| HL1-TO1 | L5.1-MG | 130.1 |
| HL7-MG | L5.1-MG | 125.2 |
| HL9-MG | L5.1-MG | 87.8 |
| — | HL-M8-DIR | 100 |
| HL1-TO1 | HL-M8-DIR | 23.4 |
| HL4-MG | HL-M8-DIR | 3.1 |

As shown in Table 2, the fluorogen activating activity of some of the active single-domains is inhibited (or blocked) by the presence of various partner domains. For example, the fluorogen-activating activity of the V_{H} domain H6-MG is blocked greater than 99% by the presence of the V_{L} domain of HL1-TO1, and the activity of the V_{L} domains of HL7-MG, HL9-MG and HL-M8-DIR is blocked approximately 95% by the V_{H} domain of HL4-MG.

### Example 7

### Isolation and Purification of One-Domain scFvs and Two-Domain Hybrid scFvs

In order to verify that the activity modulation of the single-domain scFvs was not due to an artifact of the yeast surface display of the protein, soluble versions of the fluorogenically active V_{H} single-domain of H6-MG and the “blocked construct” hybrid scFv comprising the V_{H} domain of H6-MG and the V_{L} domain of HL1-TO1 were expressed in E. coli, isolated and purified.

The genes encoding for single-domain scFvs and hybrid blocked two-domain scFvs were isolated from pPNL6 clones and tailed with SfiI restriction enzyme sites by anchored PCR. The forward primer for amplifying and SfiI-tailing the H6-MG gene is:

5′-GGCCCAGCCGGCCATGGCGCAGGTGCAGCTGCAGGAGTGC-3′.

The reverse primer for amplifying and SfiI-tailing the H6-MG gene is:

5′-GGCCCCCGAGGCCTCGGAGACAGTGACCAGGGTACC-3′.

The forward primer for amplifying and SfiI-tailing the two-domain hybrid containing the V_{H} domain of H6-MG and the V_{L} domain of HL1-TO1 is the same. The reverse primer for amplifying and SfiI-tailing the two-domain hybrid containing the V_{H} domain of H6-MG and the V_{L} domain of HL1-TO1 is:

5′-GGCCCCCGAGGCCCCTAGGACGGTGAGCTTGGTCC-3′.

PCR products were TOPO cloned (Invitrogen) and sequenced to verify faithful amplification. SfiI fragments were gel purified after SfiI digestion and ligated into SfiI-digested pAK400 (Krebber et al., J. Immuno. Meth., 201, 35-55, 1997) between a pelB leader sequence and His₆ tag. pAK400 is an E. coli periplasmic secretion vector for high-level expression of scFvs under the control of a wild-type lac promoter and IPTG inducible promoter. E. coli transformed with the pAK400 plasmids were grown to late log phase and induced with 1 mM IPTG in fresh media for 5 hours at 25° C.

Periplasmic proteins were isolated by osmotic shock (Maynard et al., J. Immuno. Meth., 306, 51-67, 2005) and dialyzed in a 10 mM Tris, 500 mM NaCl buffer, pH 8.0. Periplasmic, secreted scFvs were purified via the C-terminal His₆-tag by Nickel-NTA chromatography (QIAGEN). The periplasmic protein extract from 1 liter of culture was incubated with 0.5 ml settled volume of Nickel-NTA resin for 1 hour. The column was poured with this resin and the flow-through applied to the column a second time. The column was washed with buffer containing 20 mM imidazole, 10 mM Tris, 100 mM sodium phosphate, 300 mM NaCl, pH 8.0. His₆-tagged protein was eluted in buffer containing 250 mM imidazole, 10 mM Tris, 100 mM sodium phosphate, 300 mM NaCl, pH 8.0, and collected in 8, 500 μL fractions. All purification steps and storage of proteins were performed at 4° C. All fractions along with initial flow through and washes were analyzed by 15% SDS-PAGE gel electrophoresis to monitor purification.

This procedure was used to successfully produce isolated homogenous and soluble versions of the fluorogenically active V_{H} single-domain of H6-MG and the “blocked construct” hybrid comprising the V_{H} domain of H6-MG and the V_{L} domain of HL1-TO1.

### Example 8

### Isolated One-Domain scFv and Two-Domain Hybrid scFv Activity Assay

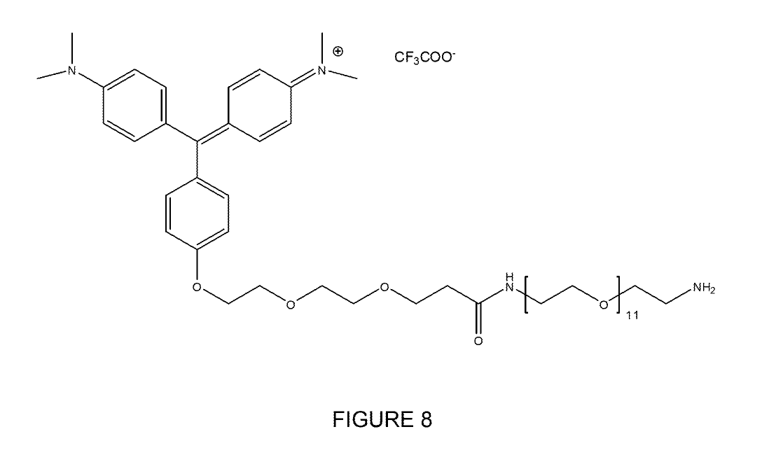

Fluorogen-titration experiments were performed with the soluble proteins isolated in Example 7 to determine the K_{d} of MG for each protein. The fluorogen titration analyses were performed on a TECAN Saffire2 plate reader in black, 96-well, flat bottom microtiter plates. 500 ng of purified protein was mixed with MG-11P-NH₂ fluorogen (FIG. 8) in wash buffer in a final volume of 100 μl. Fluorogen concentrations varied from 0 to 20 μM in a 3-fold serial dilution series. MG fluorogen samples were excited at 625 nm and emission was detected at 660 nm.

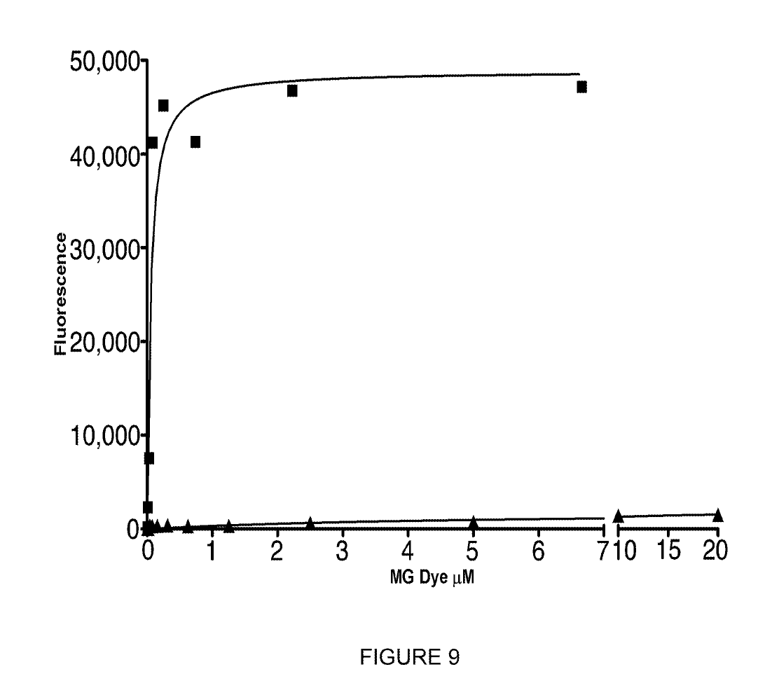

The results of the titrations are shown in FIG. 9. The K_{d} of H6-MG was measured in these experiments to be 50 nM. The apparent K_{d} of the blocked construct was at least 4.9 μM. The solution K_{d} of the blocked construct is at least two orders of magnitude higher than the active single-domain that is contained in the hybrid scFv. These results confirm that it is the partnering of the two domains in the hybrid that inhibits the activity of the H6-MG V_{H} domain and not some artifact of the protein's location on the yeast cell surface.

### Example 9

### Comparison of the Fluorogenic Activity for a One-Domain scFv and a Two-Domain Hybrid scFv

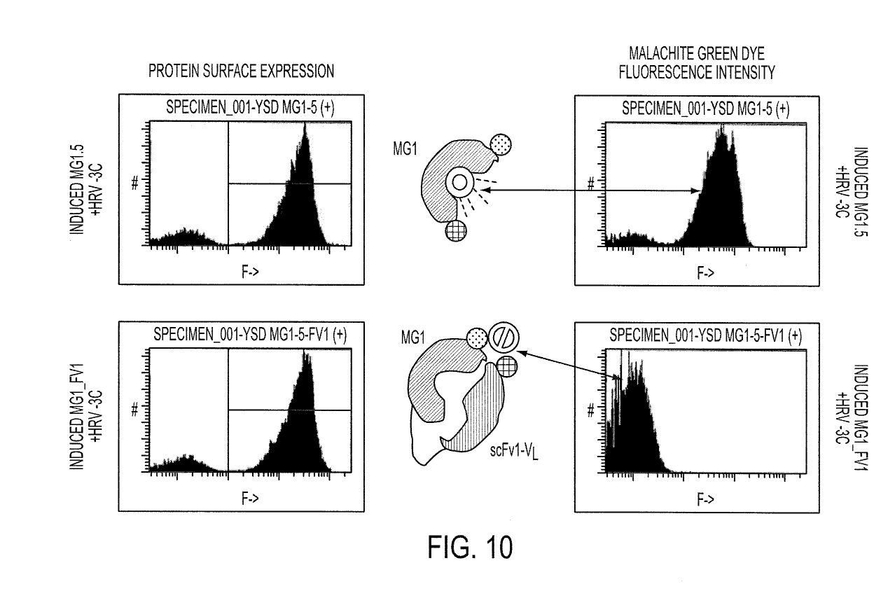

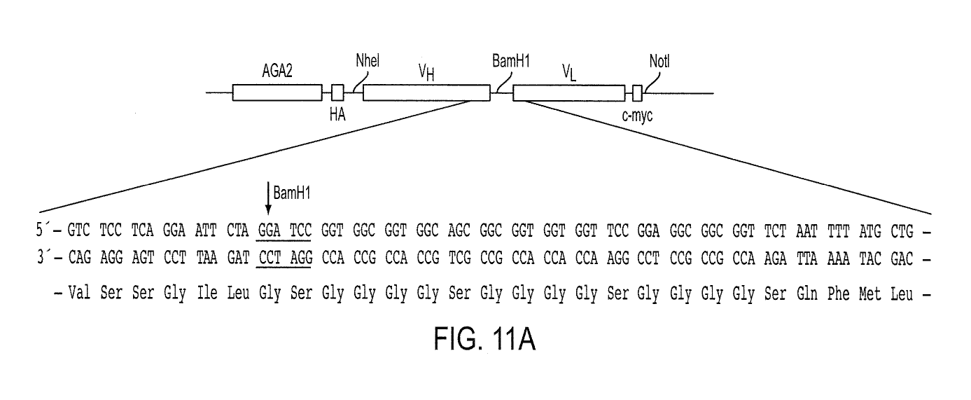

As described above, the MG fluorogen-activating protein H6-MG comprises a single V_{H} domain, and the TO fluorogen-activating protein HL1-TO1 comprises a two-domain (V_{H} and V_{L}) structure. The DNA for the V_{H} domain of H6-MG was combined in vitro with the DNA for the V_{L} domain of HL1-TO1 to form a fusion peptide construct of the two unrelated domains. This construct DNA was introduced into yeast and the protein product produced on the surface of the yeast as described above. The surface-expressed hybrid scFv construct was assayed for the ability to bind and activate MG fluorogen by FACS. The results are presented in FIG. 10, wherein “MG1” indicates the single V_{H} domain of H6-MG, “scFv1” indicates the V_{L} domain of HL1-TO1, and “HRV-3C” indicates human rhinovirus 3C protease.

The left hand column of FACS data in FIG. 10 indicate that both the single V_{H} domain of H6-MG and the hybrid H6-MG (V_{H})/HL1-TO1(V_{L}) construct are well expressed on the surface of yeast as determined by number of counts (area of the peak) in the P5 window, which correlates with the amount of c-myc epitope expressed on the surface of the yeast cells. The amount of MG fluorogen activation by the two different scFvs is shown by the counts between the 10³ and 2×10⁴ units on the X-axis of the right-hand column of FACS data. The data indicate that while the single domain H6-MG scFv activates the MG fluorogen, the fluorogen is not activated by the hybrid H6-MG (V_{H})/HL1-TO1(V_{L}) construct (>99% inhibition). Accordingly, the hybrid H6-MG (V_{H})/HL1-TO1(V_{L}) construct is effectively blocked. The respective scFvs are illustrated by the diagrams presented between the two columns of FACS data (the diagrams match the respective plots of FACS data).

### Example 10

### Construction of a Blocked scFv Having an HRV-3C Protease Substrate

The DNA sequence coding for the peptide linker that covalently links the V_{H} and V_{L} domains in the blocked scFvs described in Example 9 was manipulated to add a DNA sequence (SEQ ID NO:10) that would code for the peptide sequence Leu-Glu-Val-Leu-Phe-Gln-Gly-Pro (SEQ ID NO:9). This peptide sequence is recognized and cleaved by the Human Rhinovirus 3C (“HRV-3C”) protease between the Gln and Gly residues.

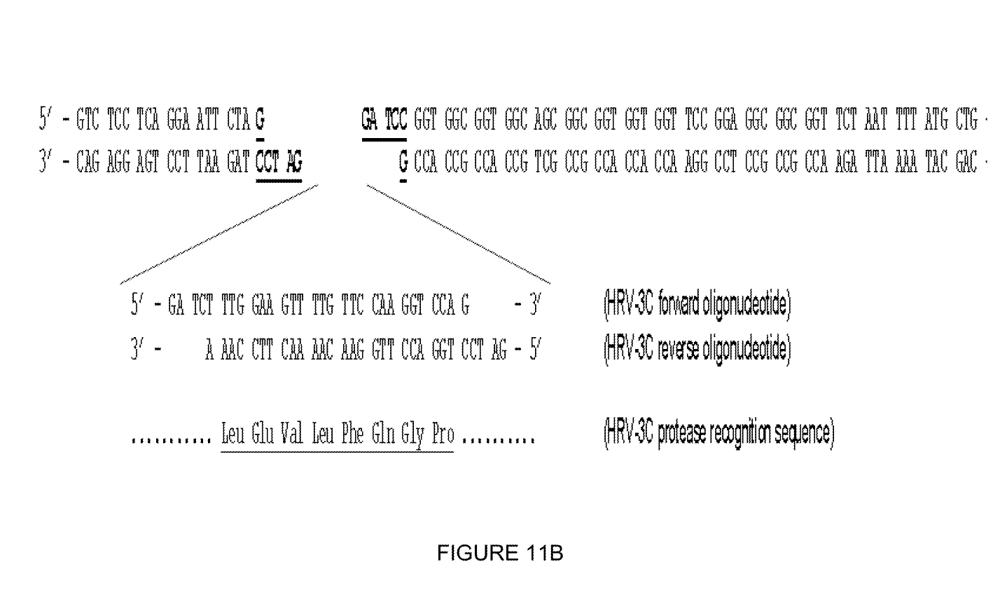

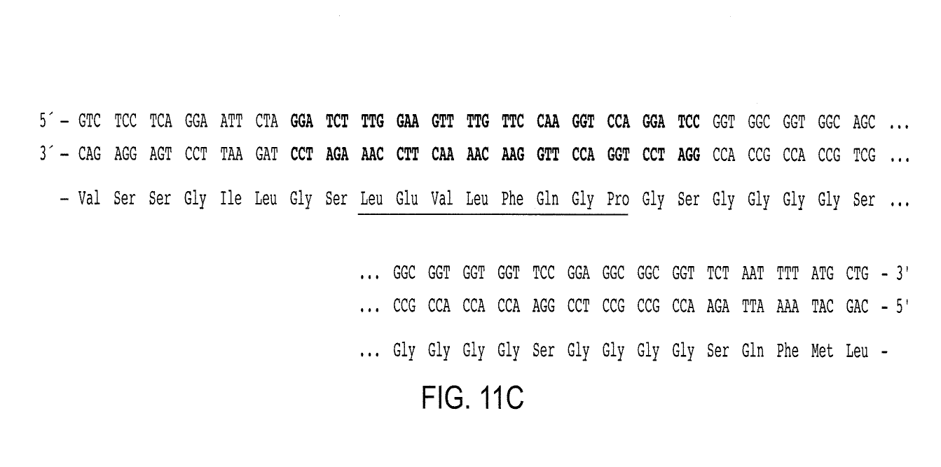

The gene segment shown in FIG. 11A is part of the plasmid pPNL6. The plasmid pPNL6 was used to construct blocked scFvs as described above. The DNA encoding for the peptide linker segment connecting the V_{H} and V_{L} domains in the blocked scFvs comprised a recognition sequence that was cleaved by BamH1 restriction enzyme. In this example, the DNA comprised a 5′-GGA TCC- sequence and a 3′-CCT AGG- sequence, which was cleaved by BamH1 restriction enzyme as indicated in FIG. 11B.

A DNA segment coding for the peptide sequence Leu-Glu-Val-Leu-Phe-Gln-Gly-Pro and comprising BamH1 compatible ends was joined to the BamH1-cleaved plasmid using standard DNA ligation techniques known in the art. The two complementary DNA fragments encoding for the peptide sequence Leu-Glu-Val-Leu-Phe-Gln-Gly-Pro (i.e., the HRV-3C recognition sequence forward and reverse oligonucleotides) were synthesized and used to form duplex DNA using standard annealing techniques. The double stranded oligonucleotide was ligated into the cleaved plasmid in the peptide linker region. The modified DNA sequence (and expressed peptide) is illustrated in FIG. 11C. The resulting plasmid comprised a DNA sequence (SEQ ID NO:48) comprising an HRV-3C protease recognition sequence, which when expressed, resulted in a peptide construct (SEQ ID NO:47) comprising a fluorogen-activating peptide and a blocking peptide linked to the fluorogen-activating peptide through a peptide linker comprising an amino acid sequence that was specifically recognized as a modification substrate by a cognate enzyme, in this example HRV-3C protease.

### Example 11

### Fluorogenic Activity of a Blocked scFv Having a Protease Substrate

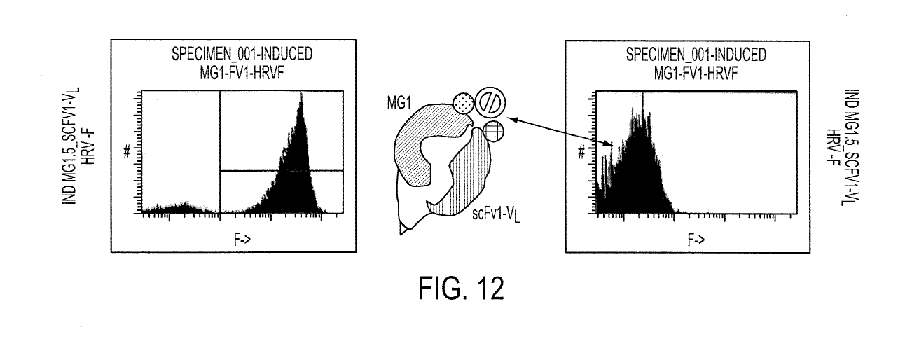

The scFv gene described in Example 10 (comprising SEQ ID NO:48) was introduced into yeast and the protein product expressed on the surface of yeast using the methods described above. The surface-displayed peptide constructs were assayed using FACS for the ability of the constructs to interact with and activate MG fluorogen. The results of the FACS analyses are shown in FIG. 12 (the plot on the left hand side corresponds to c-myc and the plot on the right hand side corresponds to fluorogen). A data analysis similar to that described in connection with FIG. 10, above, indicated that the scFv comprising the modified linker was still well expressed on the surface of the yeast (area of the peak in the P5 window in the left panel) and still fails to show fluorogenic activation of MG (lack of a peak between 10³ and 2×10⁴ units on the X-axis of the plot in the right panel). The diagram between the two plots depicts the same domains as in FIG. 10 with the addition of the HRV-3C protease cleavage substrate.

### Example 12

### Fluorogenic Activity of a Blocked scFv Having a Protease Substrate and Treated with Protease

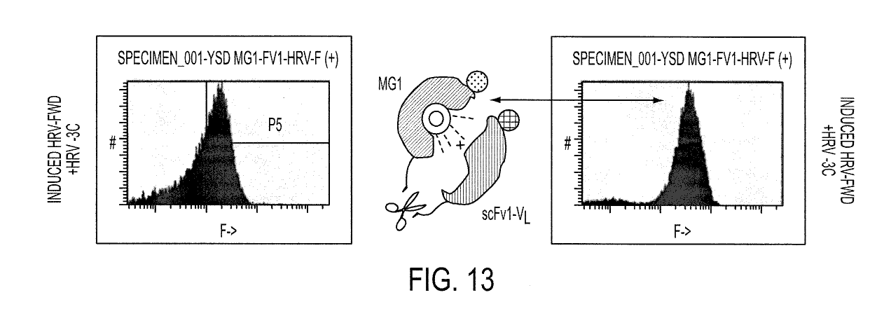

Yeast cells comprising surface-displayed scFv comprising the modified linker described in Example 11 were assayed by FACS to determine surface expression of the peptide construct. The yeast cells were then treated with 1 unit of HRV-3C protease overnight at 4° C. and then re-assayed by FACS to quantify cleavage (indicated by the loss of c-myc epitope signal) and to quantify MG fluorescence activation. The results of the FACS analyses are shown in FIG. 13 (the plot on the left corresponds to c-myc and the plot on the right corresponds to fluorogen).

Comparing the data presented in FIG. 12 with the data presented in FIG. 13 indicates that the majority of the c-myc epitope was cleaved off of the surface of the yeast (reduced area of red peak in the P5 window in the left panel) concomitant with a large fluorogenic activation of MG (shift of the peak to between 10³ and 2×10⁴ units on the X-axis of the right-hand panel). The diagram between the two plots schematically depicts this data by showing the cleavage of the HRV-3C substrate, the dissociation of the two domains from each other, and the activation of the fluorogen molecule.

The peptide constructs produced in the above examples comprised a fluorogen-activating peptide (comprising H6-MG (V_{H})) and a blocking peptide (comprising HL1-TO1 (V_{L})) linked to the fluorogen-activating peptide through a peptide linker comprising an amino acid sequence that is specifically recognized as a cleavage substrate by an HRV-3C protease. The constructs find utility as biosensors for protease activity.

### Example 13

### Kinetic Analysis of Yeast Surface Displayed HRV-3C Biosensors

The potential for the biosensor described in Example 12 to be used as a real time assay for detecting the amount of protease in a sample was explored by evaluating the kinetics of fluorescence activation for the biosensor and other control constructs in a TECAN analytical fluorimeter. In these analyses, 10⁶ yeast cells expressing different scFv constructs on their cell surface were treated with 1 unit of HRV-3C protease at 25° C. All incubations were conducted in the presence of MG fluorogen. The ability of the constructs to activate MG fluorescence was measured over a period of 2 hours.

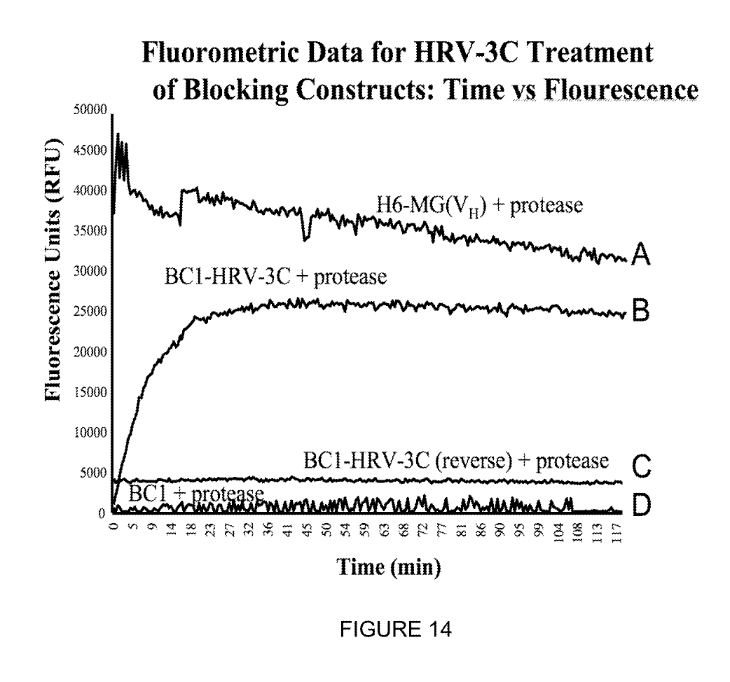

FIG. 14 shows the data for this kinetic assay of the activation of the HRV-3C protease biosensor. Line B is the activation curve for the biosensor described in Example 12. Activation of the biosensor is complete by 30 minutes of incubation. The fluorescence signal plateaus for the remainder of the assay time. The length of time to reach the plateau may be used as a measure of the protease concentration. Line C is the response of the same blocked construct when a different amino acid sequence (which is not a protease substrate) of the same length was inserted into the peptide linker. There was no change in activity upon treatment with HV-3C protease. Line D is the activation profile of the “blocked construct” without any added amino acid sequence in the peptide linker. There was a very small signal that did not change with time. Line A is the signal of the active single-domain H6-MG V_{H} domain expressed on the surface of yeast.

### Example 14

### Construction of a Blocked scFv Having a Caspase 3 Protease Substrate

The DNA sequence coding for the peptide linker that covalently links the V_{H} and V_{L} domains in the blocked scFvs described in Example 9 was manipulated to add a DNA sequence (SEQ ID NO:14) that would code for the peptide sequence Asp-Glu-Val-Asp (SEQ ID NO:13). This peptide sequence is recognized and cleaved by caspase 3 protease.

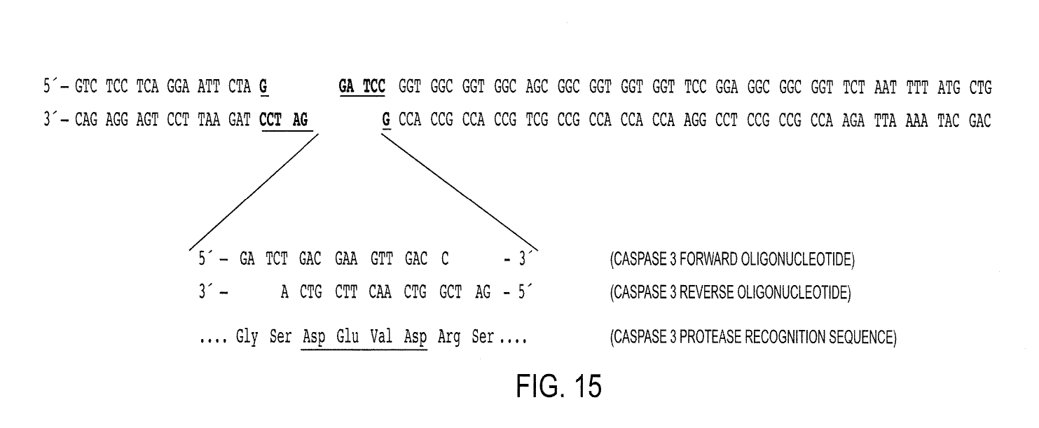

The plasmid pPNL6 was used to construct blocked scFvs as described above. The DNA encoding for the peptide linker segment connecting the V_{H} and V_{L} domains in the blocked scFvs comprised a recognition sequence that was cleaved by BamH1 restriction enzyme. In this example, the DNA comprised a 5′-GGA TCC- sequence and a 3′-CCT AGG- sequence, which was cleaved by BamH1 restriction enzyme as indicated in FIG. 15.

A DNA segment coding for the peptide sequence Asp-Glu-Val-Asp and comprising BamH1 compatible ends was joined to the BamH1-cleaved plasmid using standard DNA ligation techniques known in the art. The resulting plasmid comprised a DNA sequence (SEQ ID NO:52) comprising a caspase 3 protease recognition sequence, which when expressed, resulted in a peptide construct (SEQ ID NO:51) comprising a fluorogen-activating peptide and a blocking peptide linked to the fluorogen-activating peptide through a peptide linker comprising an amino acid sequence that is specifically recognized as a modification substrate by a cognate enzyme, in this example caspase 3 protease.

The peptide constructs produced in this example comprised a fluorogen-activating peptide (comprising H6-MG (V_{H})) and a blocking peptide (comprising HL1-TO1 (V_{L})) linked to the fluorogen-activating peptide through a peptide linker comprising an amino acid sequence that was specifically recognized as a cleavage substrate by caspase 3 protease. The constructs find utility as biosensors for protease activity.

### Example 15

### Kinetic Analysis of Yeast Surface Displayed Caspase 3 Biosensors

The potential for the biosensor described in Example 14 to be used as a real time assay for detecting the amount of protease in a sample was explored by evaluating the kinetics of fluorescence activation for the biosensor and other control constructs in a TECAN analytical fluorimeter. In these analyses, 10⁶ yeast cells expressing different scFv constructs on their cell surface were treated with 1 unit of caspase 3 protease at 25° C. All incubations were conducted in the presence of MG fluorogen. The ability of the constructs to activate MG fluorescence was measured over a period of 2 hours.

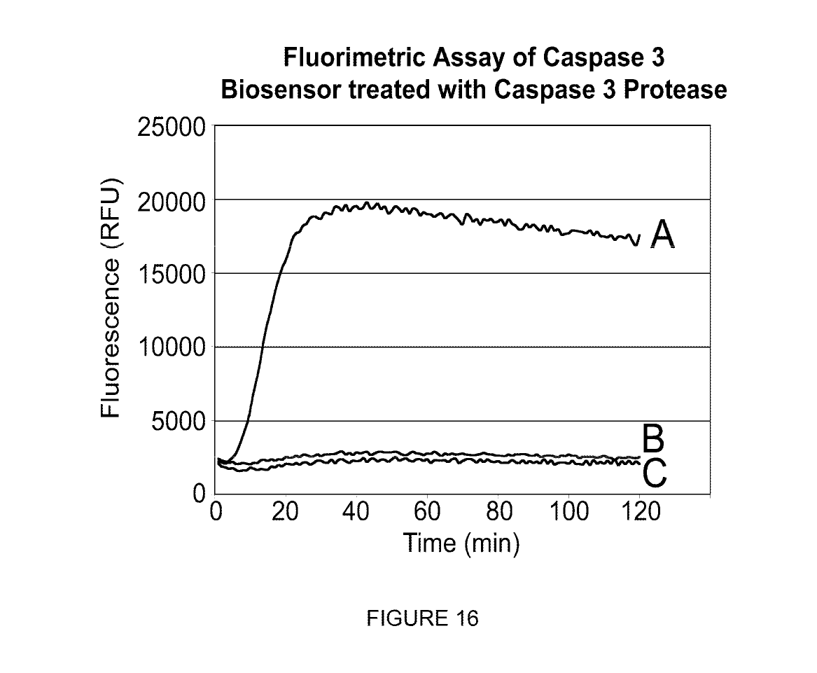

FIG. 16 shows the data for this kinetic assay of the activation of the caspase 3 protease biosensor. Line A is the activation curve for the biosensor described in Example 14. Activation of the biosensor is complete by 30 minutes of incubation. The fluorescence signal plateaus for the remainder of the assay time. The length of time to reach the plateau may be used as a measure of the protease concentration. Line B is the activation profile of the biosensor in the absence of caspase 3 protease. Line C is the activation profile of the “blocked construct” without any added amino acid sequence in the peptide linker.

### Example 16

### Isolation and Purification of Caspase 3 Biosensors

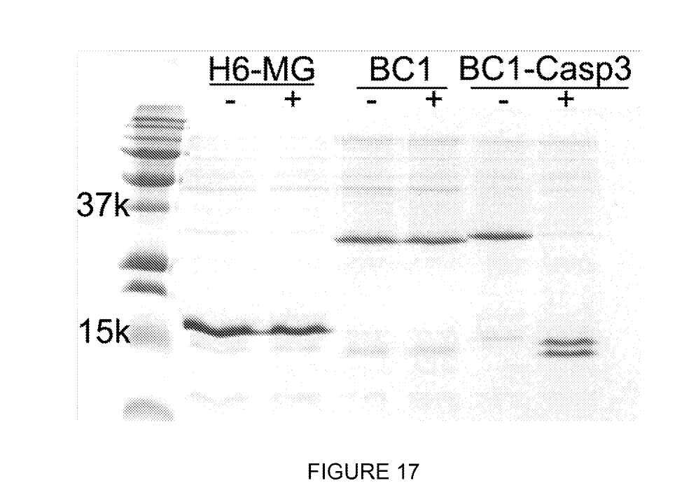

Caspase 3 biosensors as described in Example 14 were isolated and purified as described in Example 7. 1 μg of single-domain active V_{H}, 1 μg of blocked scFv without modified peptide linker, and 1 μg of purified biosensor were respectively mixed with 1 unit of caspase 3 or buffer solution and incubated overnight at 4° C. The proteins were run on an 18% SDS gel and stained with coomasie blue. A photograph of the gel is presented in FIG. 17 (“H6-MG” indicates single domain V_{H}, “BC1” indicates a blocked peptide construct without a recognition sequence inserted into the peptide linker, and “BC1-Casp3” indicates the active biosensor construct; (−) indicates incubation with buffer alone, and (+) indicates incubation with caspase 3 protease).

### Example 17

### In Vivo Stability and Functionality of Caspase 3 Biosensors

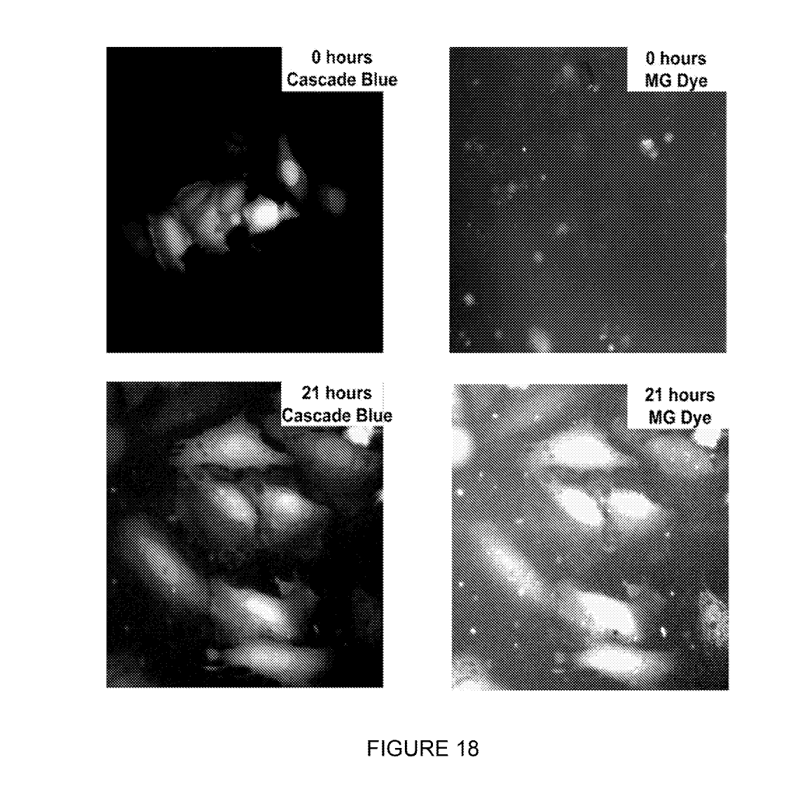

The in vivo stability and functionality of isolated and purified caspase 3 biosensors as described in Example 16 were evaluated. HeLa cells were injected with Cascade Blue dextran tracking solution comprising 12 mg/ml biosensor and 10 μg MG fluorogen. The cells were treated with 10 μg/ml etoposide. Microscopy images were acquired in both the blue and MG channels immediately after injection and at 21 hours post-injection. Representative microscopy images are presented in FIG. 18.

### Example 18

### Construction of an HRV-3C Protease Biosensor

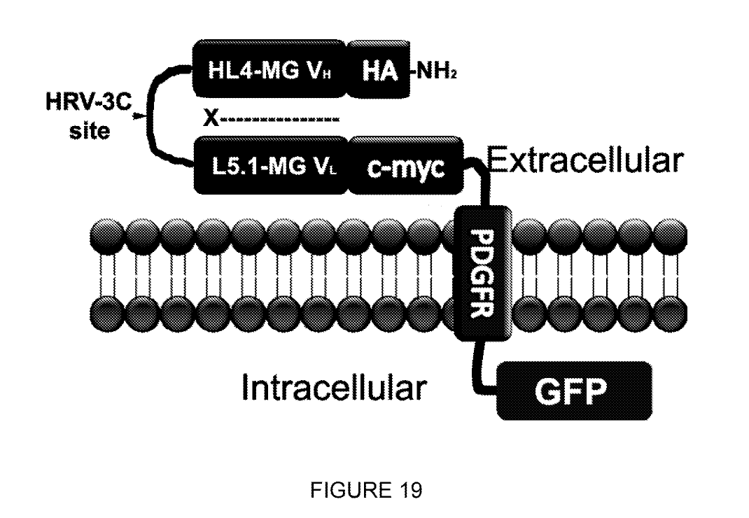

A membrane-bound biosensor was constructed in a pBabe-Sac-Lac retroviral vector using genetic engineering methods known in the art. The fluorogen-activating peptide of the biosensor comprised the L5.1-MG V_{L} domain and the blocking peptide of the biosensor comprised the HL4-MG V_{H} domain (Example 6). The peptide linker was modified as described in Example 10 to include an HRV-3C protease recognition site. NIH 3T3 cells were transduced with the retroviral vector expressing the HRV-3C biosensor using genetic engineering methods known in the art. The biosensor is illustrated in FIG. 19. The biosensor is positioned extracellularly, connected to green fluorescent protein (“GFP”) through a transmembrane PFGER peptide.

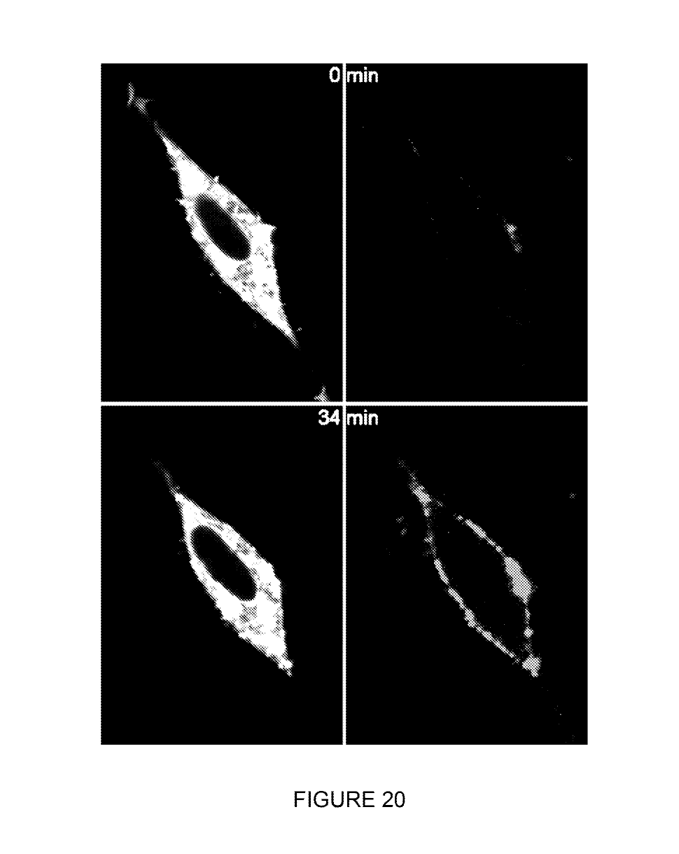

The NIH T3T cells expressing the biosensor-GFP fusion protein construct were treated with HRV-3C protease. Microscopy images were acquired in both the GFP channel and MG channel immediately after contact and after 34 minutes incubation. Representative microscopy images are presented in FIG. 20 (left panels in GFP channel, right panels in MG channel).

### Example 19

### Construction of a Caspase 1 Protease Biosensor

The DNA sequence coding for the peptide linker that covalently links the V_{H} and V_{L} domains in the blocked scFvs described in Example 9 was manipulated to add a DNA sequence (SEQ ID NO:12) that would code for the peptide sequence Tyr-Val-Ala-Asp (SEQ ID NO:11). This peptide sequence is recognized and cleaved by caspase 1 protease.

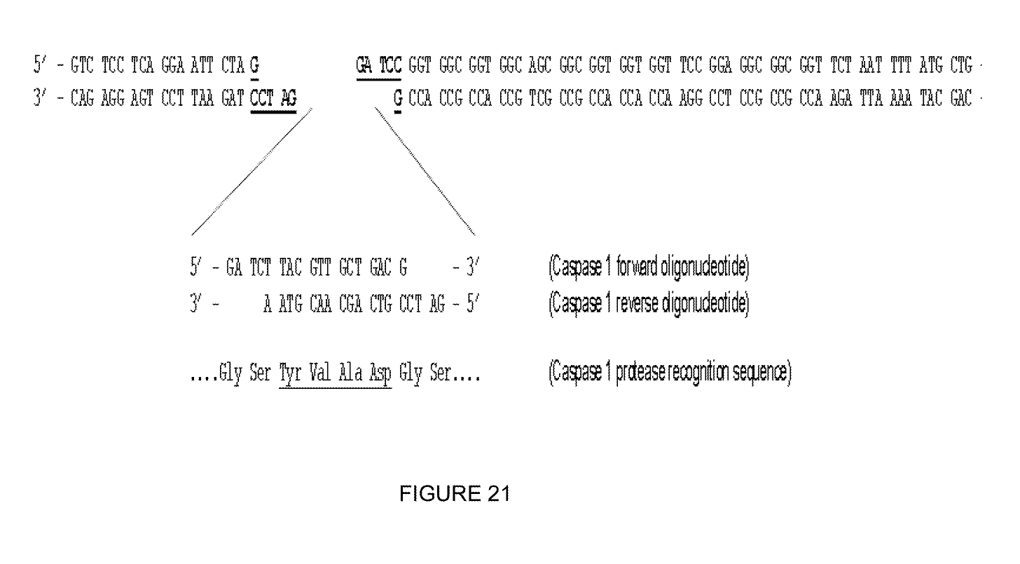

The plasmid pPNL6 was used to construct blocked scFvs as described above. The DNA encoding for the peptide linker segment connecting the V_{H} and V_{L} domains in the blocked scFvs comprised a recognition sequence that was cleaved by BamH1 restriction enzyme. In this example, the DNA comprised a 5′-GGA TCC- sequence and a 3′-CCT AGG- sequence, which was cleaved by BamH1 restriction enzyme as indicated in FIG. 21.

A DNA segment coding for the peptide sequence Tyr-Val-Ala-Asp and comprising BamH1 compatible ends was joined to the BamH1-cleaved plasmid using standard DNA ligation techniques known in the art. The resulting plasmid comprised a DNA sequence (SEQ ID NO:50) comprising a caspase 1 protease recognition sequence, which when expressed, resulted in a peptide construct (SEQ ID NO:49) comprising a fluorogen-activating peptide and a blocking peptide linked to the fluorogen-activating peptide through a peptide linker comprising an amino acid sequence that is specifically recognized as a modification substrate by a cognate enzyme, in this example caspase 1 protease.

The peptide constructs produced in this example comprise a fluorogen-activating peptide (comprising H6-MG (V_{H})) and a blocking peptide (comprising HL1-TO1 (V_{L})) linked to the fluorogen-activating peptide through a peptide linker comprising an amino acid sequence that is specifically recognized as a modification substrate by a protease. The constructs may find utility as biosensors for protease activity.

### Example 20

### Construction of a TEV Protease Biosensor

The DNA sequence coding for the peptide linker that covalently links the V_{H} and V_{L} domains in the blocked scFvs described in Example 9 is manipulated to add a DNA sequence (SEQ ID NO:8) that would code for the peptide sequence Glu-Asn-Leu-Tyr-Phe-Gln-Gly (SEQ ID NO:7). This peptide sequence is recognized and cleaved by TEV protease.

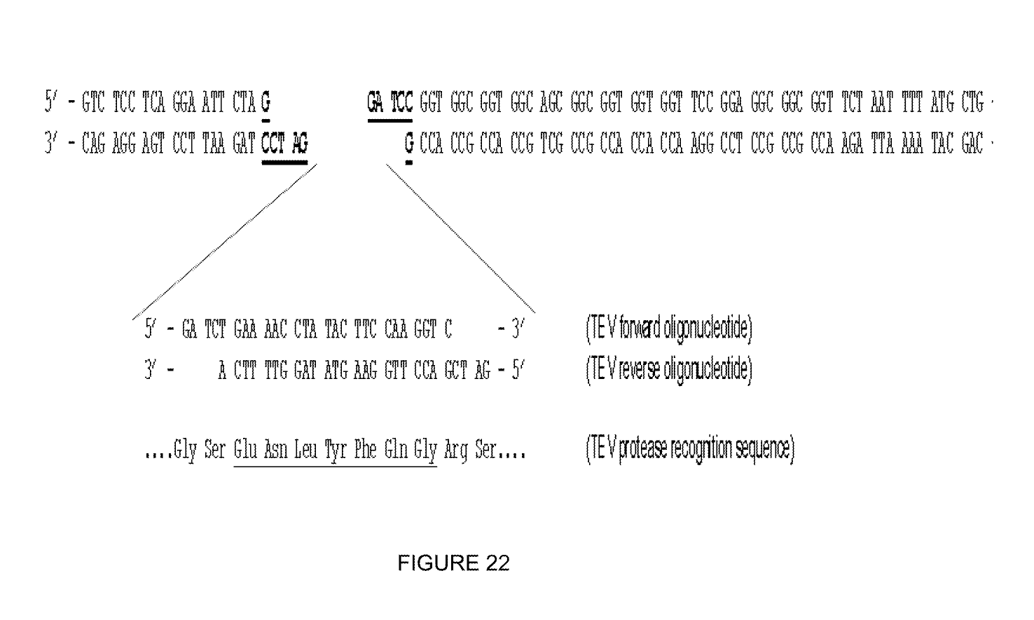

The plasmid pPNL6 is used to construct blocked scFvs as described above. The DNA encoding for the peptide linker segment connecting the V_{H} and V_{L} domains in the blocked scFvs comprises a recognition sequence that is cleaved by BamH1 restriction enzyme. In this example, the DNA comprised a 5′-GGA TCC- sequence and a 3′-CCT AGG- sequence, which is cleaved by BamH1 restriction enzyme as indicated in FIG. 22.

A DNA segment coding for the peptide sequence Glu-Asn-Leu-Tyr-Phe-Gln-Gly and comprising BamH1 compatible ends is joined to the BamH1-cleaved plasmid using standard DNA ligation techniques known in the art. The resulting plasmid comprises a DNA sequence (SEQ ID NO:46) comprising a TEV protease recognition sequence, which when expressed, results in a peptide construct (SEQ ID NO:45) comprising a fluorogen-activating peptide and a blocking peptide linked to the fluorogen-activating peptide through a peptide linker comprising an amino acid sequence that is specifically recognized as a modification substrate by a cognate enzyme, in this example TEV protease.

Peptide constructs produced according to this example comprise a fluorogen-activating peptide (comprising H6-MG V_{H} domain) and a blocking peptide (comprising HL1-TO1 V_{L} domain) linked to the fluorogen-activating peptide through a peptide linker comprising an amino acid sequence that is specifically recognized as a modification substrate by a protease. The constructs may find utility as biosensors for protease activity.

### Example 21

### Construction of a Furin Protease Biosensor

The DNA sequence coding for the peptide linker that covalently links the V_{H} and V_{L} domains in the blocked scFvs described in Example 9 was manipulated to add a DNA sequence (SEQ ID NO:4) that would code for the peptide sequence Arg-Lys-Lys-Arg-Ser (furin short recognition sequence) (SEQ ID NO:3). This peptide sequence is recognized and cleaved by furin protease.

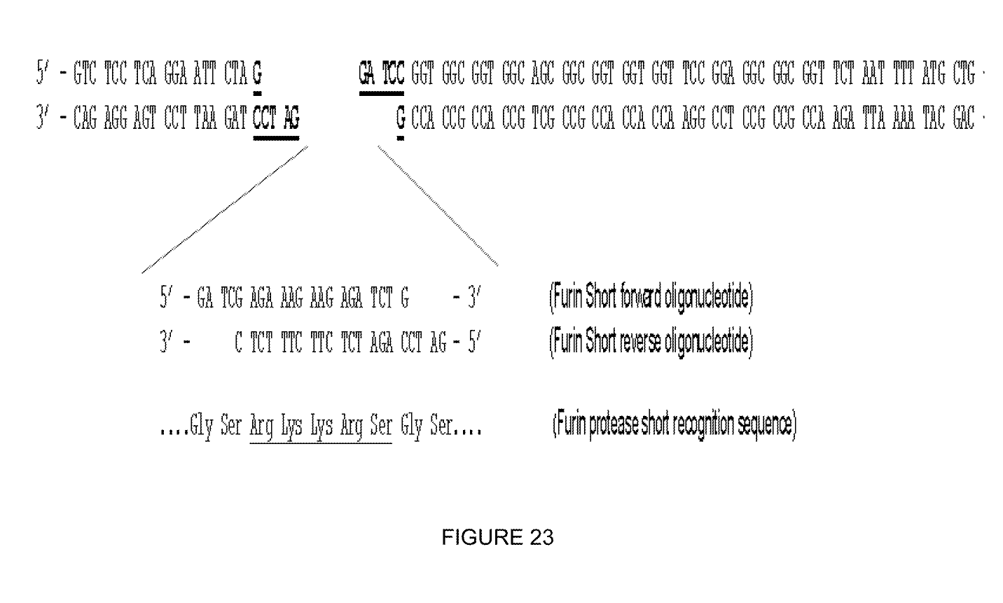

The plasmid pPNL6 was used to construct blocked scFvs as described above. The DNA encoding for the peptide linker segment connecting the V_{H} and V_{L} domains in the blocked scFvs comprised a recognition sequence that was cleaved by BamH1 restriction enzyme. In this example, the DNA comprised a 5′-GGA TCC- sequence and a 3′-CCT AGG- sequence, which is cleaved by BamH1 restriction enzyme as indicated in FIG. 23.

A DNA segment coding for the peptide sequence Arg-Lys-Lys-Arg-Ser and comprising BamH1 compatible ends was joined to the BamH1-cleaved plasmid using standard DNA ligation techniques known in the art. The resulting plasmid comprised a DNA sequence (SEQ ID NO:42) comprising a furin protease short recognition sequence, which when expressed, resulted in a peptide construct (SEQ ID NO:41) comprising a fluorogen-activating peptide and a blocking peptide linked to the fluorogen-activating peptide through a peptide linker comprising an amino acid sequence that was specifically recognized as a modification substrate by a cognate enzyme, in this example furin protease.

The peptide constructs produced in this example comprised a fluorogen-activating peptide (comprising H6-MG V_{H} domain) and a blocking peptide (comprising HL1-TO1 V_{L} domain) linked to the fluorogen-activating peptide through a peptide linker comprising an amino acid sequence that was specifically recognized as a modification substrate by a protease. The constructs find utility as biosensors for furin protease activity.

### Example 22

### Construction of a Furin Protease Biosensor

The DNA sequence coding for the peptide linker that covalently links the V_{H} and V_{L} domains in the blocked scFvs described in Example 9 was manipulated to add a DNA sequence (SEQ ID NO:6) that would code for the peptide sequence Asn-Ser-Arg-Lys-Lys-Arg-Ser-Thr-Ser-Ala (furin long recognition sequence) (SEQ ID NO:5). This peptide sequence is recognized and cleaved by furin protease.

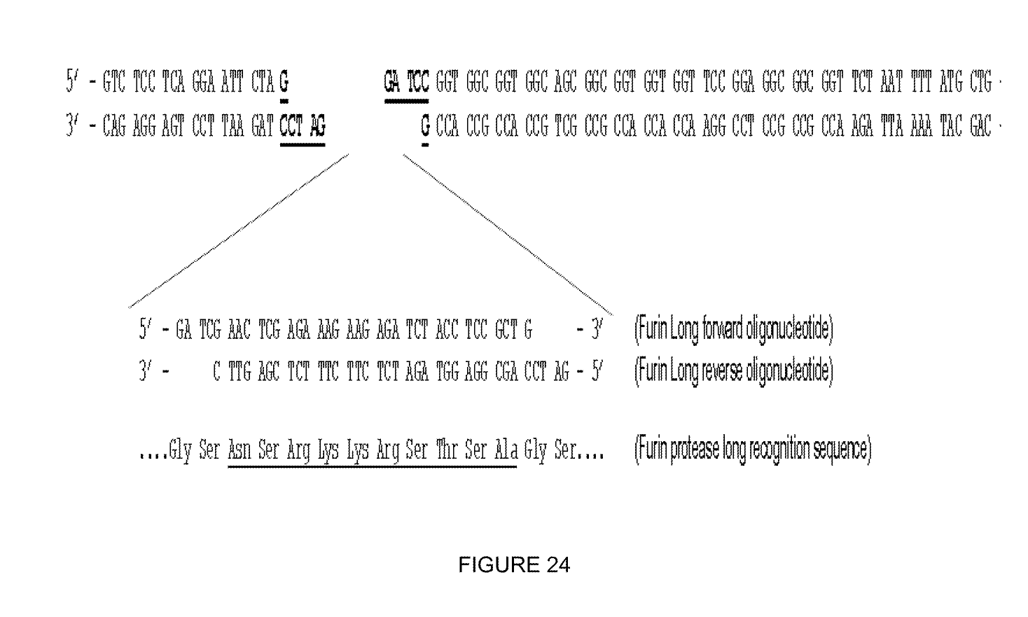

The plasmid pPNL6 was used to construct blocked scFvs as described above. The DNA encoding for the peptide linker segment connecting the V_{H} and V_{L} domains in the blocked scFvs comprised a recognition sequence that was cleaved by BamH1 restriction enzyme. In this example, the DNA comprised a 5′-GGA TCC- sequence and a 3′-CCT AGG- sequence, which was cleaved by BamH1 restriction enzyme as indicated in FIG. 24.

A DNA segment coding for the peptide sequence Asn-Ser-Arg-Lys-Lys-Arg-Ser-Thr-Ser-Ala and comprising BamH1 compatible ends was joined to the BamH1-cleaved plasmid using standard DNA ligation techniques known in the art. The resulting plasmid comprised a DNA sequence (SEQ ID NO:44) comprising a furin protease long recognition sequence, which when expressed, resulted in a peptide construct (SEQ ID NO:43) comprising a fluorogen-activating peptide and a blocking peptide linked to the fluorogen-activating peptide through a peptide linker comprising an amino acid sequence that was specifically recognized as a modification substrate by a cognate enzyme, in this example furin protease.

The peptide constructs produced in this example comprised a fluorogen-activating peptide (comprising H6-MG V_{H} domain) and a blocking peptide (comprising HL1-TO1 V_{L} domain) linked to the fluorogen-activating peptide through a peptide linker comprising an amino acid sequence that was specifically recognized as a modification substrate by a protease. The constructs find utility as biosensors for furin protease activity.

### Example 23

### Isolation and Purification of Furin Biosensors

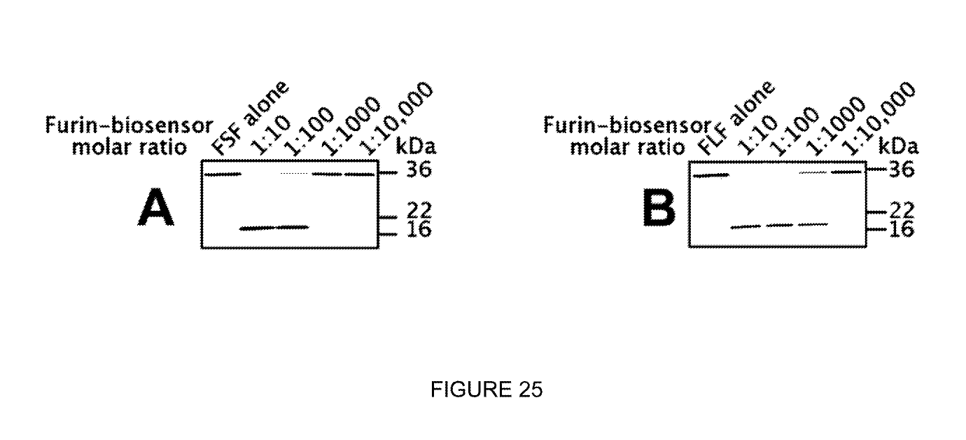

Furin biosensors as described in Examples 21 and 22 were isolated and purified as described in Example 7. The biosensors comprising the short furin recognition sequence (“FSF”) (Example 21) and the long furin recognition sequence (“FLF”) (Example 22) were respectively mixed with purified furin protease under conditions suitable for enzymatic activity (100 mM HEPES buffer pH 7.5, 1 mM CaCl₂, 1 mM beta-mercaptoethanol, for 1 hour at 37° C.) in the following molar ratios: 1:10; 1:100; 1:1000; 1:10000. Coomasie blue stained SDS polyacrylamide electrophoresis gels of the purified furin biosensors treated with furin are presented in FIG. 25 (gel A corresponds to FSF; gel B corresponds to FLF).

Referring to FIG. 25A (FSF), the biosensor without furin treatment was not cleaved, as was expected. Cleavage of the biosensors was complete or nearly complete at 1:10 and 1:100 mixture ratios. The two proteolytic fragments of the biosensor were so close in size that they were not resolved on the SDS gel system. The biosensor was not cleaved at 1:1000 and 1:10000 mixture ratios.

Referring to FIG. 25B (FLF), the biosensor was stable when not contacted with furin. Cleavage of the sensor was complete at ratios of 1:10, 1:100, and 1:1000. No cleavage was apparent at the 1:10000 ratio.

### Example 24

### Kinetic Analysis of Isolated and Purified Furin Biosensors

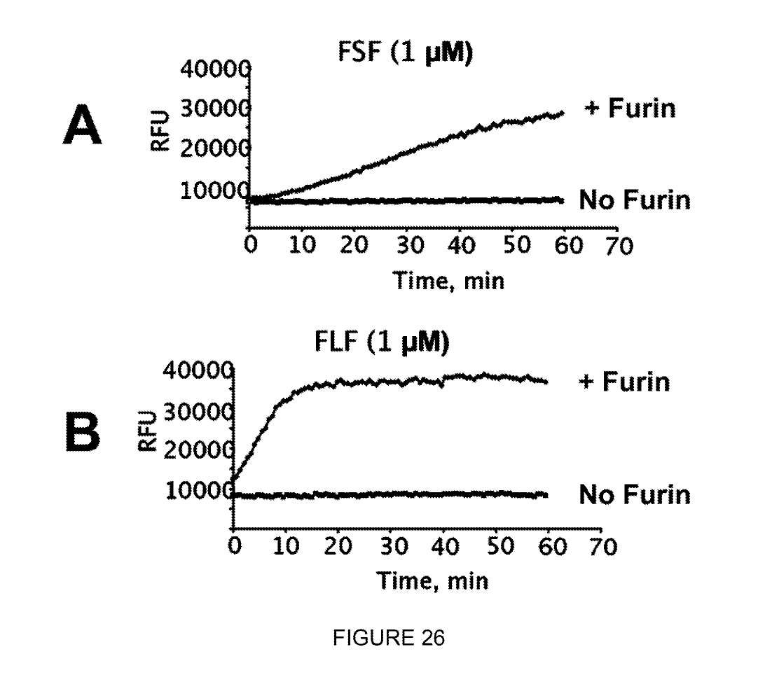

The potential for the biosensors described in Example 23 to be used as a real time assay for detecting the amount of furin protease in a sample was explored by evaluating the kinetics of fluorescence activation for the biosensor in a BioTek Synergy HT Fluorimeter (excitation at 590 nm, emission recorded at 645 nm, the gain (PMT) was 150). 1 μM of short sequence furin biosensor and 1 μM of long sequence furin biosensor were respectively incubated with 0.01 μM furin in 100 mM HEPES pH 7.5, 1 mM CaCl₂, 1 mM beta-mercaptoethanol, 0.1% w/v Pluronic F127, and 100 nM MG fluorogen, for 1 hour at room temperature. FIG. 26 shows the data for the kinetic assays of the activation of the furin protease biosensors (top curves represent biosensor treated with furin, bottom curves represent biosensors without furin contact).

### Example 25

### Construction of an MMP Protease Biosensor

The DNA sequence coding for the peptide linker that covalently links the V_{H} and V_{L} domains in the blocked scFvs described in Example 9 was manipulated to add a DNA sequence (SEQ ID NO:16) that would code for the peptide sequence Val-Met-Arg-Leu-Val-Val (SEQ ID NO:15). This peptide sequence is recognized and cleaved by MMP25 protease.

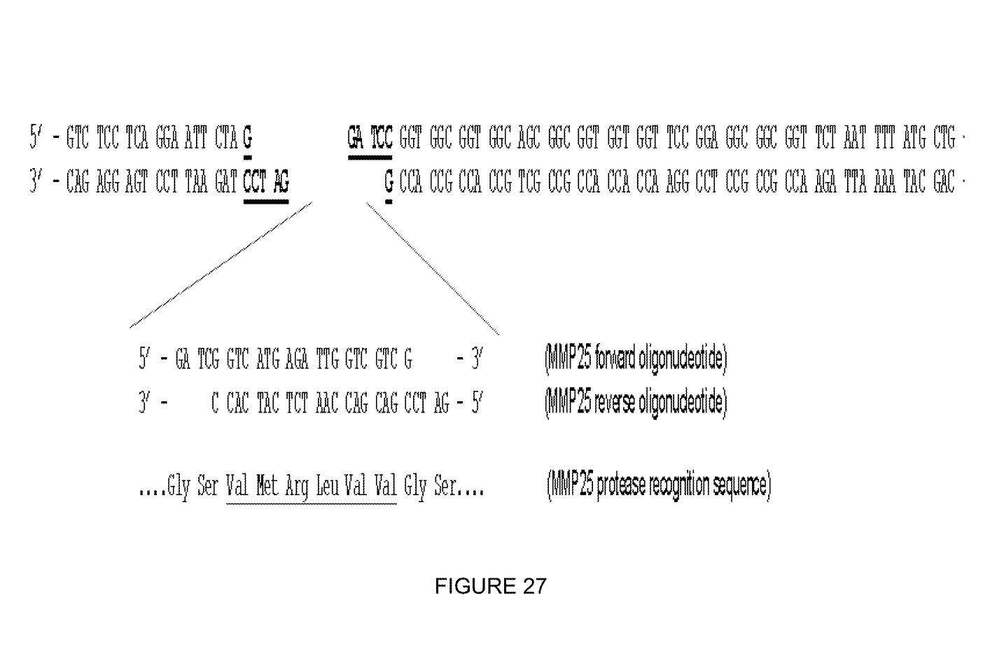

The plasmid pPNL6 was used to construct blocked scFvs as described above. The DNA encoding for the peptide linker segment connecting the V_{H} and V_{L} domains in the blocked scFvs comprised a recognition sequence that was cleaved by BamH1 restriction enzyme. In this example, the DNA comprised a 5′-GGA TCC- sequence and a 3′-CCT AGG- sequence, which was cleaved by BamH1 restriction enzyme as indicated in FIG. 27.

A DNA segment coding for the peptide sequence Val-Met-Arg-Leu-Val-Val and comprising BamH1 compatible ends was joined to the BamH1-cleaved plasmid using standard DNA ligation techniques known in the art. The resulting plasmid comprised a DNA sequence (SEQ ID NO:54) comprising a MMP25 protease recognition sequence, which when expressed, resulted in a peptide construct (SEQ ID NO:53) comprising a fluorogen-activating peptide and a blocking peptide linked to the fluorogen-activating peptide through a peptide linker comprising an amino acid sequence that is specifically recognized as a modification substrate by a cognate enzyme, in this example MMP25 protease.

The peptide constructs produced according to this example comprised a fluorogen-activating peptide (comprising H6-MG V_{H} domain) and a blocking peptide (comprising HL1-TO1 V_{L} domain) linked to the fluorogen-activating peptide through a peptide linker comprising an amino acid sequence that is specifically recognized as a cleavage substrate by MMP25 protease. The constructs may find utility as biosensors for protease activity.

### Example 26

### Construction of a PKA Biosensor

The DNA sequence coding for the peptide linker that covalently links the V_{H} and V_{L} domains in the blocked scFvs described herein is manipulated to add a DNA sequence (SEQ ID NO:18) that codes for the peptide sequence Leu-Leu-Arg-Arg-Ala-Ser-Leu-Gly-Pro (SEQ ID NO:17). This peptide sequence is recognized and phosphorylated by PKA.

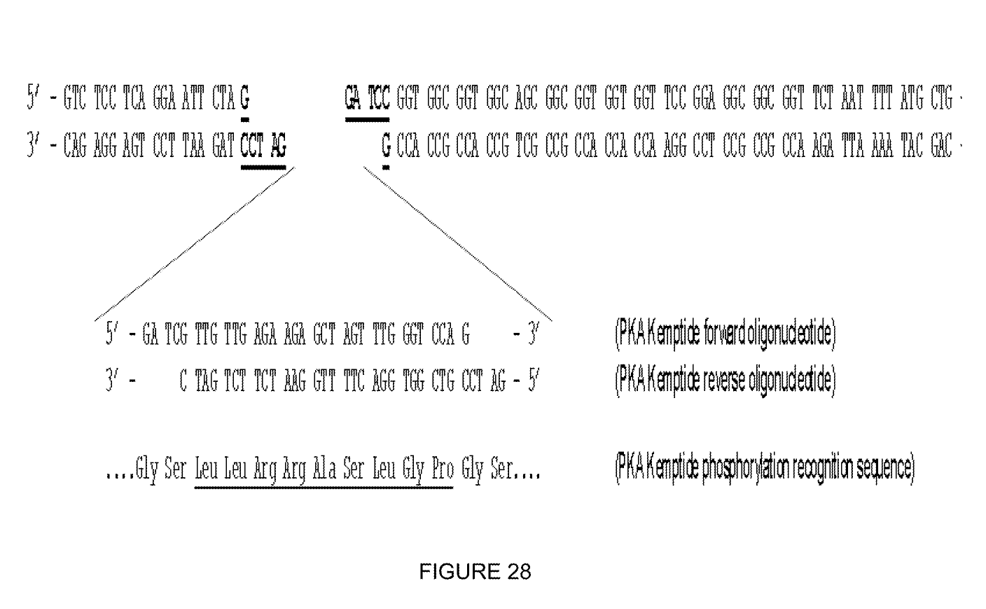

The plasmid pPNL6 is used to construct blocked scFvs as described above. The DNA encoding for the peptide linker segment connecting the V_{H} and V_{L} domains in the blocked scFvs comprises a recognition sequence that is cleaved by BamH1 restriction enzyme. In this example, the DNA comprises a 5′-GGA TCC- sequence and a 3′-CCT AGG- sequence, which may be cleaved by BamH1 restriction enzyme as indicated in FIG. 28.

A DNA segment coding for the peptide sequence Leu-Leu-Arg-Arg-Ala-Ser-Leu-Gly-Pro (SEQ ID NO: 17) and comprising BamH1 compatible ends is joined to the BamH1-cleaved plasmid using standard DNA ligation techniques known in the art. The resulting plasmid comprises a DNA sequence (e.g., SEQ ID NO:56) comprising a PKA recognition sequence, which when expressed, results in a peptide construct (e.g., SEQ ID NO:55) comprising a fluorogen-activating peptide and a blocking peptide linked to the fluorogen-activating peptide through a peptide linker comprising an amino acid sequence that is specifically recognized as a modification substrate by a cognate enzyme, in this example PKA.

Peptide constructs produced according to this example comprise a fluorogen-activating peptide and a blocking peptide linked to the fluorogen-activating peptide through a peptide linker comprising an amino acid sequence that is specifically recognized as a phosphorylation substrate by PKA. The constructs may find utility as biosensors for phosphorylation activity.

### Example 27

### Construction of a PKA Biosensor

A PKA biosensor as described in Example 26 is modified to further comprise 14-3-3τ peptide covalently linked to the C-terminal end of the biosensor. The biosensor may comprise a fluorogen-activating peptide comprising the L5.1-MG V_{L} domain and a blocking peptide comprising the HL4-MG V_{H} domain. The L5.1-MG V_{L} domain and the HL4-MG V_{H} domain are linked through a peptide linker comprising a (Gly₄Ser)₃ sequence and a Leu-Leu-Arg-Arg-Ala-Ser-Leu-Gly-Pro sequence (SEQ ID NO: 17) such that the L5.1-MG V_{L} domain is on the C-terminal end of the peptide construct and the HL4-MG V_{H} domain is on the N-terminal end of the peptide construct.

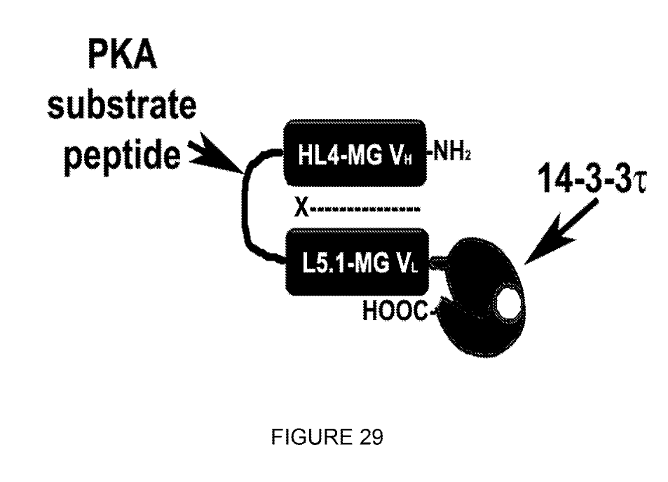

The plasmid comprising the DNA coding for the biosensor is cut using appropriate restriction enzymes after the DNA sequence coding for the L5.1-MG V_{L} domain. A DNA sequence coding for 14-3-3τ peptide is ligated into the plasmid such that when expressed, the resulting protein construct comprises a 14-3-3τ peptide on the C-terminal end of the L5.1-MG V_{L} domain. A peptide construct produced according to this example is illustrated in FIG. 29.

Peptide constructs produced according to this example comprise a fluorogen-activating peptide and a blocking peptide linked to the fluorogen-activating peptide through a peptide linker comprising an amino acid sequence that is specifically recognized as a phosphorylation substrate by PKA. The 14-3-3τ peptide may complex with the peptide linker when it is phosphorylated by PKA. The constructs may find utility as biosensors for phosphorylation activity.

### Example 28

### Construction of a Biosensor to Detect H3-K56 Acetylation Activity

The DNA sequence coding for the peptide linker that covalently links the V_{H} and V_{L} domains in the blocked scFvs described herein is manipulated to add a DNA sequence (SEQ ID NO:20) that codes for the peptide sequence Ile-Arg-Arg-Phe-Gln-Lys-Ser-Thr-Asp-Leu-Leu (SEQ ID NO:19). This peptide sequence is recognized and acetylated by H3-K56 acetyltransferase.

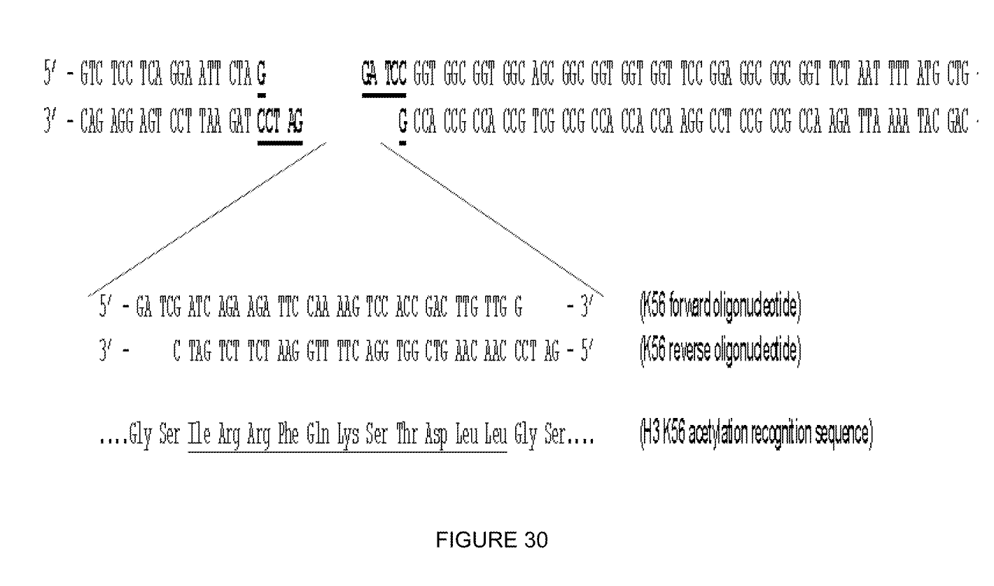

The plasmid pPNL6 is used to construct blocked scFvs as described above. The DNA encoding for the peptide linker segment connecting the V_{H} and V_{L} domains in the blocked scFvs comprises a recognition sequence that is cleaved by BamH1 restriction enzyme. In this example, the DNA comprises a 5′-GGA TCC- sequence and a 3′-CCT AGG- sequence, which is cleaved by BamH1 restriction enzyme as indicated in FIG. 30.

A DNA segment coding for the peptide sequence Ile-Arg-Arg-Phe-Gln-Lys-Ser-Thr-Asp-Leu-Leu (SEQ ID NO:19) and comprising BamH1 compatible ends is joined to the BamH1-cleaved plasmid using standard DNA ligation techniques known in the art. The resulting plasmid comprises a DNA sequence (e.g., SEQ ID NO:58) comprising a H3-K56 acetyltransferase recognition sequence, which when expressed, results in a peptide construct (e.g., SEQ ID NO:57) comprising a fluorogen-activating peptide and a blocking peptide linked to the fluorogen-activating peptide through a peptide linker comprising an amino acid sequence that is specifically recognized as a modification substrate by a cognate enzyme, in this example H3-K56 acetyltransferase.

Peptide constructs produced according to this example comprise a fluorogen-activating peptide and a blocking peptide linked to the fluorogen-activating peptide through a peptide linker comprising an amino acid sequence that is specifically recognized as an acetylation substrate by H3-K56 acetyltransferase. The constructs may find utility as biosensors for acetylation activity.

### Example 29

### Construction of a Biosensor to Detect H3-K56 Acetylation Activity

A H3-K56 acetyltransferase biosensor as described in Example 28 is modified to further comprise bromo-domain peptide covalently linked to the C-terminal end of the biosensor. The biosensor may comprise a fluorogen-activating peptide comprising the L5.1-MG V_{L} domain and a blocking peptide comprising the HL4-MG V_{H} domain. The L5.1-MG V_{L} domain and the HL4-MG V_{H} domain are linked through a peptide linker comprising a (Gly₄Ser)₃ sequence and a Ile-Arg-Arg-Phe-Gln-Lys-Ser-Thr-Asp-Leu-Leu sequence (SEQ ID NO:19) such that the L5.1-MG V_{L} domain is on the C-terminal end of the peptide construct and the HL4-MG V_{H} domain is on the N-terminal end of the peptide construct.

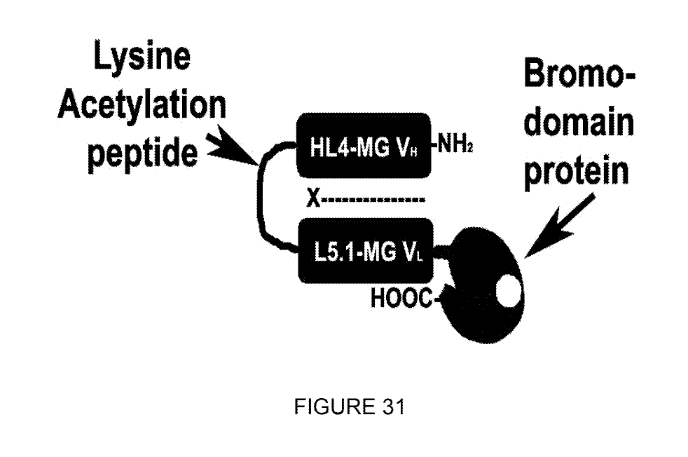

The plasmid comprising the DNA coding for the biosensor is cut using appropriate restriction enzymes after the DNA sequence coding for the L5.1-MG V_{L} domain. A DNA sequence coding for bromo-domain peptide is ligated into the plasmid such that when expressed, the resulting protein construct comprises a bromo-domain peptide on the C-terminal end of the L5.1-MG V_{L} domain. A peptide construct produced according to this example is illustrated in FIG. 31.

Peptide constructs produced according to this example comprise a fluorogen-activating peptide and a blocking peptide linked to the fluorogen-activating peptide through a peptide linker comprising an amino acid sequence that is specifically recognized as an acetylation substrate by H3-K56 acetyltransferase. The bromo-domain peptide may complex with the peptide linker when it is acetylated by H3-K56 acetyltransferase. The constructs may find utility as biosensors for acetylation activity.

Improvements with a Unique Convertible Linker:

To improve efficiency and lower costs associated with creating the blocking peptide constructs described above, a unique platform was created to allow customization of the biosensor constructs. The platform includes a convertible linker segment that may be genetically manipulated to efficiently insert a variety of DNA or nucleotide sequences between the fluorogen activating peptide and the blocking peptide to create customized target DNA or nucleotide sequences coding for a variety of desired target amino acid sequences. As a result, the platform provides an efficient and cost effective means to enhance the family of the disclosed biosensors.

In general, the disclosed biosensor system may be employed to efficiently insert a desired linker sequence between two peptides. For example, beginning with an initial plasmid comprising a first scFv coding sequence and a second scFv coding sequence, wherein the first scFv sequence encodes one of a fluorogen activating peptide and a blocking peptide and the second scFv sequence encodes the other of the fluorogen activating peptide and the blocking peptide, and wherein the first scFv and the second scFv are linked by a linker comprising a first restriction enzyme cleavage site that is cleavable by a first restriction enzyme, the initial plasmid DNA may be digested with the first restriction enzyme. Preferably, the first restriction enzyme that is selected is one that cleaves DNA to produce an overhang sequence at each of a first and a second end of the cleaved linearized plasmid DNA. An added DNA segment comprising overhang sequences complementary to the overhang sequences at each of the first and second ends of the linearized DNA of the initial plasmid may be ligated between such respective first and second ends to again circularize the DNA to form a modified plasmid. The DNA segment inserted within the linker may comprise a second restriction enzyme cleavage site and a third restriction enzyme cleavage site, each of which is positioned to flank a target DNA sequence position. That is, the target DNA sequence is positioned between the second and third restriction enzyme cleavage sites. The second restriction enzyme cleavage site may be cleavable by a second restriction enzyme and the third restriction enzyme cleavage site may be cleavable by a third restriction enzyme. Preferably, the second and third restriction enzymes are restriction enzymes that cleave DNA to produce different overhang sequences. As described in more detail below, in various embodiments, the first and second restriction enzymes may be the same restriction enzyme wherein the restriction enzyme is associated with multiple cleavage sites. The circularized modified plasmid may be digested with the second and third restriction enzymes to produce a linearized plasmid DNA comprising different overhang sequences at a first end and a second end of the cleaved linearized DNA of the modified plasmid. According to various embodiments, a target DNA segment comprising overhang sequences complementary to the different overhang sequences of the first and second ends of the linearized plasmid DNA of the modified plasmid may be ligated between such respective first and second ends of the linearized DNA of the modified plasmid to re-circularize it and form a further modified plasmid that contains the target DNA sequence. A portion of the inserted DNA segment extending between the second and third restriction enzyme cleavage sites preferably comprises a target DNA sequence, e.g., a DNA sequence encoding an amino acid sequence that is a target of the molecule or condition of interest. In various embodiments, the target DNA sequence may code for an amino acid sequence comprising a protease recognition sequence as described herein.

The new convertible linker sequence may generally extend between the first scFv coding sequence and the second scFv coding sequence, as described above, and may be efficiently convertible to produce customized biosensors. For example, a plasmid comprising the new convertible linker positioned between the first and second scFv coding sequences may be digested with the second and third restriction enzymes to linearize the plasmid and thereby release an insert sequence positioned where a desired target DNA sequence in the new convertible linker is to be added. The digested plasmid may comprise a first end and a second each comprising different overhang sequences corresponding to respective digestion products of the second and third restriction enzymes. A DNA segment comprising a desired target sequence flanked by a first end compatible with the first overhang sequence of the digested plasmid and a second end compatible with the second overhang sequence of the digested plasmid may be ligated to the DNA of the linearized plasmid to produce a circularized plasmid comprising the first scFv coding sequence, the second scFv coding sequence, and the convertible linker therebetween wherein the converted linker comprises a converted target DNA sequence. Accordingly, the platform may be used to efficiently exchange different target sequences within the convertible linker to produce biosensors comprising customized linkers.

Among the beneficial aspects of the unique biosensor system disclosed herein is reduction of false positives and otherwise costly, yet customary, trial and error protocols. Because suitable DNA molecules must comprise the correct compatible overhang sequences with respect to the overhang sequences of the plasmid backbone, whatever colonies appear after ligation and transformation carry plasmids with an insertion of the target DNA sequence. Further, because the target coding DNA sequence has different overhang sequences on each end, the target coding DNA sequence will be ligated and circularize the plasmid in a predetermined orientation by design. Thus, for example, if the overhang sequences at the first end and the second end are the same, only 50% of the successful insertions would be expected to insert in the correct orientation. However, using the platform biosensor system of the present disclosure, 100% of the inserted target DNA sequences would be expected to insert in the correct orientation. Further, because the inserted DNA segment comprising the target DNA sequence comprises a different overhang sequences on each end, one and only one target molecule may be ligated into the plasmid backbone.

The platform enables the creation of a variety of desired biosensors, which may comprise a convertible linker sequence that extends between the first scFv coding sequence and the second scFv coding sequence, wherein the first scFv coding sequence codes for a fluorogen activating peptide and the second scFv coding sequence codes for a blocking peptide. In certain preferred embodiments, one of the fluorogen-activating peptide and the blocking peptide comprises a variable heavy chain domain of an antibody and the other peptide comprises a variable light chain domain of a different antibody. The convertible linker sequence may comprise a first and a second restriction enzyme cleavage site. In one embodiment, the convertible linker sequence comprises an insertion portion that may be positioned between the first and second restriction enzyme cleavage sites. Digestion with the first and second restriction enzymes may result in removal of a target sequence. Subsequently, a different target DNA sequence comprising overhang sequences compatible with the first and second restriction enzyme cleavage sites may be ligated with the cleavage sites to circularize the plasmid DNA. For example, the target DNA sequence may comprise a modification or cleavage substrate that is cleaved due to an activity associated with a molecule or condition of interest. Cleavage of the peptide linker may allow interaction between a fluorogen and the fluorogen activating peptide to modulate a fluorescence signal produced by the fluorogen.

The platform biosensor system will be described in more detail in the following examples.

### Example 30

### Synthesis of Platform Biosensor System

A linker extension was designed and inserted into the linking construct comprising the nucleotide sequence (SEQ ID NO:2) depicted in FIG. 11A, beginning with the first “GGT” and ending with “TCT” and coding the peptide linker amino acid sequence (Gly₄Ser)₃ (SEQ ID NO:1), which is also depicted in FIG. 11A beginning with the third “Gly” and ending with the sixth “Ser”. The gene segment shown in FIG. 11A is part of the plasmid pPNL6. The plasmid pPNL6 was used to construct blocked scFvs as described above. The DNA coding for the peptide linker segment connecting the V_{H} and V_{L} domains in the blocked scFvs comprised a recognition sequence that was cleaved by BamH1 restriction enzyme to produce overhang sequences comprising a 5′-GGA TCC- sequence and a 3′-CCT AGG- sequence, as indicated in the top portion of FIG. 11B. The 5′ top strand includes therein a sequence coding for (Gly₄Ser)₃.

A new linker comprising a target DNA sequence (SEQ ID NO:10) coding the target peptide sequence for the Human Rhinovirus 3C (“HRV-3C”) protease Leu-Glu-Val-Leu-Phe-Gln-Gly-Pro (SEQ ID NO:9) was developed with increasing efficiency in mind. This target peptide sequence is recognized and cleaved by the HRV-3C protease between the Gln and Gly residues. In contrast to the construction of the blocked scFv having an HRV-3C protease substrate described in Example 10 and depicted in FIG. 11B, the new linker comprised a linker extension having BamH1 compatible overhang sequences by design.

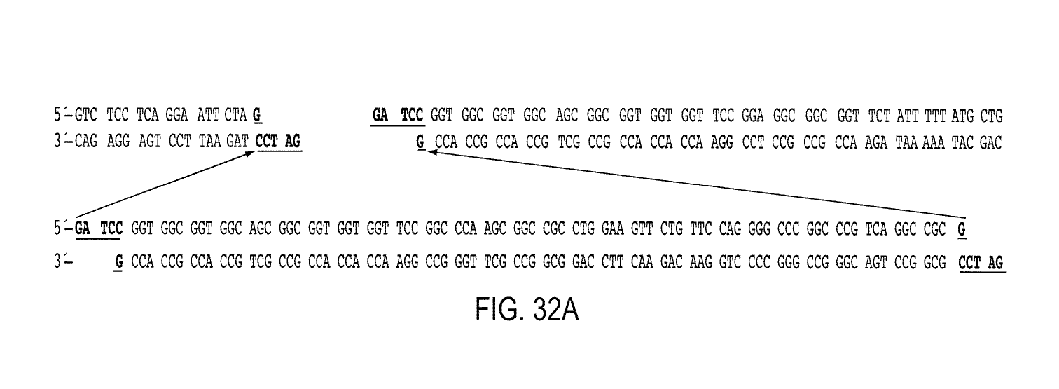

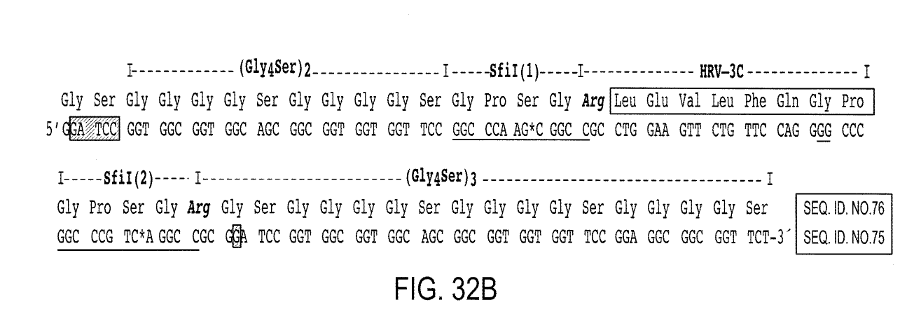

FIGS. 32A-32B illustrate the development of the platform biosensor system and the creation of a biosensor comprising a convertible linker sequence. FIG. 32A depicts BamH1 specific digestion of the linker sequence (upper sequence) that extends between a first scFv coding sequence and a second scFv coding sequences, e.g., similar to the biosensor gene of Example 10. The BamH1 specific digestion produces BamH1 overhang sequences comprising 4-base, 5′ overhangs (underlined and bolded). The lower sequence illustrates two complementary DNA fragments (Top: (SEQ ID NO:59); Bottom: (SEQ ID NO:60)) that were synthesized and annealed using standard annealing techniques. See for example, (http://www.sigmaaldrich.com/life-science/custom-oligos/custom-dna/learning-center/annealing-oligos.html (Mar. 13, 2013). The annealed complementary DNA fragments comprise a linker extension that was ligated into the cleaved plasmid in the BamH1 site of the peptide linker region, e.g., as described in Example 10, between the V_{H} and V_{L} domains. The linker extension comprises a sequence coding for the HRV-3C peptide recognition sequence positioned between the BamH1 compatible ends (underlined and bolded).

The linker extension was ligated into the construct using standard protocols. See, for example, (http://www.addgene.org/plasmid_protocols/DNA_ligation/ [Mar. 13, 2013]) Successful insertions where sequences were sequenced to ensure correct orientation. As stated above, FIG. 32B illustrates one embodiment of a convertible linker sequence (SEQ ID NO:75) (and expressed peptide (SEQ ID NO:76)). The linker extension extends between the shaded “GA TCC” sequence and the shaded “G” nucleotide and, starting from the 5′ end of the linker extension, the linker extension comprises a first sticky end comprising a compatible BamH1 sequence (shaded), a sequence coding the peptide sequence (Gly₄Ser)₂, a first SfiI recognition sequence “SfiI(1)” (SEQ ID NO:61) (underlined, “*” identifies cleavage point), a target DNA sequence (SEQ ID NO:10) positioned within a target DNA coding portion wherein the target DNA sequence codes for the HRV-3C protease recognition (SEQ ID NO:9) (boxed), a second SfiI recognition sequence “SfiI(2)” (SEQ ID NO:62)) (underlined, “*” identifies cleavage point) that differs from SfiI(1), a second overhang comprising a compatible BamHI sequence (shaded, only the 5′ strand is shown), and, after insertion of the linker extension the 3′ portion of the convertible linker further comprises a second sequence encoding the peptide sequence (Gly₄Ser)₃. The resulting plasmid comprised a DNA sequence (SEQ ID NO: 10) comprising an HRV-3C protease recognition sequence, which when expressed, resulted in a peptide construct (SEQ ID NO:9) comprising a fluorogen-activating peptide and a blocking peptide linked to the fluorogen-activating peptide through a peptide linker comprising an amino acid sequence that was specifically recognized as a modification substrate by a cognate enzyme, in this example HRV-3C protease.

### Example 31

### Formation of Biosensor Expression Vector with HRV-3C Recognition Site

To prepare the biosensor, the recombinant plasmid pPNL6 was digested with NheI and NotI and the digestion products were separated by gel electrophoresis according to standard protocols known in the art. See, for example, (http://www.addgene.org/plasmid_protocols/gel_purification/ [Mar. 13, 2013]). The NheI-NotI biosensor gene fragment was subsequently cloned into a protein expression plasmid derived from plasmid pET21a, which placed the biosensor gene as a fusion gene under the control of the lac operon, T7 RNA polymerase expression system. See (http://www.ncbi.nlm.nih.gov/pubmed/3537305, accessed Feb. 25, 2013). Briefly, the biosensor gene fragment was cloned in frame to the C-terminus of an artificial gene segment comprising an N-terminal His₁₀ sequence, fused in frame with the Glutathione S-Transferase gene (from Schistosoma japonicum, e.g., pGEX vectors (http://www.synthesisgene.com/vector/pGEX-4T-2.pdf, accessed Feb. 24, 2013), and fused in frame with a DNA sequence that codes for the HRV-3C protease recognition site (LeuGluValLeuPheGln/GlyPro). The forward slash symbol, “/”, marks the cleavage site of the HRV-3C protease.

### Example 32

### Transformation of the Expression Vector

The biosensor expression vector was used to transform E. coli Rosetta-gami™ 2 competent cells. The transformed cells were grown under antibiotic selective conditions to mid log density in Luria Broth at 37° C. and then shifted to 20° C. The culture was induced with 0.5 mM IPTG (iso-propyl thiogalactoside) and grown an additional 16 hours. The cells were harvested by centrifugation and the cell pellet was resuspended in a volume of lysis buffer (0.1M Tris-HCl pH7.5+0.75M NaCl+0.1% TRITON X-100+0.05% TWEEN 20) equal to 0.1× culture volume and frozen at −20° C. The frozen pellets were subsequently thawed and cell suspension was homogenized in an Avastin homogenizer for 10 minutes. The resulting cell lysate was clarified by high-speed centrifugation. Imidazole was added to the supernatant fraction to a concentration of 50 mM. Resuspended Ni⁺²-NTA slurry was added to the clarified cell lysate at 1.2 ml per 500 ml of original cell culture volume and the suspension was allowed to slowly mix at 4° C. for 2 hours. The suspension was subsequently poured into a disposable column to collect the resin and the flow-through liquid was passed once over the settled Ni⁺²-NTA resin. The resin was washed with 10 column volumes of buffer A (50 mM Tris-Cl pH7.5, 750 mM NaCl, 50 mM imidazole, 0.1% T×100, 0.05% TWEEN 20) and then resuspended in 0.6 ml volume of buffer A containing (250 micrograms of HRV-3C protease). The resin solution was gently mixed overnight at 4° C. An additional 80 microliters of Ni⁺²-NTA resin was added to the column for an additional hour of incubation. The resin was allowed to settle in the column and the flow through liquid containing the first fraction of biosensor protein was collected. The column was washed with four additional 0.3 ml portions of buffer A, which were collected and added to the first fraction. The preparation yielded approximately 2 to 3 ml of biosensor protein preparation containing 3-5 mg of protein per 500 ml original cell culture volume.

The new convertible linker includes a number of beneficial features. For example, the convertible linker contains two different SfiI restriction sites, as shown in FIG. 32B (underlined), flanking the DNA sequence coding for the peptide sequence recognized by HRV-3C protease, as shown in FIG. 32B (boxed). Thus, the new convertible linker sequence is positioned between the V_{H} and V_{L} domains of the biosensor gene in the following order: V_{H} domain-BamH1-(Gly₄Ser)₂-SfiI(1)-HRV-3C target-SfiI(2)-BamH1-(Gly₄Ser)₃-V_{L} domain

The SfiI restriction enzyme recognizes a sequence motif characterized by 5′-GGCCNNNN̂NGGCC (SEQ ID NO:74), where N can be any of the 4 bases G, A, T, or C. As those having skill in the art will appreciate, there are 1024 (4⁵) different sequences that may be cleaved by SfiI, which may leave 64 (4³) different 3-base overhang sequences. In the present disclosure, the SfiI sites, SfiI(1) (SEQ ID NO:61) and SfiI(2) (SEQ ID NO:62) were selected such that when the convertible linker sequence is cleaved with SfiI, the target DNA sequence portion disposed between the SfiI cleavage sites is excised from the biosensor gene, e.g., the HRV-3C target site is separated from the biosensor and different, non-complementary overhang sequences are generated on the first and second ends of the remaining plasmid backbone.

### Example

### 33. Using the Platform Biosensor System to Efficiently Convert the Convertible Linker

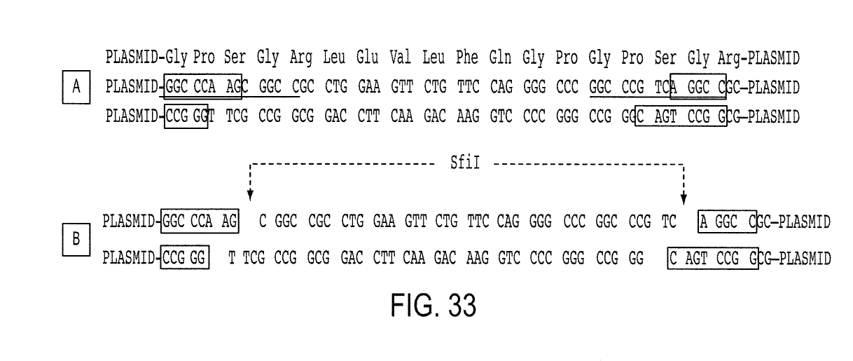

As those having skill in the art will appreciate, the disclosed platform biosensor system provides an efficient mechanism to convert and hence customize the convertible linker for use in diverse applications. FIG. 33 illustrates an example of how the platform biosensor system may be used to manipulate the convertible linker to efficiently convert the biosensor to specify a new target.

a. Creating an MMP2 Protease Biosensor

The platform biosensor system of Example 30 was manipulated to efficiently convert the convertible linker to include a different target DNA sequence. In this example, the convertible linker was manipulated to add a DNA sequence (SEQ ID NO:16) that would code for the peptide sequence Val-Met-Arg-Leu-Val-Val (SEQ ID NO:15). This peptide sequence is recognized and cleaved by MMP25 protease.

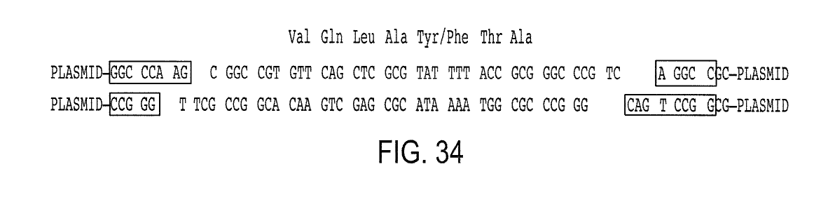

Starting with the biosensor gene and the convertible linker of Example 30 cloned into an expression vector such as that described in Example 31, the plasmid DNA was digested to completion with the restriction enzyme SfiI. FIG. 33 shows how the convertible linker may be manipulated to efficiently exchange target DNA sequences. The convertible linker preferably comprises a first SfiI(1) recognition sequence (SEQ ID:61) and a second recognition sequence SfiI(2) (SEQ ID:62) (both SfiI sequences are underlined in FIG. 33). Accordingly, digestion with SfiI excised the target DNA sequence for the HRV-C3 protease from the convertible linker Thus, digestion with SfiI linearized the plasmid DNA and created two different overhang sequences. The plasmid backbone was isolated from excised HRV-C3 target sequence via gel electrophoresis using standard protocols known in the art, as referenced above. The plasmid backbone was cut from the gel and purified by standard means. Two complimentary oligonucleotides (Top: SEQ ID NO: 64; Bottom: SEQ ID NO:65), shown in FIG. 34, comprising SfiI(1) (SEQ ID:61) and SfiI(2) (SEQ ID:62) complimentary 3′ overhang sequences flanking the target DNA sequence for the MMP protease were generated and annealed in STE buffer (10 mM Tris, 1 mM EDTA pH8.0, 0.1M NaCl) to generate the double-stranded oligonucleotide shown in FIG. 34. The MMP2 protease cleaves the recognition sequence between Tyr-Phe, indicated by the forward slash symbol, “/”, in FIG. 34. The plasmid DNA and the oligonucleotides were mixed at equal molar ratio in DNA ligation buffer. DNA ligase was added and the ligation reaction was incubated at room temperature for 30 minutes. Circularized plasmids were used to transform E. coli to achieve approximately 100 colonies on a single plate. No colonies appeared on control plates containing the same ligation reaction done without added oligonucleotide. The next day, 2 to 3 colonies were selected and a miniprep was performed using a standard protocol. The DNA was sent for sequencing using a sequencing primer that was specific for the rightward sequencing from the middle of the V_{H} domain of the biosensor gene. The sequencing was used to confirm successful insertion of the desired target sequence. The successfully inserted target sequence was made and purified as described above in Example 32.

b. Incorporation of Arginine Amino Acid Codon as a Built-in Positive Control

The new platform biosensor system preferably comprises a convertible linker having an arginine amino acid codon flanking the target DNA sequence insertion position. Thus, irrespective of the inserted target sequence, this embodiment of the convertible linker will code for an arginine amino acid for all biosensor proteins manufactured. For protease biosensors, this arginine amino acid provides a ubiquitous cleavage site for the enzyme trypsin, which cleaves after the basic amino acids arginine and lysine. Thus, treating any protease biosensor manufactured with arginine containing embodiment of the linker system with trypsin will result in cleavage of the linker, allowing the blocking and activating domains to separate. This will generate a fluorescent signal in the presence of the cognate dye for the activating domain. Consequently, the arginine amino acid provides a “built-in” positive control sequence in each protease biosensor, e.g., all biosensors can be activated by trypsin proving that the biosensor is capable of a fluorescent signal in the presence of the target protease. This allows all the biosensors manufactured with the arginine residue to be tested with trypsin.

### Example 34

### Creation of a Protease Biosensor for Botulism Neurotoxin Type A

Botulism Neurotoxin type A (BoNT-A) is a zinc metalloproteinase that cleaves one peptide bond in the SNARE protein SNAP-25 (synaptosome-associated protein of 25 kDa). A peculiarity of BoNT-A distinguishing it from conventional proteases is that it requires an extended substrate segment for optimal catalytic activity. (Vaidyanathan, V. V., Yoshino, K., Jahnz, M., Dorries, C., Bade, S., Nauenburg, S., Niemann, H., and Binz, T., Proteolysis of SNAP-25 isoforms by botulinum neurotoxin types A, C, and E: domains and amino acid residues controlling the formation of enzyme-substrate complexes and cleavage, J Neurochem, 72, 327-337 (1999); Humeau, Y., Doussau, F., Grant, N. J., and Poulain, B., How botulinum and tetanus neurotoxins block neurotransmitter release, Biochimie, 82, 427-446 (2000); Washbourne, P., Pellizzari, R., Baldini, G., Wilson, M. C., and Montecucco, C. Botulinum neurotoxin types A and E require the SNARE motif in SNAP-25 for proteolysis, FEBS letters 418, 1-5 (1997); Sikorra, S., Henke, T., Galli, T., and Binz, T., Substrate recognition mechanism of VAMP/synaptobrevin-cleaving clostridial neurotoxins, The Journal of biological chemistry, 283, 21145-21152 (2008)) This extended substrate sequence is required because the enzyme surface has “exosites” that bind and anchor the substrate to the enzyme. This means that, in general, small peptide substrates will bind poorly to the enzyme, thus making the development of chromogenic or fluorogenic artificial substrates difficult.

The ability of the disclosed FAP-based biosensor platform to accept target sequences of any size has been used to develop a fluorescent substrate for BoNT-A. Three different biosensor genes were constructed using the new biosensor linker platform cloned into a protein expression vector.

a. Synthesis of a 17-Amino Acid SNAP25 Target Sequence for BoNT-A Protease

Complementary oligonucleotide sequences (Top: SEQ ID NO: 66; Bottom: SEQ ID NO: 67) purchased from IDT were annealed to generate a double-stranded oligonucleotide. This oligonucleotide was ligated into the convertible linker of the new designer biosensor platform in a protein expression vector as per the method described above in Example 31. Briefly, the oligonucleotide includes 3′ overhangs complementary to the unique SfiI sites (SfiI(1): (SEQ ID NO:61); SfiI(2):(SEQ ID NO:62), as previously described, which flank a sequence that codes for the 17 amino acid sequence:

Thr-Arg-Ile-Asp-Glu-Ala-Asn-Gln-/-Arg-Ala-Thr-
Lys-Met-Leu-Gly-Ser-Gly-Gly

(SEQ ID NO:68) that comprises the C-terminal 17 amino acids of SNAP25. The forward slash symbol, “/”, marks the cleavage site of BoNT-A protease.

b. Synthesis of a 61-Amino Acid SNAP25 Target Sequence for BoNT-A Protease

The DNA coding for the C-terminal 61 amino acids (SEQ ID NO:71) of SNAP25 (approximately 183 nucleotides in length) was made by PCR from a SNAP25 cDNA clone template (NCBI Reference Sequence: NM_{—}003081.3). An SfiI site “SfiI(3)” (5′GGCCCAAĜCGGCC) (SEQ ID NO:63) was added to the desired 5′ end of the PCR product and the SfiI(2) site (SEQ ID NO:62), as previously described, was added to the desired 3′ end of the PCR product by primer design. Both SfiI-containing primers (Forward: SEQ ID NO:69; Reverse: SEQ ID NO:70) were designed to create an open reading frame between the SfiI sites in a compatible manner with the new designer biosensor platform and in the proper reading frame. The PCR product was cleaved with SfiI to generate the desired compatible 3′ overhang sequences, and the SfiI cleavage product was ligated into the new biosensor platform within a protein expression vector as per the method described above with respect to Example 31. The PCR DNA fragment codes for the 61 amino acid sequence:

MDENLEQVSGIIGNLRHMALDMGNEIDTQNRQIDRIMEKADSNKTRIDE
ANQ/RATKMLGSG

(SEQ ID NO:71) that comprises the C-terminal 61 amino acids of SNAP25. The forward slash symbol, “/”, marks the cleavage site of BoNT-A protease.

c. Synthesis of a 206 Amino Acid SNAP25 Target Sequence for BoNT-A Protease

The DNA coding for the entire 206 amino acids of SNAP25 (SEQ ID NO:72), which comprises approximately 618 nucleotides in length, was made by PCR from a SNAP25 cDNA clone template (NCBI Reference Sequence: NM_{—}003081.3). The SfiI(3) site (SEQ ID NO:63) was added to the desired 5′ end of the PCR product and the SfiI(2) site (SEQ ID NO:62) was added to the desired 3′ end of the PCR product by primer design. Both SfiI-containing primers (Forward: SEQ ID NO:73; Reverse: SEQ ID NO:70) were designed to create an open reading frame between the SfiI sites in a compatible manner with the new biosensor platform and in the proper reading frame. The PCR product was cleaved with SfiI to generate the desired compatible 3′ overhang sequences, and the SfiI cleavage product was ligated into the new biosensor platform within a protein expression vector as per the method described above with respect to Example 31. The PCR DNA fragment codes for the 206 amino acid sequence (SEQ ID NO:72) that comprises the full length 206 amino acids of SNAP25. The forward slash symbol, “/”, marks the cleavage site of BoNT-A protease.

Notably, absent the disclosed platform biosensor system, the longer 61 and 206 amino acid target sequences would have been very difficult to clone into the biosensor gene without the new convertible linker. For example, not only does using non-identical SfiI sites allow directional cloning of a single fragment with virtually no background, as described above, but the rare occurrence of SfiI sites in DNA sequences (estimated 1/65536 bases) makes it highly unlikely that an unwanted SfiI site will occur in the target DNA sequence. The previous restriction enzyme cloning site (BamH1), however, occurs much more frequently (estimated 1/4096 bases), making it more likely that an unwanted BamH1 site will occur in the target DNA sequence. Thus, the convertible linker provides a statistical advantage for cloning any target sequences, which is important because an unwanted SfiI or BamH1 site may render the sequence unclonable.

Those skilled in the art will recognize that other restriction enzyme cleavage sites may be incorporated into the plasmid DNA at desired locations to allow targeted cleavage and insertion of the desired DNA sequences. The efficiency of the platform will be maintained if the overhang sequences and each free and of the cleaved plasmid are different (i.e., non-complementary to each other) to bind to complementary overhang sequences at each free end (also non-complementary to each other) of the DNA segment to be added.

### Example 35

### Assaying BoNT-A Enzyme with Three Different Biosensor Proteins

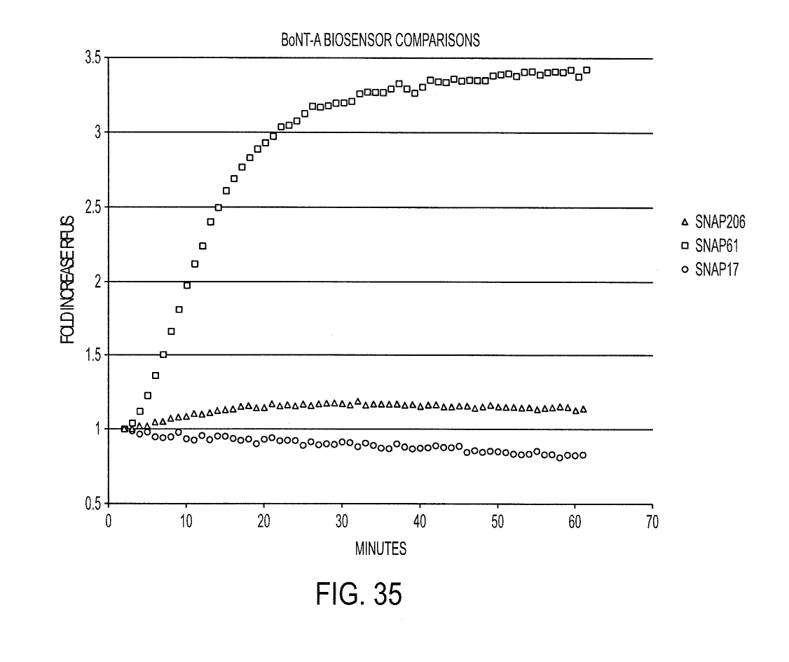

The three different biosensor genes (SNAP17, SNAP61, and SNAP206 of SNAP25 target sequence for BoNT-A protease, each as described above in Example 34, parts a-c) were expressed as biosensor proteins as described in Example 32 with respect to purification of biosensor protein. Each biosensor protein was used to assay the activity of recombinant C. botulinum BoNT A Light Chain (rBoNT/A-LC) (Catalog #4489-ZN, RND Systems). Each assay contained 500 nM biosensor protein, 500 nM MG-2P dye and 5 ngm rBoNT/A-LC, in assay buffer (50 mM HEPES, 0.05% (v/v) TWEEN 20, pH 7.5). Each assay was performed in the well of a 96-well black fluorescence detection plate in a volume of 100 μL. The fluorescence increase at 660 nm following excitation at 635 nm was measured over time. An increase of fluorescence was indicative of successful cleavage of the biosensor by the protease. The results of the assays on each of the three proteins are shown in FIG. 35. The 17 amino acid target biosensor (SNAP17, solid circles) is a poor substrate as it failed to be cleaved by the protease and generate a fluorescent signal. This was expected as these small substrate sequences are relatively poor substrates without chemical modifications (Schmidt, J. J., and Bostian, K. A., Endoproteinase activity of type A botulinum neurotoxin: substrate requirements and activation by serum albumin, J Protein Chem, 16, 19-26 (1997); Schmidt, J. J., and Bostian, K. A., Proteolysis of synthetic peptides by type A botulinum neurotoxin, J Protein Chem, 14, 703-708 (1995)). However, the 61 amino acid target biosensor (SNAP61, solid squares)) proved to be an excellent target sequence, generating a functional BoNT-A protease biosensor as evidenced by the increase of fluorescent signal at least 3-fold above that starting value.

Remarkably, the full-length SNAP25 target containing biosensor (SNAP206) also functions as a BoNT-A biosensor (see solid triangles in FIG. 35). While the rise in fluorescent signal is much less that the SNAP61 biosensor (because the starting fluorescence of the biosensor is significantly higher), it is still activated by BoNT-A. This was unexpected as the V_{H} and V_{L} domains of the biosensor need to be in close proximity for the blocking of dye binding. The length of the inserted target adds 206 amino acids into the middle of the domains, each of which is just over 100 amino acids in length. This probably constrains the protein folding of the biosensor and is likely the reason the starting fluorescence of the biosensor is high.

Those skilled in the art will appreciate upon reading the present disclosure that the disclosed biosensor platform greatly increases efficiency and reduces costs associated with biosensor preparation. For example, the plasmid backbone DNA, when purified and isolated from the small fragment containing the target coding DNA sequence, e.g., the target coding DNA sequence recognized by the HRV-3C protease (see also listed targets in Table 3, below), the plasmid backbone DNA comprises non-complimentary overhang sequences. Therefore, DNA ligase may not re-circularize the plasmid backbone DNA following digestion with SfiI and separation of the insert sequence. In contrast, double-stranded target DNA sequences may be ligated into the plasmid backbone to circularize the plasmid provided they have complementary ends that match those left by SfiI. Beneficially, provided that the two different 3-base overhang sequences on the ends of the target DNA are chosen correctly, the target DNA will join the plasmid in only one direction.

Further to the above, the platform biosensor system has been designed to avoid false positives. For example, the linear plasmid DNA, e.g., plasmid DNA that does not include a successfully inserted target coding DNA sequence, will transform E. coli only very poorly. Therefore, there is no background transformation unless the linear DNA is made circular during insertion of a target sequence. This reduces transformation due to plasmid relegation (background) to zero. Notable, only the ligation of a suitable DNA molecule with the correct ends into the plasmid backbone generates a circular molecule with greatly increased transformation efficiency. Thus, the colonies that appear after ligation and transformation carry plasmids comprising successfully inserted target coding DNA sequences. Because the target DNA has different 3-base, 3′ extensions on each end, the target coding DNA sequence will be ligated into the plasmid in one orientation. Thus, as mentioned above, instead of 50% of the inserts being in the correct orientation (if the target DNA ends are identical), 100% of the inserts will be in the correct orientation. Additionally, because the target DNA has different 3-base, 3′ extensions on each end, one and only one target molecule may be ligated into the plasmid backbone.

As stated above, the new designer biosensor system improves the efficiency and speed of creating new target inserts into the biosensor genes. Using other methods, the insertion of target sequences into the BamH1 restriction site requires isolation of tens to hundreds of different clonal isolates, each one requiring sequencing to identify the correct insert. Those skilled in the art will appreciate that the success rate for such other methods may not be optimal. For example, using such other methods, the success rate for selecting and identifying colonies transformed with successfully incorporated inserts is usually about 1 in 10 to 1 in 25. With the new platform biosensor system, however, even with less than 100 colonies per transformation of a ligation reaction, 2 or 3 colonies may be selected with the expectation of a 100% success rate, e.g., at least 1 positive out of 3 colonies, becomes routine. This has reduced the time required for producing the biosensors by 60% and has significantly reduced the costs, by 88% or more. For example, it will be appreciated by those having skill in the art that there exists a per transformation cost involving the cost of the competent cells as well as a per transformed colony cost that involves the growth of colony into a culture, the cost of preparing plasmid DNA (time as well as kit costs), and DNA sequencing fees.

Using the new platform for the biosensor linker system and the procedures of Examples 30-34 (with substitution of the target DNA sequence in such examples, for DNA sequences that encode for the amino acid sequences of Table 3) disclosed herein, new customized genes containing the sequences that encode for target amino acid sequences described in Table 3 below have been used to successfully create customized biosensors in remarkably less time than heretofore possible. Absent the new platform biosensor linker system, substantially more investment of time and resources would have been required.

TABLE 3

| Target Enzyme | | SEQ ID |
|---|---|---|
| (biosensor name) | Amino acid sequence | NO. |
| HRV-3C Protease | LEVLFQ/GP | 9 |
| recognition sequence | | |
| Caspase 3 Protease | DEVD/ | 77 |
| recognition sequence | | |
| Caspase 1 | YAVD/ | 78 |
| MMP14 | ARG/IKL | 79 |
| MMP2 (2.1) | LQLAL/YTA | 80 |
| MMP2 (2.2) | VQLAY/FTA | 81 |
| MMP2 (2.3) | ATLAA/L | 82 |
| MMP2 (2.4) | FYFSN/L | 83 |
| MMP2 (2.5) | GHPSP/F | 84 |
| MMP2 (2.6) | GYFAN/L | 85 |
| MMP2 (2.7) | PKLAA/I | 86 |
| MMP 2 & 9 | RPSP/FW | 87 |
| Thrombin | TNATLDPR/SFLLRNPNDKYE | 88 |
| | PF | |
| Thrombin | LDPR/SFLLRNP | 89 |
| TEV | ENLYFQ/G | 90 |
| HIV1 protease (MA/CA) | VSQNY/PIVQN | 91 |
| recognition sequence | | |
| HIV1 protease (CA/SP1) | KARVL/AEAMS | 92 |
| HIV1 protease (SP1/NC) | SATIM/MQRGN | 93 |
| HIV1 protease (NC/SP2) | ERQAN/FLGKI | 94 |
| HIV1 protease (SP2/p6gag) | RPGNF/LQSRP | 95 |
| HIV1 protease (NC/TFP) | ERQAN/FLRED | 96 |
| HIV1 protease (TFP/P6pol) | EDLAF/LQGKA | 97 |
| HIV1 protease (P6pol/PR) | VSFNF/PQVTL | 98 |
| HIV1 protease (PR/RT) | CTLNF/PISPI | 99 |
| HIV1 protease | GAETF/YVDGA | 100 |
| (RT/RNaseH) | | |
| HIV1 protease | IRKVL/FLDGI | 101 |
| (RNaseH/IN) | | |
| HIV1 protease (NEF) | AACAW/LEAQE | 102 |
| HIV1 protease (NC/SP2 P2- | ERQVN/FLGKI | 103 |
| A/V) | | |
| HIV1 protease (CA/SP1 P1 | KARVI/AEAMS | 104 |
| L to I) | | |
| HIV1 protease (CA/SP1 P1 | KARVF/AEAMS | 105 |
| L to F) | | |
| HIV1 protease (CA/SP1 P1 | KARVY/AEAMS | 106 |
| L to Y) | | |
| HIV1 protease (HSP90alpha | GDEM/VSLK | 107 |
| 1) | | |
| HIV1 protease | KHIY/YITG | 108 |
| (HSP90alpha | | |
| 2) | | |
| HIV1 protease (MA/CA 20) | GHSNQVSQNY/PIVQNIQGQ | 109 |
| | M | |
| HIV1 protease (MA/CA 30) | AAADTGHSNQVSQNY/PIVQ | 110 |
| | NIQGQMVHQAI | |
| HIV1 protease (CA/sp1 25) | GVGGPGHKARVL/AEAMSQ | 111 |
| | VTNSATI | |
| HCV-NS4A/4B | DEMEEC/ASHL | 112 |
| Thrombin (A1) | TNATLDPR/SFLLRNPNDKYE | 113 |
| | PF | |
| ADAM10 | PRYEA/YKMG | 114 |
| ADAM10 | PRAEA/LKGG | 115 |
| TACE | PRAAA/VKSP | 116 |
| TNF | PLAQA/VRSS | 117 |

In certain embodiments, the biosensor may be immobilized onto a substrate surface, including, for example, substrates such as silicon, silica, quartz, glass, controlled pore glass, carbon, alumina, titania, tantalum oxide, germanium, silicon nitride, zeolites, gallium arsenide, gold, platinum, aluminum, copper, titanium, alloys, polystyrene, poly(tetra)fluoroethylene (PTFE), polyvinylidenedifluoride, polycarbonate, polymethylmethacrylate, polyvinylethylene, polyethyleneimine, poly(etherether)ketone, polyoxymethylene (POM), polyvinylphenol, polylactides, polymethacrylimide (PMI), polyalkenesulfone (PAS), polypropylethylene, polyethylene, polyhydroxyethylmethacrylate (HEMA), polydimethylsiloxane, polyacrylamide, polyimide, and block-copolymers. Such substrates may be in the form of beads, chips, plates, slides, strips, sheets, films, blocks, plugs, medical devices, surgical instruments, diagnostic instruments, drug delivery devices, prosthetic implants, and other structures.

One or more biosensors may be bound to discrete beads or microspheres. The microspheres typically are either carboxylated or avidin-modified so that proteins, such as antibodies, non-antibody receptors and variants and fragments thereof, may be readily attached to the beads by standard chemistries. In an exemplary embodiment, the scFv portions of the biosensors may be bound to carboxylated beads by one of many linking chemistries, such as, for example, EDC chemistry, or bound to avidin-coated beads by first biotinylating the scFv fragment by one of many common biotinylation chemistries, such as, for example, by conjugation with sulfo-NHS-LC-biotin). In one embodiment, the biosensors are affixed to a substrate in a tiled array, with each biosensor represented in one or more positions in the tiled array. The spatial configuration of the substrate or substrates may be varied so long as each biosensor species is bound at detectably discrete locations.

The biosensors produced by any of the foregoing procedures may be used in various applications, such as in assays to detect protease activity. Several representative assays are described herein. The following assays may be used, for example, for diagnostic purposes to determine the presence and level of target enzymes.

### Example 36

### Fluorescence Based Activity Assay for Micro-Well Plate

Buffer, 200 nM biosensor of the present invention (i.e., a protease detection substrate), and 500 nM fluorogenic dye selected from any of the dyes described herein were added in a plate well or a microfuge tube. The buffer used consisted of 2× Tris buffered saline (TBS)+0.2% Pluronic F-127 (Invitrogen Cat# P6867), 16 g NaCl, 0.4 g KCl, 6 g Tris base, in 1 L, adjusted to pH 7.4 with HCl. The dye:biosensor substrate in buffer mixture was vortexed for 3 seconds on medium speed. The final dye concentration was 100 nM. The biosensor substrate:dye mixture was incubated at room temperature, between 4° C. and 37° C., for 5 minutes protected from light. A 50 μL test sample was added and vortexed for 3 seconds on medium speed. Then, the plate or tube was read at 620/660 nm for endpoint and kinetic curve assays. For fluorescence detection, the excitation wavelength is from 625-635 nm and the emission wavelength is from 655-665 nm. This general protocol may be used for fluorimetric based assays.

Detection can be performed using any fluorescence detection equipment where a beam of light excites the electrons and causes them to emit light detected by the equipment. Exemplary fluorescent detection means include fluorescence microscopes, cuvette or micro-well plate filter fluorimeters or spectrofluorimeter, flow cytometer including fluorescence-activated cell sorting (FACS), and high performance liquid chromatography (HPLC).

### Example 37

### Fluorimetric Based Assays for Tissue Samples

A protocol for an assay of tissue on slides using the FAPs as stain proceeds as follows: A 5 micron thick section of abdominal aortic aneurysm tissue was unfixed, flash frozen and mounted onto a slide in a cryostat. Multiple biosensor reagent concentrations in doubling dilutions were used to test for optimal signal. The biosensor reagent was diluted for testing at 1:10, 1:20, 1:40, etc. up to 1:1280 serial dilutions. The optimal concentration is believed to be in the range of 1:160 plus or minus one dilution. The dilutions correspond to the following biosensor molar concentrations: 1:80=347 nM, 1:160=173 nM, and 1:320=87 nM.

The sample was treated with biosensor reagent as follows: To the slides with the tissue fixed thereto, MMP 25 biosensor was added. The tissue slides were incubated at 37° C. with MMP 25 biosensor for 1 hour. A fluorogenic dye was added to 50 nM final concentration and incubate for 30 minutes. The labeled tissue was mounted in Gelvatol™ and cover slipped with no further washes.

Gelvatol is prepared from PVA—Sigma Chemical Cat. #P-8136, Glycerol—Sigma Chemical Cat. #G-9012, and Sodium Azide—Fisher Chemical Cat. #S227-100, as follows: Add 21 g PVA to 42 mL glycerol until the solution is clear and is slightly less viscous than molasses. Then add 52 mL dH₂0, add a few crystals of sodium azide, add 106 mL Tris (0.2M, pH=8.5), and stir with low heat for a few hours until reagents dissolve completely. Refrigerate the gelvatol overnight at 4° C. to ensure that the viscosity is that of molasses. If the mixture is too viscous, add more glycerol to bring the viscosity down. If the mixture is not viscous enough, add more PVA with heat and then refrigerate for about 1-3 hours or more as needed until the viscosity is that of molasses. Then, clarify the mixture by centrifugation at 5000 g for 15 minutes and aliquot and store at 4° C.

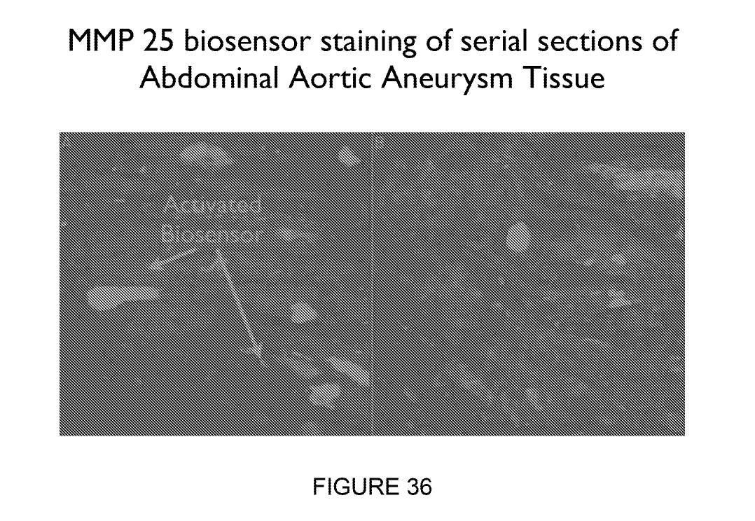

The MMP 25 stained tissue is shown in FIG. 36. Panel A shows the biosensor treated tissue section. Human abdominal aortic aneurysm tissue has many different proteases in it. The left hand panel, A, shows the results of tissue stained with the MMP 25 embodiment of the biosensor described herein. The arrows point to bright or dull fluorescence (red) from an activated MMP 25 biosensor, indicating the presence of the protease, MMP 25 in the tissue. The fluorescent color in Panel A (not visible in the grey scale figures) is the signal from the activated biosensor. The signal is diffuse throughout the collagen bed with small (cell-sized) brighter spots. There is extensive autofluorescence of the tissue. Additional brightness (bright orange/yellow, not visible in grey scale figures) is attributable to paracystalline plaque, which is ubiquitous. The right hand panel, B, shows a control experiment that was treated with a biosensor that was not specific for MMP25 sensor. There is no similar coloration in panel B.

The biosensors described herein may be used to detect proteases in various types of tissue samples, such as, for example, secreted proteases in biopsy tissue, circulating proteases in bodily fluids, such as blood, serum, spinal fluid, urine, etc., and tissue aspirates. Any one or more such biosensors may be used as appropriate for the analyte and/or activity being assayed.

Embodiments within the scope of the invention described herein may include the following.

A biosensor comprising a fluorogen-activating peptide having an active domain; and a blocking peptide linked to the fluorogen-activating peptide, wherein one of the fluorogen-activating peptide and the blocking peptide comprises a variable heavy chain domain of an antibody and the other peptide comprises a variable light chain domain of a different antibody.

A biosensor comprising a fluorogen-activating peptide having an active domain; and a blocking peptide linked to the fluorogen-activating peptide through a peptide linker comprising an amino acid sequence that is specifically recognized as a modification substrate by a cognate enzyme, and in various embodiments, as a cleavage substrate by a cognate enzyme, wherein the blocking peptide associates with the fluorogen-activating peptide thereby blocking an active domain of the fluorogen-activating peptide when the peptide linker is unmodified, or uncleaved, and wherein the fluorogen-activating peptide and the blocking peptide at least partially disassociate when the linker is modified or cleaved by a cognate enzyme, thereby allowing the fluorogen-activating peptide to interact with a cognate fluorogen and modulate a fluorescence signal. In this embodiment of a biosensor, the fluorogen-activating peptide may comprise a variable heavy chain domain of an antibody and the blocking peptide may comprise a variable light chain domain of a different antibody. Alternatively, the fluorogen-activating peptide may comprise a variable light chain domain of an antibody and the blocking peptide may comprise a variable heavy chain domain of a different antibody.

In any of the embodiments of biosensors described herein, the fluorogen-activating peptide may specifically bind to a cognate fluorogen. The cognate fluorogen may be selected from the group consisting of thiazole orange, malachite green, dimethyl indole red, and derivatives thereof.

In various embodiments of the biosensors described herein, the peptide linker may comprise an amino acid sequence that is specifically recognized as a cleavage substrate by a cognate protease, wherein the fluorogen-activating peptide and the blocking peptide at least partially disassociate when the linker is cleaved by a cognate protease, thereby allowing the fluorogen-activating peptide to interact with a cognate fluorogen and modulate a fluorescence signal. The peptide linker may comprise an amino acid sequence specifically recognized as a cleavage substrate by a protease. The linker may comprise an amino acid sequence specifically recognized as a cleavage substrate by furin, and may for example, comprise an amino acid sequence comprising the sequence Arg-Xaa-(Arg/Lys)-Arg, such as Arg-Lys-Lys-Arg-Ser (SEQ ID NO: 3), or Asn-Ser-Arg-Lys-Lys-Arg-Ser-Thr-Ser-Ala (SEQ. ID NO: 5). The linker may comprise an amino acid sequence specifically recognized as a cleavage substrate by matrix metalloproteinase 25, and may for example, comprise an amino acid sequence comprising the sequence Val-Met-Arg-Leu-Val-Val (SEQ. ID NO: 15). The linker may comprise an amino acid sequence specifically recognized as a cleavage substrate by human rhinovirus protease 3C, and may for example comprise an amino acid sequence comprising the sequence Leu-Glu-Val-Leu-Phe-Gln-Gly-Pro (SEQ. ID NO: 9). The linker may comprise an amino acid sequence specifically recognized as a cleavage substrate by a caspase, and may for example comprise an amino acid sequence specifically recognized as a cleavage substrate by caspase 1, such as Tyr-Ala-Val-Asp (SEQ. ID NO: 11). The linker may comprise an amino acid sequence specifically recognized as a cleavage substrate by caspase 3, and may for example comprise an amino acid sequence comprising the sequence Asp-Glu-Val-Asp (SEQ. ID NO: 13). The linker may comprise an amino acid sequence specifically recognized as a cleavage substrate by TEV protease, and may for example, comprises an amino acid sequence comprising the sequence Glu-Asn-Leu-Tyr-Phe-Gln-Gly (SEQ. ID NO: 7). The peptide linker may comprise an amino acid sequence that is specifically recognized as a phosphorylation substrate by a cognate protein kinase. For example, such a linker may comprise an amino acid sequence specifically recognized by protein kinase A as a phosphorylatable peptide sequence, such as the sequence Leu-Leu-Arg-Arg-Ala-Ser-Leu-Gly-Pro (SEQ. ID NO: 17). For example, such a linker may comprise a phospho(amino acid) binding peptide linked to the fluorogen-activating peptide or the blocking peptide, such as the 14-3-3τ domain. The linker may comprise an amino acid sequence that is specifically recognized as an acetylation substrate by a cognate acetyltransferase, and may for example, comprise an amino acid sequence comprising a Lys residue, wherein the amino acid sequence is specifically recognized by a histone acetyltransferase as an acetylatable peptide sequence, such the sequence Ile-Arg-Arg-Phe-Gln-Lys-Ser-Thr-Asp-Leu-Leu (SEQ. ID NO: 19). The linker may comprise a bromo-domain, wherein the bromo-domain peptide is linked to the fluorogen-activating peptide or the blocking peptide. The linker may comprise an amino acid sequence having at least 85%-100%, at least 90%-100%, at least 95%-100%, or at least 98%-100%, or complete sequence identity to the amino acid sequences of SEQ. ID NO. 71 and 77-117. Those skilled in the art will recognize that depending on the plasmid used and the restriction enzymes used, and other well understood variables of the standard cloning and expression techniques, alterations in the number and position of certain residues in the linker sequence that are not critical to the function of the biosensor's cleavable peptide linker will vary and that such variation is contemplated and does not detract from the unique aspects of the blocking biosensor construct and its unique convertible linker segment.

An embodiment of a biosensor may comprising a fluorogen-activating peptide comprising a variable domain of an antibody, and a blocking peptide comprising a variable domain of an antibody, wherein one of the fluorogen-activating peptide and the blocking peptide comprises a variable heavy chain domain of an antibody and the other peptide comprises a variable light chain domain of a different antibody, and wherein the blocking peptide is linked to the fluorogen-activating peptide through a peptide linker comprising an amino acid sequence that is specifically recognized as a cleavage substrate by a cognate protease, and wherein the blocking peptide associates with the fluorogen-activating peptide thereby blocking an active domain of the fluorogen-activating peptide when the linker is intact, and wherein the fluorogen-activating peptide and the blocking peptide disassociate when the linker is cleaved by a cognate protease, thereby allowing the fluorogen-activating peptide to interact with a cognate fluorogen and modulate a fluorescence signal.

Embodiments described herein include a composition comprising any of the biosensors described herein and a fluorogen. The fluorogen may be selected from the group consisting of thiazole orange, malachite green, dimethyl indole red, and derivatives thereof.

Embodiments described herein include a method for analyzing enzyme activity comprising contacting a medium comprising an analyte enzyme with the composition described herein, and detecting a fluorescence signal produced by an interaction between the fluorogen-activating peptide and the fluorogen.

Embodiments described herein include a method for analyzing enzyme activity comprising contacting a reaction medium comprising an analyte enzyme with a composition comprising a fluorogen and a biosensor that comprises a fluorogen-activating peptide and a blocking peptide linked to the fluorogen-activating peptide through a peptide linker, and detecting a fluorescence signal produced by an interaction between the fluorogen-activating peptide and the fluorogen. The peptide linker may comprise an amino acid sequence that is specifically recognized as a modification substrate by a cognate enzyme, wherein the blocking peptide associates with the fluorogen-activating peptide thereby blocking an active domain of the fluorogen-activating peptide when the peptide linker is unmodified, and wherein the fluorogen-activating peptide and the blocking peptide at least partially disassociate when the linker is modified by a cognate enzyme, thereby allowing the fluorogen-activating peptide to bind a cognate fluorogen and modulate a fluorescence signal.

The constructs and methods described herein may serve as a platform for the development and construction of numerous other specific biosensors not expressly disclosed herein. The constructs and methods described herein are extendable to other analytes and other fluorogen-activating peptides, blocking peptides, and peptide linkers. In this manner, numerous biosensors comprising various different fluorogen-activating peptides, blocking peptides and peptide linkers may be developed and constructed.

All patents, patent applications, publications, or other disclosure material mentioned herein, are hereby incorporated by reference in their entirety as if each individual reference was expressly incorporated by reference respectively. All references said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference. Any material, or portion thereof, that is said to be incorporated by reference herein, but which conflicts with existing definitions, statements, or other disclosure material set forth herein is only incorporated to the extent that no conflict arises between that incorporated material and the existing disclosure material. In case of conflict, the disclosure expressly set forth in the present application controls.

The present invention has been described with reference to various exemplary and illustrative embodiments. The embodiments described herein are understood as providing illustrative features of varying detail of various embodiments of the disclosed invention; and therefore, unless otherwise specified, the features, elements, components, constituents, ingredients, structures, modules, and/or aspects of the disclosed embodiments may be combined, separated, interchanged, and/or rearranged without departing from the scope of the disclosed invention. Accordingly, it will be recognized by persons having ordinary skill in the art that various substitutions, modifications or combinations of any of the exemplary embodiments may be made without departing from the scope of the invention. In addition, persons skilled in the art will recognize, or be able to ascertain using no more than routine experimentation, many equivalents to the various embodiments of the invention described herein upon review of this specification. Thus, the invention is not limited by the description of the various embodiments, but rather by the claims.

1. A recombinant DNA molecule encoding a biosensor, the recombinant DNA molecule comprising:
a first DNA sequence encoding a fluorogen-activating peptide;
a second DNA sequence encoding a blocking peptide;
wherein one of the fluorogen-activating peptide and the blocking peptide comprises a variable heavy chain domain of an antibody and the other peptide comprises a variable light chain domain of a different antibody; and
a convertible linker DNA sequence positioned between the first DNA sequence and the second DNA sequence, wherein the convertible linker DNA sequence comprises:
a first restriction enzyme cleavage site;
a second restriction enzyme cleavage site; and
a first target DNA sequence positioned between the first restriction enzyme cleavage site and the second restriction enzyme cleavage site such that the first target DNA sequence is excised upon digestion, in use, with at least one restriction enzyme that cleaves the first cleavage site and the second cleavage site;
wherein the DNA molecule has, upon digestion, in use, with the first restriction enzyme two different, non-complementary overhang sequences.

2. The recombinant DNA molecule of claim 1, wherein the first cleavages site and the second cleavage site are cleavable by a first restriction enzyme.

3. (canceled)

4. The recombinant DNA molecule of claim 1, wherein the DNA molecule is circular and is linearized upon excision of the target sequence by digestion, in use, with a restriction enzyme that cleaves the first cleavage site and the second cleavage site.

5. The recombinant DNA molecule of claim 4, wherein the linearized recombinant DNA molecule will not recircularize absent ligation to a second target DNA sequence comprising non-complementary overhang sequences that are complimentary to the non-complementary overhang sequences of the recombinant DNA molecule.

6. The recombinant DNA molecule of claim 1, further comprising a second target DNA sequence having 5′ and 3′ ends, each complementary to one of the non-complementary overhang sequences of the recombinant DNA molecule in only one orientation.

7. The recombinant DNA molecule of claim 6, wherein the second target DNA sequence comprises a first overhang sequence and a second overhang sequence, wherein the first overhang sequence of the second target DNA sequence is complementary to one of the overhang sequences of the linearized recombinant DNA molecule and the second overhang sequence of the second target DNA sequence is complementary to the other overhang sequence of the linearized recombinant DNA molecule.

8. A method comprising:
excising a target DNA sequence by digesting a plasmid comprising the recombinant DNA molecule of claim 1 with a restriction enzyme that cleaves the first cleavage site and the second cleavage site.

9. The method of claim 8, wherein the step of excising the target DNA sequence linearizes the recombinant DNA molecule to produce a first overhang sequence and a second overhang sequence on free ends of the linearized DNA molecule, wherein the first overhang sequence and the second overhang sequence are non-complementary to each other.

10. The method of claim 9, further comprises ligating a first end of a second DNA target sequence to the first overhang sequence of the linearized recombinant DNA molecule and ligating a second end of the second DNA target sequence to the second overhang sequence of the linearized recombinant DNA molecule, wherein the first end of the second DNA target sequence comprises an overhang sequence complementary to the first overhang sequence of the linearized recombinant DNA molecule and the second end of the second DNA target sequence comprises an overhang sequence complementary to the second overhang sequence of the linearized recombinant DNA molecule.

11. The method of claim 10, wherein ligating the second DNA target sequence circularizes the recombinant DNA sequence.

12. The method of claim 10 further comprising:
transforming a host cell with the recombinant DNA molecule encoding the biosensor;
culturing the transformed cell under suitable conditions to allow expression of a recombinant polypeptide comprising the biosensor; and
recovering the recombinant polypeptide comprising the biosensor from the cell culture.

13. The method of claim 8, wherein the convertible linker DNA sequence of claim 1 comprises a control DNA sequence, wherein the control DNA sequence does not overlap with the target DNA sequence, and wherein the control DNA sequence encodes an amino acid sequence recognized as a cleavage substrate of a cognate enzyme.

14. The method of claim 8 wherein the target DNA sequence encodes a peptide linker comprised of an amino acid sequence that is specifically recognized as a cleavage substrate by a cognate enzyme.

15. The method of claim 8 wherein the convertible linker DNA sequence of the recombinant DNA molecule of claim 1 comprises a first SfiI recognition sequence, a second SfiI recognition sequence that differs from the first SfiI recognition sequence, and a target DNA sequence positioned therebetween that encodes a peptide linker comprised of an amino acid sequence that is specifically recognized as a cleavage substrate by a cognate enzyme.

16. The method of claim 8 wherein the convertible linker DNA sequence of the recombinant DNA molecule of claim 1 comprises a restriction enzyme cleavage site having at least one variation of the nucleotide sequence of SEQ. ID. NO. 74.

17. The method of claim 16 wherein the convertible linker DNA sequence of the recombinant DNA molecule of claim 1 comprises two restriction enzyme cleavage sites each having a different variation of the nucleotide sequence of SEQ. ID. NO. 74.

18. A composition comprising:
a biosensor encoded by the recombinant DNA molecule of claim 1; and
a fluorogen.

19. A method for analyzing enzyme activity comprising:
contacting a reaction medium suspected of containing at least one enzyme of interest with at least one composition comprising a biosensor and a cognate fluorogen, the biosensor being encoded by the recombinant DNA molecule of claim 1, and comprising a fluorogen-activating peptide having an active domain and a blocking peptide linked to the fluorogen-activating peptide through a peptide linker comprising an amino acid sequence that is specifically recognized as a cleavage substrate by the at least one enzyme, wherein one of the fluorogen-activating peptide and the blocking peptide comprises a variable heavy chain domain of an antibody and the other peptide comprises a variable light chain domain of a different antibody; and
detecting for a fluorescence signal produced by an interaction between at least one fluorogen-activating peptide and the cognate fluorogen thereof to determine the presence of at least one said enzyme.

20. The method of claim 19 wherein the enzyme is a protease.

21. The method of claim 19 wherein the fluorogen is selected from the group consisting of thiazole orange, malachite green, dimethyl indole red, and derivatives thereof.

22. A vector comprising a nucleic acid sequence of claim 1.

23. A host cell comprising the vector of claim 22.

24. An isolated, purified biosensor encoded by the recombinant DNA molecule of claim 1.

25. A host cell expressing the biosensor of claim 24.
